(19) United States
(12) Patent Application Publication
(10) Pub. No.: US 2023/0272082 A1
(43) Pub. Date: Aug. 31, 2023

(54) ANTI-VISTA ANTIBODIES AND USES THEREOF

(71) Applicant: Sensei Biotherapeutics, Inc., Rockville, MD (US)

(72) Inventors: Robert PIERCE, Seattle, WA (US); Edward H. VAN DER HORST, Palo Alto, CA (US); Thomas THISTED, New Market, MD (US); Nadthakarn BOLAND, Lebanon, NH (US); Nels NIELSON, Lebanon, NH (US)

(73) Assignee: Sensei Biotherapeutics, Inc., Rockville, MD (US)

(21) Appl. No.: 18/053,996

(22) Filed: Nov. 9, 2022

Related U.S. Application Data
(60) Provisional application No. 63/376,554, filed on Sep. 21, 2022.
(60) Provisional application No. 63/374,147, filed on Aug. 31, 2022.
(60) Provisional application No. 63/332,813, filed on Apr. 20, 2022.
(60) Provisional application No. 63/277,395, filed on Nov. 9, 2021.

(51) Int. Cl. C07K 16/28; A61P 35/00; A61P 1/00

(52) U.S. Cl. CPC C07K 16/2827; C07K 16/2818; A61P 35/00; A61P 1/00; C07K 2317/21; A61K 2039/505

(57) ABSTRACT

Provided herein are antibody molecules that bind specifically to VISTA and antigen-binding portions thereof and related compositions, nucleic acid molecules, vectors and host cells. Also provided herein are medical uses of such antibody molecules.

### CROSS REFERENCE TO RELATED APPLICATIONS

This application claims the benefit of U.S. Provisional Patent Application No. 63/277,395, filed Nov. 9, 2021, U.S. Provisional Patent Application No. 63/332,813, filed Apr. 20, 2022, U.S. Provisional Patent Application No. 63/374,147, filed Aug. 31, 2022, and U.S. Provisional Patent Application No. 63/376,554, filed Sep. 21, 2022, the disclosure of each of which is hereby incorporated by reference in its entirety.

### REFERENCE TO AN ELECTRONIC SEQUENCE LISTING

The contents of the electronic sequence listing (SEBI_024_001 US_SeqList_ST26.xml; Size: 152,546 bytes; and Date of Creation: Nov. 7, 2022) are herein incorporated by reference in their entirety.

### TECHNICAL FIELD

The present disclosure relates to therapeutic antibody molecules and medical uses thereof.

### BACKGROUND

V-domain immunoglobulin suppressor of T-cell activation (VISTA) is a B7-family member type I transmembrane protein that functions in immune regulation. VISTA is primarily expressed in hematopoietic cells and promotes T-cell and myeloid quiescence. VISTA expression correlates with poor survival rates across multiple cancer indications. The interaction of VISTA with its receptor PSGL-1 was demonstrated to be significantly enhanced by the acidic tumor microenvironment (TME) (Johnston et al. Nature 574, 565-570 (2019)). There is a need for therapeutics that target VISTA.

### SUMMARY

Provided herein is an anti-V-domain immunoglobulin suppressor of T-cell activation (VISTA) antibody or an antigen-binding portion thereof, wherein the antibody or antigen-binding portion comprises a heavy chain variable (VH) region and a light chain variable (VL) region wherein: (a) the VH region amino acid sequence comprises a HCDR1 comprising SEQ ID NO: 54, a HCDR2 comprising SEQ ID NO: 55 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (b) the VH region amino acid sequence comprises a HCDR1 comprising SEQ ID NO: 60, a HCDR2 comprising SEQ ID NO: 61 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (c) the VH region amino acid sequence comprises a HCDR1 comprising SEQ ID NO: 62, a HCDR2 comprising SEQ ID NO: 63 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (d) the VH region amino acid sequence comprises a HCDR1 comprising SEQ ID NO: 64, a HCDR2 comprising SEQ ID NO: 65 and a HCDR3 comprising SEQ ID NO: 66; and the VL region amino acid sequence comprises a LCDR1 comprising SEQ ID NO: 57, a LCDR2 comprising SEQ ID NO: 67 and a LCDR3 comprising SEQ ID NO: 68; (e) the VH region amino acid sequence comprises a HCDR1 comprising SEQ ID NO: 69, a HCDR2 comprising SEQ ID NO: 70 and a HCDR3 comprising SEQ ID NO: 56; and the VL region amino acid sequence comprises a LCDR1 comprising SEQ ID NO: 57, a LCDR2 comprising SEQ ID NO: 67 and a LCDR3 comprising SEQ ID NO: 71; (f) the VH region amino acid sequence comprises a HCDR1 comprising SEQ ID NO: 60, a HCDR2 comprising SEQ ID NO: 61 and a HCDR3 comprising SEQ ID NO: 72; and the VL region amino acid sequence comprises a LCDR1 comprising SEQ ID NO: 57, a LCDR2 comprising SEQ ID NO: 58 and a LCDR3 comprising SEQ ID NO: 68; (g) the VH region amino acid sequence comprises a HCDR1 comprising SEQ ID NO: 73, a HCDR2 comprising SEQ ID NO: 65 and a HCDR3 comprising SEQ ID NO: 74; and the VL region amino acid sequence comprises a LCDR1 comprising SEQ ID NO: 57, a LCDR2 comprising SEQ ID NO: 58 and a LCDR3 comprising SEQ ID NO: 71; (h) the VH region amino acid sequence comprises a HCDR1 comprising SEQ ID NO: 64, a HCDR2 comprising SEQ ID NO: 75 and a HCDR3 comprising SEQ ID NO: 66; and the VL region amino acid sequence comprises a LCDR1 comprising SEQ ID NO: 57, a LCDR2 comprising SEQ ID NO: 76 and a LCDR3 comprising SEQ ID NO: 68; (i) the VH region amino acid sequence comprises a HCDR1 comprising SEQ ID NO: 64, a HCDR2 comprising SEQ ID NO: 65 and a HCDR3 comprising SEQ ID NO: 77; and the VL region amino acid sequence comprises a LCDR1 comprising SEQ ID NO: 57, a LCDR2 comprising SEQ ID NO: 78 and a LCDR3 comprising SEQ ID NO: 68; (j) the VH region amino acid sequence comprises a HCDR1 comprising SEQ ID NO: 60, a HCDR2 comprising SEQ ID NO: 75 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 68; (k) the VH region amino acid sequence comprises a HCDR1 comprising SEQ ID NO: 54, a HCDR2 comprising SEQ ID NO: 65 and a HCDR3 comprising SEQ ID NO: 79; and the VL region amino acid sequence comprises a LCDR1 comprising SEQ ID NO: 80, a LCDR2 comprising SEQ ID NO: 58 and a LCDR3 comprising SEQ ID NO: 68; (1) the VH region amino acid sequence comprises a HCDR1 comprising SEQ ID NO: 81, a HCDR2 comprising SEQ ID NO: 82 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (m) the VH region amino acid sequence comprises a HCDR1 comprising SEQ ID NO: 87, a HCDR2 comprising SEQ ID NO: 88 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (n) the VH region amino acid sequence comprises a HCDR1 comprising SEQ ID NO: 89, a HCDR2 comprising SEQ ID NO: 90 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (o) the VH region amino acid sequence comprises a HCDR1 comprising SEQ ID NO: 91, a HCDR2 comprising SEQ ID NO: 92 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (p) the VH region amino acid sequence comprises a HCDR1 comprising SEQ ID NO: 93, a HCDR2 comprising SEQ ID NO: 94 and a HCDR3 comprising SEQ ID NO: 95; and the VL region amino acid sequence comprises a LCDR1 comprising SEQ ID NO: 96, a LCDR2 comprising SEQ ID NO: 85 and a LCDR3 comprising SEQ ID NO: 97; (q) the VH region amino acid sequence comprises a HCDR1 comprising SEQ ID NO: 98, a HCDR2 comprising SEQ ID NO: 99 and a HCDR3 comprising SEQ ID NO: 100; and the VL region amino acid sequence comprises a LCDR1 comprising SEQ ID NO: 96, a LCDR2 comprising SEQ ID NO: 101 and a LCDR3 comprising SEQ ID NO: 102; (r) the VH region amino acid sequence comprises a HCDR1 comprising SEQ ID NO: 103, a HCDR2 comprising SEQ ID NO: 104 and a HCDR3 comprising SEQ ID NO: 105; and the VL region amino acid sequence comprises a LCDR1 comprising SEQ ID NO: 96, a LCDR2 comprising SEQ ID NO: 106 and a LCDR3 comprising SEQ ID NO: 107; (s) the VH region amino acid sequence comprises a HCDR1 comprising SEQ ID NO: 108, a HCDR2 comprising SEQ ID NO: 94 and a HCDR3 comprising SEQ ID NO: 105; and the VL region amino acid sequence comprises a LCDR1 comprising SEQ ID NO: 109, a LCDR2 comprising SEQ ID NO: 85 and a LCDR3 comprising SEQ ID NO: 110; (t) the VH region amino acid sequence comprises a HCDR1 comprising SEQ ID NO: 111, a HCDR2 comprising SEQ ID NO: 94 and a HCDR3 comprising SEQ ID NO: 112; and the VL region amino acid sequence comprises a LCDR1 comprising SEQ ID NO: 96, a LCDR2 comprising SEQ ID NO: 85 and a LCDR3 comprising SEQ ID NO: 113; (u) the VH region amino acid sequence comprises a HCDR1 comprising SEQ ID NO: 87, a HCDR2 comprising SEQ ID NO: 94 and a HCDR3 comprising SEQ ID NO: 114; and the VL region amino acid sequence comprises a LCDR1 comprising SEQ ID NO: 115, a LCDR2 comprising SEQ ID NO: 85 and a LCDR3 comprising SEQ ID NO: 116; (v) the VH region amino acid sequence comprises a HCDR1 comprising SEQ ID NO: 93, a HCDR2 comprising SEQ ID NO: 90 and a HCDR3 comprising SEQ ID NO: 117; and the VL region amino acid sequence comprises a LCDR1 comprising SEQ ID NO: 118, a LCDR2 comprising SEQ ID NO: 85 and a LCDR3 comprising SEQ ID NO: 86; (w) the VH region amino acid sequence comprises a HCDR1 comprising SEQ ID NO: 119, a HCDR2 comprising SEQ ID NO: 120 and a HCDR3 comprising SEQ ID NO: 121; and the VL region amino acid sequence comprises a LCDR1 comprising SEQ ID NO: 57, a LCDR2 comprising SEQ ID NO: 58 and a LCDR3 comprising SEQ ID NO: 122; (x) the VH region amino acid sequence comprises a HCDR1 comprising SEQ ID NO: 123, a HCDR2 comprising SEQ ID NO: 124 and a HCDR3 comprising SEQ ID NO: 125; and the VL region amino acid sequence comprises a LCDR1 comprising SEQ ID NO: 126, a LCDR2 comprising SEQ ID NO: 127 and a LCDR3 comprising SEQ ID NO: 128; (y) the VH region amino acid sequence comprises a HCDR1 comprising SEQ ID NO: 123, a HCDR2 comprising SEQ ID NO: 124 and a HCDR3 comprising SEQ ID NO: 129; and the VL region amino acid sequence comprises a LCDR1 comprising SEQ ID NO: 57, a LCDR2 comprising SEQ ID NO: 58 and a LCDR3 comprising SEQ ID NO: 130; (z) the VH region amino acid sequence comprises a HCDR1 comprising SEQ ID NO: 131, a HCDR2 comprising SEQ ID NO: 132 and a HCDR3 comprising SEQ ID NO: 133; and the VL region amino acid sequence comprises a LCDR1 comprising SEQ ID NO: 126, a LCDR2 comprising SEQ ID NO: 134 and a LCDR3 comprising SEQ ID NO: 135; (aa) the VH region amino acid sequence comprises a HCDR1 comprising SEQ ID NO: 131, a HCDR2 comprising SEQ ID NO: 132 and a HCDR3 comprising SEQ ID NO: 136; and the VL region amino acid sequence comprises a LCDR1 comprising SEQ ID NO: 57, a LCDR2 comprising SEQ ID NO: 58 and a LCDR3 comprising SEQ ID NO: 137; (bb) the VH region amino acid sequence comprises a HCDR1 comprising SEQ ID NO: 54, a HCDR2 comprising SEQ ID NO: 138 and a HCDR3 comprising SEQ ID NO: 139; and the VL region amino acid sequence comprises a LCDR1 comprising SEQ ID NO: 57, a LCDR2 comprising SEQ ID NO: 58 and a LCDR3 comprising SEQ ID NO: 140; or (cc) the VH region amino acid sequence comprises a HCDR1 comprising SEQ ID NO: 81, a HCDR2 comprising SEQ ID NO: 82 and a HCDR3 comprising SEQ ID NO: 141; and the VL region amino acid sequence comprises a LCDR1 comprising SEQ ID NO: 142, a LCDR2 comprising SEQ ID NO: 143 and a LCDR3 comprising SEQ ID NO: 144.

Provided herein is an anti-VISTA antibody or an antigen-binding portion thereof, wherein the antibody or antigen-binding portion comprises a VH region and a VL region wherein: (a) the VH region amino acid sequence comprises SEQ ID NO: 1 and the VL region amino acid sequence comprises SEQ ID NO: 2; (b) the VH region amino acid sequence comprises SEQ ID NO: 3 and the VL region amino acid sequence comprises SEQ ID NO: 2; (c) the VH region amino acid sequence comprises SEQ ID NO: 4 and the VL region amino acid sequence comprises SEQ ID NO: 2; (d) the VH region amino acid sequence comprises SEQ ID NO: 5 and the VL region amino acid sequence comprises SEQ ID NO: 6; (e) the VH region amino acid sequence comprises SEQ ID NO: 7 and the VL region amino acid sequence comprises SEQ ID NO: 8; (f) the VH region amino acid sequence comprises SEQ ID NO: 9 and the VL region amino acid sequence comprises SEQ ID NO: 10; (g) the VH region amino acid sequence comprises SEQ ID NO: 11 and the VL region amino acid sequence comprises SEQ ID NO: 12; (h) the VH region amino acid sequence comprises SEQ ID NO: 13 and the VL region amino acid sequence comprises SEQ ID NO: 14; (i) the VH region amino acid sequence comprises SEQ ID NO: 15 and the VL region amino acid sequence comprises SEQ ID NO: 16; (j) the VH region amino acid sequence comprises SEQ ID NO: 17 and the VL region amino acid sequence comprises SEQ ID NO: 18; (k) the VH region amino acid sequence comprises SEQ ID NO: 19 and the VL region amino acid sequence comprises SEQ ID NO: 20; (1) the VH region amino acid sequence comprises SEQ ID NO: 21 and the VL region amino acid sequence comprises SEQ ID NO: 22; (m) the VH region amino acid sequence comprises SEQ ID NO: 23 and the VL region amino acid sequence comprises SEQ ID NO: 22; (n) the VH region amino acid sequence comprises SEQ ID NO: 24 and the VL region amino acid sequence comprises SEQ ID NO: 22; (o) the VH region amino acid sequence comprises SEQ ID NO: 25 and the VL region amino acid sequence comprises SEQ ID NO: 22; (p) the VH region amino acid sequence comprises SEQ ID NO: 26 and the VL region amino acid sequence comprises SEQ ID NO: 27; (q) the VH region amino acid sequence comprises SEQ ID NO: 28 and the VL region amino acid sequence comprises SEQ ID NO: 29; (r) the VH region amino acid sequence comprises SEQ ID NO: 30 and the VL region amino acid sequence comprises SEQ ID NO: 31; (s) the VH region amino acid sequence comprises SEQ ID NO: 32 and the VL region amino acid sequence comprises SEQ ID NO: 33; (t) the VH region amino acid sequence comprises SEQ ID NO: 34 and the VL region amino acid sequence comprises SEQ ID NO: 35; (u) the VH region amino acid sequence comprises SEQ ID NO: 36 and the VL region amino acid sequence comprises SEQ ID NO: 37; (v) the VH region amino acid sequence comprises SEQ ID NO: 38 and the VL region amino acid sequence comprises SEQ ID NO: 39; (w) the VH region amino acid sequence comprises SEQ ID NO: 40 and the VL region amino acid sequence comprises SEQ ID NO: 41; (x) the VH region amino acid sequence comprises SEQ ID NO: 42 and the VL region amino acid sequence comprises SEQ ID NO: 43; (y) the VH region amino acid sequence comprises SEQ ID NO: 44 and the VL region amino acid sequence comprises SEQ ID NO: 45; (z) the VH region amino acid sequence comprises SEQ ID NO: 46 and the VL region amino acid sequence comprises SEQ ID NO: 47; (aa) the VH region amino acid sequence comprises SEQ ID NO: 48 and the VL region amino acid sequence comprises SEQ ID NO: 49; (bb) the VH region amino acid sequence comprises SEQ ID NO: 50 and the VL region amino acid sequence comprises SEQ ID NO: 51; or (cc) the VH region amino acid sequence comprises SEQ ID NO: 52 and the VL region amino acid sequence comprises SEQ ID NO: 53.

In some embodiments, the antibody or antigen-binding portion is human or chimeric.

In some embodiments, the antibody or antigen-binding portion comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region is immunologically inert. In some embodiments, the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A, L235A and G237A or a wild-type human IgG2 constant region, wherein numbering is according to the EU index as in Kabat. In some embodiments, the immunoglobulin constant region comprises any one of SEQ ID NOs: 145-149.

In some embodiments, the antibody or antigen-binding portion is an Fab, an Fab′, an F(ab′)2, an Fv, an scFv, a maxibody, a minibody, a diabody, a triabody, a tetrabody, or a bis-scFv.

In some embodiments, the antibody is monoclonal.

In some embodiments, the antibody or antigen-binding portion is tetrameric antibody, tetravalent or multispecific. In some embodiments, the antibody or antigen-binding portion is a bispecific antibody or antigen-binding portion that binds specifically to a first antigen and a second antigen, wherein the first antigen is VISTA and the second antigen is not VISTA.

Further provided herein is an immunoconjugate comprising an anti-VISTA antibody or an antigen-binding portion disclosed herein, linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic peptide, a therapeutic nucleic acid, an anti-angiogenic agent, an anti-proliferative agent, or a pro-apoptotic agent.

Provided herein is a pharmaceutical composition comprising an anti-VISTA antibody, an antigen-binding portion or an immunoconjugate disclosed herein, and a pharmaceutically acceptable carrier, diluent or excipient.

Further provided herein is a nucleic acid molecule encoding (a) the VH region amino acid sequence; (b) the VL region amino acid sequence; or (c) both the VH and the VL region amino acid sequences of an anti-VISTA antibody or an antigen-binding portion disclosed herein.

Provided herein is an expression vector comprising a nucleic acid molecule disclosed herein. Also provided herein is a recombinant host cell comprising a nucleic acid molecule or an expression vector disclosed herein.

Further provided herein is a method of producing an anti-VISTA antibody or an antigen-binding portion thereof, the method comprising: culturing a recombinant host cell comprising an expression vector disclosed herein under conditions whereby the nucleic acid molecule is expressed, thereby producing the antibody or antigen-binding portion; and isolating the antibody or antigen-binding portion from the host cell or culture.

Provided herein is a method for inducing an immune response in a subject, comprising administering to the subject a therapeutically effective amount of an anti-VISTA antibody or antigen-binding portion, an immunoconjugate, or a pharmaceutical composition disclosed herein. In some embodiments, the immune response is an immune response against cancer cells. In some embodiments, the immune response is antibody-dependent cellular cytotoxicity, complement-dependent cytotoxicity or antibody-dependent cellular phagocytosis.

Provided herein is a method for treating or preventing a cancer in a subject, comprising administering to the subject a therapeutically effective amount of an anti-VISTA antibody or antigen-binding portion, an immunoconjugate, or a pharmaceutical composition disclosed herein. In some embodiments, the cancer is a hematologic malignancy or an epithelial cancer. In some embodiments, the cancer is squamous cell carcinoma of head and neck, melanoma, non-small cell lung cancer or colon cancer. In some embodiments, the cancer is acute myeloid leukemia (AML).

In some embodiments, the subject overexpresses VISTA. In some embodiments, the subject overexpresses VISTA on immune cells. In some embodiments, the immune cells are tumor-infiltrating immune cells. In some embodiments, the immune cells are myeloid cells or monocytes.

In some embodiments, a method disclosed herein further comprises administering to the subject an inhibitor of PD-1 or an inhibitor of PD-L1. In some embodiments, the inhibitor of PD-1 is an anti-PD-1 antibody or antigen-binding portion thereof. In some embodiments, the inhibitor of PD-L1 is an anti-PD-L1 antibody or antigen-binding portion thereof.

Provided herein is an anti-VISTA antibody or antigen-binding portion, an immunoconjugate, or a pharmaceutical composition disclosed herein, for use as a medicament.

Provided herein is a method for treating a tumor in a subject, the method comprising:
(a) obtaining a VISTA:PSGL-1 proximity score for a tumor sample from the subject; wherein the VISTA:PSGL-1 proximity score is been assigned to the tumor sample by a process which comprises:
(i) obtaining an image of tissue that has been removed from the tumor sample and immunohistochemically stained for VISTA and PSGL-1 expression in a manner that allows stained VISTA cells to be distinguished from stained PSGL-1 cells;
(ii) defining in the image one or more regions of interest (ROIs) that comprises neoplastic cells and associated stroma, wherein substantially all of the neoplastic cells and associated stroma in the image are contained in the defined ROIs;
(iii) randomly creating across each defined ROI a plurality of subregions of substantially the same shape and size, wherein each of the subregions defines an area that is large enough to include a spatially proximal pair of a stained VISTA cell and a stained PSGL-1 cell and small enough to exclude pairs of stained VISTA and PSGL-1 cells that are not spatially proximal; and
(iv) calculating the percent of all of the subregions that are positive for both stained VISTA cells and stained PSGL-1 cells to generate the VISTA:PSGL-1 proximity score for the tumor sample,
(b) comparing the VISTA:PSGL-1 proximity score for the tumor sample with a threshold VISTA:PSGL-1 proximity score,
(c) classifying the tumor as biomarker positive or biomarker negative, wherein if the proximity score for the tumor sample is equal to or greater than the threshold proximity score, then the tumor is classified as positive for VISTA:PSGL-1 proximity biomarker, and if the obtained score is less than the threshold score, then the tumor is classified as negative for the VISTA:PSGL-1 proximity biomarker, and
(d) administering to the subject a therapeutically effective amount of an anti-VISTA antibody or antigen-binding portion, an immunoconjugate, or a pharmaceutical composition disclosed herein if the tumor is positive for the VISTA:PSGL-1 proximity biomarker.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a schematic of strategy implemented for primary selection of pH-dependent anti-VISTA antibodies. Round of selection is represented by “R”. MACS=Magnetic Activated Cell Sorting. FACS=Fluorescence Activated Cell Sorting.

FIG. 2 depicts a schematic of steps undertaken for discovery of parental anti-VISTA antibodies.

FIG. 3 depicts results from studies of binding characteristics of an exemplary anti-VISTA antibody. Avidity and monovalent affinity measurements at pH 7.4 and pH 6.0 were measured by Octet. Binding profiles of anti-VISTA mAb on CHO-VISTA (VISTA overexpressed) cells and KASUMI-3 (endogenous VISTA) cells are shown. FOB=Fold over background.

FIG. 4A-FIG. 4C depict results from studies of inhibitory characteristics of anti-VISTA antibodies. VISTA-Dextramer-PE interacts with PSGL-1 on primary T-cells at pH 6.0 (FIG. 4A). Anti-VISTA mAb #2 inhibits VISTA:PSGL-1 interaction on CD4 and CD8 T-cells (FIG. 4B). Top 8 anti-VISTA mAbs ranked by PSGL-1:VISTA competition ELISA are shown (FIG. 4C).

FIG. 5A-FIG. 5H depict results from studies of MC38 tumor growth inhibition in human VISTA knock-in mice. Black line (IgG CTRL human & rat), dark gray line (IgG CTRL human & rat anti-mPD-1; light gray line (rat anti-mPD-1 & anti-VISTA). CR=Complete response.

FIG. 6 depicts a schematic of steps undertaken for lead-optimization of top 8 parental anti-VISTA antibodies.

FIG. 7A-FIG. 7D depict results from studies characterizing optimized progeny anti-VISTA antibodies. Binding profile of two representative anti-VISTA parental antibodies and their respective progeny at pH 6.0 and pH 7.4 to KASUMI-3 cells (FIG. 7A). Binding profile (Fab on CHO-VISTA cells), inhibitory function and SPR characterization of SNS-101 (FIG. 7B, FIG. 7C and FIG. 7D). For SPR, monomeric VISTA at identical concentrations was passed over antibody immobilized on a Protein A surface at pH 6.0 or 7.4. RU=response units; N.B.=Non-binding; FOB=Fold over background.

FIG. 8A-FIG. 8B depict results of an in vivo test of activity of an anti-VISTA antibody. FIG. 8A shows effects on tumor volume over time. FIG. 8B shows effects on percent survival over time. Black line (IgG CTRL human & rat), dark gray line (IgG CTRL human & rat anti-mPD-1; light gray line (rat anti-mPD-1 & anti-VISTA).

FIG. 9A-FIG. 9L depict images and results of data analysis from a multiplexed immunohistochemistry assay to quantify degree of spatial proximity for VISTA+ cells and PSGL-1+ cells. FIG. 9A: 5VISTA_1 Tonsil (QC19-14). FIG. 9B: 5VISTA_1 Tonsil (QC19-14). FIG. 9C: 5VISTA_1 H&N (010-001). FIG. 9D: H&N (HN483a) Core C6,R7. FIG. 9E, FIG. 9F: Spatial Analysis H&N (HN83a) Core C6,R7. FIG. 9G: H&N (HN483a) Core C5,R4. FIG. 9H, FIG. 9I: Spatial Analysis H&N (HN83a) Core C5,R4. FIG. 9J: BrCA (BR1202a) Core C4,R9. FIG. 9K: BrCA (BR1202a) Core C9,R5. FIG. 9L: CRC (BC05012a) Core C5,R4.

FIG. 10A-FIG. 10E depict results from a cytokine release profile assay of SNS-101 in an ex vivo system that mimics human blood circulation. FIG. 10A: IFN-γ. FIG. 10B: IL-6. FIG. 10C: IL-8. FIG. 10D: TNF-α. FIG. 10E: IL-2. JNJ: variable region JNJ-61610588 antibody cloned onto human IgG1 backbone.

FIG. 10F-FIG. 10I depict results from a flow cytometry analysis of SNS-101 activation of human monocytes and natural killer (NK) cells. Proportions of CD83+ monocytes (FIG. 10F), CD83 MFI (median) (FIG. 10G), CD69+NK cells (FIG. 10H) and CD69 MFI (median) (FIG. 10I) are shown. JNJ: variable region JNJ-61610588 antibody cloned onto human IgG1 backbone. MFI: Median Fluorescence Intensity.

FIG. 11A-FIG. 11B depict results of pharmacokinetic (PK) profiling of the pH-sensitive anti-VISTA antibody SNS-101 in cynomolgus monkeys. FIG. 11A shows the PK profiles of SNS-101, which demonstrates linear kinetic elimination, and non-pH-selective antibody h26A, which demonstrates TMDD and rapid clearance). Analyses of data from FIG. 11A provide plasma CL, T_{1/2λ} and V_{SS} of SNS-101 over the doses of 1, 10, and 100 mg/kg as shown in FIG. 11B.

FIG. 12A-FIG. 12C depict results of a test in a MC-38 model in human VISTA knock-in mice where SNS-101 demonstrates strong combinatorial activity with an anti-PD-1 antibody and increases the level of CD8+ T-cells. Effects on tumor volume are shown in FIG. 12A. FIG. 12B shows the frequency of CD8+ cells as determined in the single, live, CD45+ population by analytical flow cytometry analysis. FIG. 12C depicts a heat-map of relative cytokine levels across different treatment groups from the study. The following treatment groups were tested. 1: Isotype Control. 2: aPD-1 at 1 mg/kg. 3: SNS-101 at 10 mg/kg. 4: SNS-101 at 30 mg/kg. 5: aPD-1 at 1 mg/kg+SNS-101 at 10 mg/kg. 6: αPD-1 at 1 mg/kg+SNS-101 at 30 mg/kg. The heatmap was drawn with scaled mean cytokine levels of groups (z-scores of log-transformed data). The cytokines were arranged by hierarchical clustering on Euclidean distance of cytokine profiles.

FIG. 12D-FIG. 12H depict results of a test in a MB49 syngeneic tumor model in human VISTA knock-in mice. Mice were treated with either anti-murine PD-1 antibody (RMP1-14) alone at 1 mg/kg and 5 mg/kg or in combination with SNS-101-m2 at 3 mg/kg, 10 mg/kg and 30 mg/kg, respectively. Effects on tumor volumes of individual mice (FIG. 12D and FIG. 12E) and aggregated treatment groups (FIG. 12F and FIG. 12G) are shown. The groups were treated with (1) IgG control (CTRL) mouse & rat, (2) IgG CTRL mouse & rat anti-mPD-1, or (3) rat anti-mPD-1 & anti-VISTA. FIG. 12H depicts a heat-map of relative cytokine levels across different treatment groups from the study. The following treatment groups were tested. 1: Isotype Control. 2: aPD-1 at 1 mg/kg. 3: aPD-1 at 5 mg/kg. 4: SNS-101 at 3 mg/kg. 5: aPD-1 at 1 mg/kg+SNS-101 at 3 mg/kg. 6: αPD-1 at 5 mg/kg+SNS-101 at 3 mg/kg. The heatmap was drawn with scaled mean cytokine levels of groups (z-scores of log-transformed data). The cytokines were arranged by hierarchical clustering on Euclidean distance of cytokine profiles.

FIG. 13 depicts results from a cytokine release profile assay of SNS-101 in an in vivo cytokine release syndrome mouse model. JNJ: JNJ: variable region JNJ-61610588 antibody cloned onto human IgG1 backbone. OKT3: positive control anti-CD3 antibody. Dotted line=detection limit.

FIG. 14A-FIG. 14B depict images of the putative SNS-101 (gray):VISTA binding interface (FIG. 14A; defined by peptide array binding (PepScan)) to the position of 5 key His residues involved in PSGL-1 (dark gray) binding, as well as residues proposed to be involved with binding of putative partners VSIG-3 (light gray) and LRIG-1 (medium gray) (FIG. 14B).

FIG. 14C-FIG. 14G depict the crystal structure of a hVISTA:SNS101_Fab complex. hVISTA is represented in molecular surface (FIG. 14D, FIG. 14F) with SNS101_Fab is represented in ribbon cartoon (FIG. 14C, FIG. 14E), respectively. The blocked surface of VISTA in the presence of SNS-101 is indicated in light gray shade whereas the unblocked portion in dark grey shade. The dark and light shade of ribbon indicate the light and heavy chain of SNS-101_Fab, respectively. The blocked surface was created with all amino acids of hVISTA that are within 4 A (FIG. 14C, FIG. 14D) or 6A (FIG. 14E, FIG. 14F) away from any part of SNS-101. FIG. 14G depicts PSGL-1, VSIG-3 and LRIG-1 interacting amino acids highlighted on the hVISTA structure. PSGL-1: Black (His153/154/155/98/100). VSIG-3: Dark gray (Phe94/Gln95/Arg86). LRIG-1: Medium gray (Thr82/Arg87). hVISTA—light gray.

FIG. 15 depicts results from competition experiments with dilutions of SNS-101 or isotype control, other potential VISTA binding partners and biotinylated hVISTA-Fc.

### DETAILED DESCRIPTION

Provided herein are anti-VISTA antibodies and therapeutic uses of such antibodies. The antibodies disclosed herein inhibit the interaction of VISTA with its receptor PSGL-1 (P-selectin glycoprotein ligand-1).

V-domain immunoglobulin suppressor of T cell activation (VISTA; also known as c10orf54, VSIR, SISP1, B7-H5, PD-1H, DD1α, Gi24, and Dies 1) is a type I transmembrane protein consisting of a single N-terminal immunoglobulin (Ig) V-domain, a stalk of approximately 30 amino acids (aa), a transmembrane domain, and an approximately 95-aa cytoplasmic tail. VISTA is an inhibitory B7 family immune-checkpoint protein that is a negative regulator of T-cell function. VISTA is constitutively expressed on multiple immune cell types.

As VISTA is highly expressed on myeloid cells, including those in the blood, antibodies binding VISTA at physiological pH 7.4 (VISTA^{ppH}) can result in rapid elimination from circulation through target-mediated drug disposition (TMDD), making efficacious drug occupancy levels difficult to reach and potentially narrowing the therapeutic window. Provided herein are anti-VISTA antibodies that exhibit pH-sensitive inhibitory VISTA engagement. Such antibodies have enhanced pharmacokinetic properties that enable less-frequent dosing. These antibodies also provide a wider therapeutic window. In some aspects, anti-VISTA antibodies provided herein are pH-selective, inhibitory, high-affinity and cross-reactive with cynomolgus VISTA. In some aspects, anti-VISTA antibodies provided herein exhibit combinability with anti-PD-1 inhibitors, leading to superior anti-tumor activity. Because VISTA is implicated in cancer resistance to inhibitors of PD-1/PD-L1, anti-VISTA antibodies that are compatible with PD-1/PD-L1 inhibition are advantageous. In some embodiments, antibodies provided herein have a reduced risk for inducing cytokine release syndrome (CRS). Antibodies provided herein can prevent the on-target, off-tumor VISTA binding that may drive CRS in human patients.

### Antibodies

Provided herein are antibodies and antigen-binding portions thereof that specifically bind VISTA. Development of therapeutics targeting VISTA has been hampered by the presence of a pharmacological “sink” due to the high expression of VISTA on hematopoietic (blood) cells. In addition, it has been shown that VISTA binds its receptor on T-cells (PSGL-1) at sub-physiologic pH (˜6). Anti-VISTA antibodies and antigen-binding portions provided herewith exhibit pH-selective binding behavior to VISTA, thus mitigating TMDD. In some embodiments, antibodies provided herein bind VISTA only at the acidic pH found in the tumor microenvironment (pH ˜6), which is lower than the blood pH levels (pH 7.4), enabling the selective inhibition of VISTA within a tumor.

Antibodies and antigen-binding portions disclosed herein specifically bind human VISTA. In some embodiments, antibodies and antigen-binding portions may cross-react with VISTA from species other than human, for example, cynomolgus monkey (Macaca fascicularis) VISTA. In some embodiments, an antibody may be specific for only human VISTA and may exhibit no non-human cross-reactivity. Exemplary amino acid sequences of human and cynomolgus VISTA are provided in Table 31.

The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable region (abbreviated herein as VH region) and a heavy chain constant region. The heavy chain constant region comprises three domains, CH1, CH2 and CH3. Each light chain comprises a light chain variable region (abbreviated herein as VL region) and a light chain constant region. The light chain constant region comprises one domain, CL. The VH and VL regions can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kabat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH) and a VL. Generally, IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

The term “antigen-binding portion” or “antigen-binding fragment” of an antibody (or “antibody portion” or “antibody fragment”), as used herein, refers to one or more fragments of an antibody that retain the ability to specifically bind to an antigen (e.g., VISTA). It has been shown that the antigen-binding function of an antibody can be performed by portions or fragments of a full-length antibody. Examples of binding portions encompassed within the term “antigen binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (domain antibody) fragment (Ward et al., (1989) Nature 341:544-546; WO 90/05144 A1, each herein incorporated by reference in its entirety), which comprises a single variable domain; and (vi) an isolated complementarity determining region (CDR). The disclosure also encompasses a Fab′ fragment. Fab′ fragments can be formed by the reduction of F(ab′)2 fragments. Fab′ is derived from F(ab′)2; therefore, it may contain a small portion of Fc. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv). See e.g., Bird et al. (1988) Science 242:423-426; Huston et al. (1988) Proc. Natl. Acad. Sci. USA 85:5879-5883. Such single chain antibodies are also intended to be encompassed within the term “antigen-binding portion” of an antibody. In some embodiments, scFv molecules may be incorporated into a fusion protein. In some embodiments, provided herein is a single chain camelid antibody. In some embodiments, provided herein is a shark heavy chain antibody (V-NAR). See, English et al. (2020) Antibody Therapeutics, 3(1):1-9. Examples of antigen-binding portions are known in the art (Kontermann and Dubel eds., Antibody Engineering (2001) Springer-Verlag. New York. 790 pp.). In some embodiments, provided herein is a single domain antibody. In general, the term “antibody” when used herein encompasses an “antibody portion”. An antibody portion generally retains the antigen-binding properties of a full-length antibody.

Antibodies and antibody portions provided herein may be in multispecific (e.g., bispecific or trispecific) formats. Such multispecific molecules specifically bind to two or more different molecular targets or epitopes. In some embodiments, an antibody or antigen-binding portion provided herein is tetrameric, tetravalent or multispecific. In some embodiments, an antibody or an antigen-binding portion is a bispecific molecule that binds specifically to a first antigen and a second antigen, wherein the first antigen is VISTA and the second antigen is not VISTA. In some embodiments, an antibody or an antigen-binding portion is a diabody. Diabodies are bivalent, bispecific antibodies in which VH and VL domains are expressed on a single polypeptide chain but using a linker that is too short to allow for pairing between the two domains on the same chain, thereby forcing the domains to pair with complementary domains of another chain and creating two antigen-binding sites (see e.g., Holliger et al. (1993) Proc. Natl. Acad. Sci. USA 90:6444-6448; Poljak et al. (1994) Structure 2:1121-1123). In some embodiments, an antibody or an antigen-binding portion is a triabody, a tetrabody, a bis-scFv or a tandem scFv. In some embodiments, an antibody or an antigen-binding portion is a dual affinity re-targeting protein.

In some embodiments, an anti-VISTA antibody or antigen-binding portion disclosed herein is an Fab, an Fab′, an F(ab′)2, an Fv, an scFv, a maxibody, a minibody, a diabody, a triabody, a tetrabody, or a bis-scFv.

As used herein, the terms “immunological binding” and “immunological binding properties” refer to the non-covalent interactions of the type which occur between an immunoglobulin molecule (e.g., antibody or antigen-binding portion thereof) and an antigen for which the immunoglobulin is specific. The strength, or affinity, of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind VISTA when the equilibrium binding constant (K_{d}) is ≤10 μM, preferably ≤10 nM, more preferably ≤10 nM, and most preferably ≤100 pM to about 1 pM, as measured by assays such as radioligand binding assays or similar assays known to those skilled in the art. In some embodiments, the K_{d} of an antibody is measured by using surface plasmon resonance (SPR), typically using a biosensor system such as a Biacore® system. In some embodiments, the K_{d} of an antibody is measured by a platform based on bio-layer interferometry technology, such as the Octet system (Creative Biolabs, Shirley, N.Y.).

In some embodiments, an anti-VISTA antibody or antigen-binding portion provided herein is monovalent or bivalent and comprises a single or double chain. Functionally, the binding affinity of an antibody or antigen-binding portion may be within the range of 10⁻⁵M to 10⁻¹² M. For example, the binding affinity of an antibody or antigen-binding portion is from 10⁻⁶M to 10⁻¹² M, from 10⁻⁷ M to 10⁻¹² M, from 10⁻⁸ M to 10⁻¹² M, from 10⁻⁹ M to 10⁻¹² M, from 10⁻⁵M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10 M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵M to 10⁻⁸ M, from 10⁻⁶ M to 10⁻⁸M, from 10⁻⁷ M to 10⁻⁸ M, from 10⁻⁵M to 10⁻⁷ M, from 10⁻⁶ M to 10⁻⁷M or from 10⁻⁵ M to 10⁻⁶ M.

In some embodiments, the monovalent affinity (K_{d}) at pH 6.0 of an anti-VISTA antibody or antigen-binding portion provided herein is less than 9 nM, 8 nM, 7 nM, 6 nM, 5 nM, 4 nM, 3 nM, 2 nM, 1 nM, 0.5 nM or 0.25 nM, as measured by SPR. In some embodiments, the monovalent affinity (K_{d}) at pH 6.0 of an anti-VISTA antibody or antigen-binding portion provided herein is from about 0.1 nM to about 0.5 nM, from about 0.2 nM to about 0.4 nM, or from about 0.2 nM to about 0.3 nM, as measured by SPR.

In some embodiments, the monovalent affinity (K_{d}) at physiological pH (pH 7.0-7.4) of an anti-VISTA antibody or antigen-binding portion provided herein is greater than 100 nM, as measured by SPR. In some embodiments, no specific binding to VISTA is detected at physiological pH (pH 7.0-7.4) by an anti-VISTA antibody or antigen-binding portion provided herein, as measured by SPR.

In some embodiments, the monovalent affinity (K_{d}) at pH 6.0 of an anti-VISTA antibody or antigen-binding portion provided herein is from about 0.2 nM to about 0.3 nM, and the monovalent affinity (K_{d}) at physiological pH (pH 7.0-7.4) of the antibody or antigen-binding portion is greater than 100 nM, as measured by SPR.

In some embodiments, the monovalent affinity (K_{d}) at pH 6.0 of an anti-VISTA antibody or antigen-binding portion provided herein is at least 100-fold lower, at least 200-fold lower, at least 300-fold lower, at least 400-fold lower or at least 500-fold lower than the monovalent affinity (K_{d}) of the antibody or antigen-binding portion at physiological pH (pH 7.0-7.4).

Provided herein is an anti-VISTA antibody or an antigen-binding portion thereof, wherein the antibody or antigen-binding portion cross-competes for binding to VISTA with antibody 55873, 67373, 67374, 67375, 67376, 67377, 67378, 67379, 67380, 67381, 67382, 55902, 67414, 67415, 67416, 67417, 67418, 67419, 67420, 67422, 67423, 67424, 55872, 55879, 55880, 55899, 55901, 55932 or 55934, or an antibody that comprises one or more amino acid sequences of antibody 55873, 67373, 67374, 67375, 67376, 67377, 67378, 67379, 67380, 67381, 67382, 55902, 67414, 67415, 67416, 67417, 67418, 67419, 67420, 67422, 67423, 67424, 55872, 55879, 55880, 55899, 55901, 55932 or 55934 (see Tables 1-29).

The terms “cross-compete”, “cross-competition”, “cross-block”, “cross-blocked” and “cross-blocking” are used interchangeably herein to mean the ability of an antibody or an antigen-binding portion thereof to interfere with the binding directly or indirectly through allosteric modulation of the anti-VISTA antibodies of the disclosure to the target VISTA (e.g., human VISTA). The extent to which an antibody or portion thereof is able to interfere with the binding of another to the target, and therefore whether it can be said to cross-block or cross-compete, can be determined using competition binding assays. One example of a binding competition assay is Homogeneous Time Resolved Fluorescence (HTRF). One particularly suitable quantitative cross-competition assay uses a FACS- or an Alphascreen-based approach to measure competition between the labelled (e.g., His-tagged, biotinylated or radioactive labelled) antibody or portion thereof and the other antibody or portion thereof in terms of their binding to the target. In general, a cross-competing antibody or portion thereof is, for example, one which will bind to the target in the cross-competition assay such that, during the assay and in the presence of a second antibody or portion thereof, the recorded displacement of the immunoglobulin single variable domain or polypeptide according to the invention is up to 100% (e.g. in a FACS-based competition assay) of the maximum theoretical displacement (e.g. displacement by cold (e.g., unlabeled) antibody or fragment thereof that needs to be cross-blocked) by the potentially cross-blocking antibody or fragment thereof that is present in a given amount. In some embodiments, cross-competing antibodies or portions thereof have a recorded displacement that is between 10% and 100%, or between 50% and 100%.

Provided herein is an anti-VISTA antibody or an antigen-binding portion thereof, wherein the antibody or antigen-binding portion cross-competes for binding to VISTA with an antibody comprising a VH region and a VL region, wherein: (a) the VH region amino acid sequence comprises a HCDR1 comprising SEQ ID NO: 54, a HCDR2 comprising SEQ ID NO: 55 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (b) the VH region amino acid sequence comprises a HCDR1 comprising SEQ ID NO: 60, a HCDR2 comprising SEQ ID NO: 61 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (c) the VH region amino acid sequence comprises a HCDR1 comprising SEQ ID NO: 62, a HCDR2 comprising SEQ ID NO: 63 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (d) the VH region amino acid sequence comprises a HCDR1 comprising SEQ ID NO: 64, a HCDR2 comprising SEQ ID NO: 65 and a HCDR3 comprising SEQ ID NO: 66; and the VL region amino acid sequence comprises a LCDR1 comprising SEQ ID NO: 57, a LCDR2 comprising SEQ ID NO: 67 and a LCDR3 comprising SEQ ID NO: 68; (e) the VH region amino acid sequence comprises a HCDR1 comprising SEQ ID NO: 69, a HCDR2 comprising SEQ ID NO: 70 and a HCDR3 comprising SEQ ID NO: 56; and the VL region amino acid sequence comprises a LCDR1 comprising SEQ ID NO: 57, a LCDR2 comprising SEQ ID NO: 67 and a LCDR3 comprising SEQ ID NO: 71; (f) the VH region amino acid sequence comprises a HCDR1 comprising SEQ ID NO: 60, a HCDR2 comprising SEQ ID NO: 61 and a HCDR3 comprising SEQ ID NO: 72; and the VL region amino acid sequence comprises a LCDR1 comprising SEQ ID NO: 57, a LCDR2 comprising SEQ ID NO: 58 and a LCDR3 comprising SEQ ID NO: 68; (g) the VH region amino acid sequence comprises a HCDR1 comprising SEQ ID NO: 73, a HCDR2 comprising SEQ ID NO: 65 and a HCDR3 comprising SEQ ID NO: 74; and the VL region amino acid sequence comprises a LCDR1 comprising SEQ ID NO: 57, a LCDR2 comprising SEQ ID NO: 58 and a LCDR3 comprising SEQ ID NO: 71; (h) the VH region amino acid sequence comprises a HCDR1 comprising SEQ ID NO: 64, a HCDR2 comprising SEQ ID NO: 75 and a HCDR3 comprising SEQ ID NO: 66; and the VL region amino acid sequence comprises a LCDR1 comprising SEQ ID NO: 57, a LCDR2 comprising SEQ ID NO: 76 and a LCDR3 comprising SEQ ID NO: 68; (i) the VH region amino acid sequence comprises a HCDR1 comprising SEQ ID NO: 64, a HCDR2 comprising SEQ ID NO: 65 and a HCDR3 comprising SEQ ID NO: 77; and the VL region amino acid sequence comprises a LCDR1 comprising SEQ ID NO: 57, a LCDR2 comprising SEQ ID NO: 78 and a LCDR3 comprising SEQ ID NO: 68; (j) the VH region amino acid sequence comprises a HCDR1 comprising SEQ ID NO: 60, a HCDR2 comprising SEQ ID NO: 75 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 68; (k) the VH region amino acid sequence comprises a HCDR1 comprising SEQ ID NO: 54, a HCDR2 comprising SEQ ID NO: 65 and a HCDR3 comprising SEQ ID NO: 79; and the VL region amino acid sequence comprises a LCDR1 comprising SEQ ID NO: 80, a LCDR2 comprising SEQ ID NO: 58 and a LCDR3 comprising SEQ ID NO: 68; (1) the VH region amino acid sequence comprises a HCDR1 comprising SEQ ID NO: 81, a HCDR2 comprising SEQ ID NO: 82 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (m) the VH region amino acid sequence comprises a HCDR1 comprising SEQ ID NO: 87, a HCDR2 comprising SEQ ID NO: 88 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (n) the VH region amino acid sequence comprises a HCDR1 comprising SEQ ID NO: 89, a HCDR2 comprising SEQ ID NO: 90 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (o) the VH region amino acid sequence comprises a HCDR1 comprising SEQ ID NO: 91, a HCDR2 comprising SEQ ID NO: 92 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (p) the VH region amino acid sequence comprises a HCDR1 comprising SEQ ID NO: 93, a HCDR2 comprising SEQ ID NO: 94 and a HCDR3 comprising SEQ ID NO: 95; and the VL region amino acid sequence comprises a LCDR1 comprising SEQ ID NO: 96, a LCDR2 comprising SEQ ID NO: 85 and a LCDR3 comprising SEQ ID NO: 97; (q) the VH region amino acid sequence comprises a HCDR1 comprising SEQ ID NO: 98, a HCDR2 comprising SEQ ID NO: 99 and a HCDR3 comprising SEQ ID NO: 100; and the VL region amino acid sequence comprises a LCDR1 comprising SEQ ID NO: 96, a LCDR2 comprising SEQ ID NO: 101 and a LCDR3 comprising SEQ ID NO: 102; (r) the VH region amino acid sequence comprises a HCDR1 comprising SEQ ID NO: 103, a HCDR2 comprising SEQ ID NO: 104 and a HCDR3 comprising SEQ ID NO: 105; and the VL region amino acid sequence comprises a LCDR1 comprising SEQ ID NO: 96, a LCDR2 comprising SEQ ID NO: 106 and a LCDR3 comprising SEQ ID NO: 107; (s) the VH region amino acid sequence comprises a HCDR1 comprising SEQ ID NO: 108, a HCDR2 comprising SEQ ID NO: 94 and a HCDR3 comprising SEQ ID NO: 105; and the VL region amino acid sequence comprises a LCDR1 comprising SEQ ID NO: 109, a LCDR2 comprising SEQ ID NO: 85 and a LCDR3 comprising SEQ ID NO: 110; (t) the VH region amino acid sequence comprises a HCDR1 comprising SEQ ID NO: 111, a HCDR2 comprising SEQ ID NO: 94 and a HCDR3 comprising SEQ ID NO: 112; and the VL region amino acid sequence comprises a LCDR1 comprising SEQ ID NO: 96, a LCDR2 comprising SEQ ID NO: 85 and a LCDR3 comprising SEQ ID NO: 113; (u) the VH region amino acid sequence comprises a HCDR1 comprising SEQ ID NO: 87, a HCDR2 comprising SEQ ID NO: 94 and a HCDR3 comprising SEQ ID NO: 114; and the VL region amino acid sequence comprises a LCDR1 comprising SEQ ID NO: 115, a LCDR2 comprising SEQ ID NO: 85 and a LCDR3 comprising SEQ ID NO: 116; (v) the VH region amino acid sequence comprises a HCDR1 comprising SEQ ID NO: 93, a HCDR2 comprising SEQ ID NO: 90 and a HCDR3 comprising SEQ ID NO: 117; and the VL region amino acid sequence comprises a LCDR1 comprising SEQ ID NO: 118, a LCDR2 comprising SEQ ID NO: 85 and a LCDR3 comprising SEQ ID NO: 86; (w) the VH region amino acid sequence comprises a HCDR1 comprising SEQ ID NO: 119, a HCDR2 comprising SEQ ID NO: 120 and a HCDR3 comprising SEQ ID NO: 121; and the VL region amino acid sequence comprises a LCDR1 comprising SEQ ID NO: 57, a LCDR2 comprising SEQ ID NO: 58 and a LCDR3 comprising SEQ ID NO: 122; (x) the VH region amino acid sequence comprises a HCDR1 comprising SEQ ID NO: 123, a HCDR2 comprising SEQ ID NO: 124 and a HCDR3 comprising SEQ ID NO: 125; and the VL region amino acid sequence comprises a LCDR1 comprising SEQ ID NO: 126, a LCDR2 comprising SEQ ID NO: 127 and a LCDR3 comprising SEQ ID NO: 128; (y) the VH region amino acid sequence comprises a HCDR1 comprising SEQ ID NO: 123, a HCDR2 comprising SEQ ID NO: 124 and a HCDR3 comprising SEQ ID NO: 129; and the VL region amino acid sequence comprises a LCDR1 comprising SEQ ID NO: 57, a LCDR2 comprising SEQ ID NO: 58 and a LCDR3 comprising SEQ ID NO: 130; (z) the VH region amino acid sequence comprises a HCDR1 comprising SEQ ID NO: 131, a HCDR2 comprising SEQ ID NO: 132 and a HCDR3 comprising SEQ ID NO: 133; and the VL region amino acid sequence comprises a LCDR1 comprising SEQ ID NO: 126, a LCDR2 comprising SEQ ID NO: 134 and a LCDR3 comprising SEQ ID NO: 135; (aa) the VH region amino acid sequence comprises a HCDR1 comprising SEQ ID NO: 131, a HCDR2 comprising SEQ ID NO: 132 and a HCDR3 comprising SEQ ID NO: 136; and the VL region amino acid sequence comprises a LCDR1 comprising SEQ ID NO: 57, a LCDR2 comprising SEQ ID NO: 58 and a LCDR3 comprising SEQ ID NO: 137; (bb) the VH region amino acid sequence comprises a HCDR1 comprising SEQ ID NO: 54, a HCDR2 comprising SEQ ID NO: 138 and a HCDR3 comprising SEQ ID NO: 139; and the VL region amino acid sequence comprises a LCDR1 comprising SEQ ID NO: 57, a LCDR2 comprising SEQ ID NO: 58 and a LCDR3 comprising SEQ ID NO: 140; or (cc) the VH region amino acid sequence comprises a HCDR1 comprising SEQ ID NO: 81, a HCDR2 comprising SEQ ID NO: 82 and a HCDR3 comprising SEQ ID NO: 141; and the VL region amino acid sequence comprises a LCDR1 comprising SEQ ID NO: 142, a LCDR2 comprising SEQ ID NO: 143 and a LCDR3 comprising SEQ ID NO: 144.

Provided herein is an anti-VISTA antibody or an antigen-binding portion thereof, wherein the antibody or antigen-binding portion cross-competes for binding to VISTA with the antibody or antigen-binding portion comprising the sets of CDRs disclosed herein; and (a) binds specifically to human VISTA and cynomolgus VISTA; (b) has at least a 500-fold lower K_{d} at pH 6 than at physiological pH (pH 7.0-7.4); (c) inhibits the PSGL-1/VISTA interaction; (d) alleviates VISTA-induced checkpoint blockade; (e) induces tumor rejection in >40% of syngeneic mice when used as a monotherapy; and (f) induces tumor rejection in >90% of syngeneic mice when used in combination with an anti-PD-1 inhibitor.

The antibodies disclosed herein are anti-VISTA antagonist antibodies. As used herein, an “antagonist” or an “anti-VISTA antagonist antibody” (interchangeably termed “anti-VISTA antibody”) refers to an antibody which is able to bind to VISTA and inhibit VISTA biological activity and/or downstream pathway(s) mediated by VISTA signalling. An anti-VISTA antagonist antibody encompasses antibodies that can block, antagonize, suppress or reduce (including significantly) VISTA biological activity, including downstream pathways mediated by VISTA signalling, such as receptor binding and/or elicitation of a cellular response to VISTA. For the purposes of the present disclosure, it will be explicitly understood that the term “anti-VISTA antagonist antibody” encompass all the terms, titles, and functional states and characteristics whereby VISTA itself, and VISTA biological activity (including but not limited to its ability to negatively regulate T cells), or the consequences of the activity or biological activity, are substantially nullified, decreased, or neutralized in a meaningful degree.

Provided herein is an anti-VISTA antibody or an antigen-binding portion thereof that inhibits the PSGL-1/VISTA interaction. Provided herein is an anti-VISTA antibody or an antigen-binding portion thereof that inhibits the VSIG-3/VISTA interaction. Provided herein is an anti-VISTA antibody or an antigen-binding portion thereof that inhibits the VSIG-8/VISTA interaction. Provided herein is an anti-VISTA antibody or an antigen-binding portion thereof that inhibits the Syndecan-2/VISTA interaction. Provided herein is an anti-VISTA antibody or an antigen-binding portion thereof that inhibits the LRIG-1/VISTA interaction.

Further provided herein is an anti-VISTA antibody or an antigen-binding portion thereof that comprises one or more amino acid sequences of antibody 55873, 67373, 67374, 67375, 67376, 67377, 67378, 67379, 67380, 67381, 67382, 55902, 67414, 67415, 67416, 67417, 67418, 67419, 67420, 67422, 67423, 67424, 55872, 55879, 55880, 55899, 55901, 55932 or 55934. The combinations of VH region, VL region and CDR sequences forming these antibodies are provided in Tables 1-29. In some embodiments, the VH region sequence and/or the VL region sequence comprises a signal sequence (also known as a signal peptide) at the amino-terminus.

Provided herein is an anti-VISTA antibody or an antigen-binding portion thereof, wherein the antibody or antigen-binding portion comprises a heavy chain variable (VH) region and a light chain variable (VL) region wherein: (a) the VH region amino acid sequence comprises a HCDR1 comprising SEQ ID NO: 54, a HCDR2 comprising SEQ ID NO: 55 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (b) the VH region amino acid sequence comprises a HCDR1 comprising SEQ ID NO: 60, a HCDR2 comprising SEQ ID NO: 61 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (c) the VH region amino acid sequence comprises a HCDR1 comprising SEQ ID NO: 62, a HCDR2 comprising SEQ ID NO: 63 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (d) the VH region amino acid sequence comprises a HCDR1 comprising SEQ ID NO: 64, a HCDR2 comprising SEQ ID NO: 65 and a HCDR3 comprising SEQ ID NO: 66; and the VL region amino acid sequence comprises a LCDR1 comprising SEQ ID NO: 57, a LCDR2 comprising SEQ ID NO: 67 and a LCDR3 comprising SEQ ID NO: 68; (e) the VH region amino acid sequence comprises a HCDR1 comprising SEQ ID NO: 69, a HCDR2 comprising SEQ ID NO: 70 and a HCDR3 comprising SEQ ID NO: 56; and the VL region amino acid sequence comprises a LCDR1 comprising SEQ ID NO: 57, a LCDR2 comprising SEQ ID NO: 67 and a LCDR3 comprising SEQ ID NO: 71; (f) the VH region amino acid sequence comprises a HCDR1 comprising SEQ ID NO: 60, a HCDR2 comprising SEQ ID NO: 61 and a HCDR3 comprising SEQ ID NO: 72; and the VL region amino acid sequence comprises a LCDR1 comprising SEQ ID NO: 57, a LCDR2 comprising SEQ ID NO: 58 and a LCDR3 comprising SEQ ID NO: 68; (g) the VH region amino acid sequence comprises a HCDR1 comprising SEQ ID NO: 73, a HCDR2 comprising SEQ ID NO: 65 and a HCDR3 comprising SEQ ID NO: 74; and the VL region amino acid sequence comprises a LCDR1 comprising SEQ ID NO: 57, a LCDR2 comprising SEQ ID NO: 58 and a LCDR3 comprising SEQ ID NO: 71; (h) the VH region amino acid sequence comprises a HCDR1 comprising SEQ ID NO: 64, a HCDR2 comprising SEQ ID NO: 75 and a HCDR3 comprising SEQ ID NO: 66; and the VL region amino acid sequence comprises a LCDR1 comprising SEQ ID NO: 57, a LCDR2 comprising SEQ ID NO: 76 and a LCDR3 comprising SEQ ID NO: 68; (i) the VH region amino acid sequence comprises a HCDR1 comprising SEQ ID NO: 64, a HCDR2 comprising SEQ ID NO: 65 and a HCDR3 comprising SEQ ID NO: 77; and the VL region amino acid sequence comprises a LCDR1 comprising SEQ ID NO: 57, a LCDR2 comprising SEQ ID NO: 78 and a LCDR3 comprising SEQ ID NO: 68; (j) the VH region amino acid sequence comprises a HCDR1 comprising SEQ ID NO: 60, a HCDR2 comprising SEQ ID NO: 75 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 68; (k) the VH region amino acid sequence comprises a HCDR1 comprising SEQ ID NO: 54, a HCDR2 comprising SEQ ID NO: 65 and a HCDR3 comprising SEQ ID NO: 79; and the VL region amino acid sequence comprises a LCDR1 comprising SEQ ID NO: 80, a LCDR2 comprising SEQ ID NO: 58 and a LCDR3 comprising SEQ ID NO: 68; (1) the VH region amino acid sequence comprises a HCDR1 comprising SEQ ID NO: 81, a HCDR2 comprising SEQ ID NO: 82 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (m) the VH region amino acid sequence comprises a HCDR1 comprising SEQ ID NO: 87, a HCDR2 comprising SEQ ID NO: 88 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (n) the VH region amino acid sequence comprises a HCDR1 comprising SEQ ID NO: 89, a HCDR2 comprising SEQ ID NO: 90 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (o) the VH region amino acid sequence comprises a HCDR1 comprising SEQ ID NO: 91, a HCDR2 comprising SEQ ID NO: 92 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (p) the VH region amino acid sequence comprises a HCDR1 comprising SEQ ID NO: 93, a HCDR2 comprising SEQ ID NO: 94 and a HCDR3 comprising SEQ ID NO: 95; and the VL region amino acid sequence comprises a LCDR1 comprising SEQ ID NO: 96, a LCDR2 comprising SEQ ID NO: 85 and a LCDR3 comprising SEQ ID NO: 97; (q) the VH region amino acid sequence comprises a HCDR1 comprising SEQ ID NO: 98, a HCDR2 comprising SEQ ID NO: 99 and a HCDR3 comprising SEQ ID NO: 100; and the VL region amino acid sequence comprises a LCDR1 comprising SEQ ID NO: 96, a LCDR2 comprising SEQ ID NO: 101 and a LCDR3 comprising SEQ ID NO: 102; (r) the VH region amino acid sequence comprises a HCDR1 comprising SEQ ID NO: 103, a HCDR2 comprising SEQ ID NO: 104 and a HCDR3 comprising SEQ ID NO: 105; and the VL region amino acid sequence comprises a LCDR1 comprising SEQ ID NO: 96, a LCDR2 comprising SEQ ID NO: 106 and a LCDR3 comprising SEQ ID NO: 107; (s) the VH region amino acid sequence comprises a HCDR1 comprising SEQ ID NO: 108, a HCDR2 comprising SEQ ID NO: 94 and a HCDR3 comprising SEQ ID NO: 105; and the VL region amino acid sequence comprises a LCDR1 comprising SEQ ID NO: 109, a LCDR2 comprising SEQ ID NO: 85 and a LCDR3 comprising SEQ ID NO: 110; (t) the VH region amino acid sequence comprises a HCDR1 comprising SEQ ID NO: 111, a HCDR2 comprising SEQ ID NO: 94 and a HCDR3 comprising SEQ ID NO: 112; and the VL region amino acid sequence comprises a LCDR1 comprising SEQ ID NO: 96, a LCDR2 comprising SEQ ID NO: 85 and a LCDR3 comprising SEQ ID NO: 113; (u) the VH region amino acid sequence comprises a HCDR1 comprising SEQ ID NO: 87, a HCDR2 comprising SEQ ID NO: 94 and a HCDR3 comprising SEQ ID NO: 114; and the VL region amino acid sequence comprises a LCDR1 comprising SEQ ID NO: 115, a LCDR2 comprising SEQ ID NO: 85 and a LCDR3 comprising SEQ ID NO: 116; (v) the VH region amino acid sequence comprises a HCDR1 comprising SEQ ID NO: 93, a HCDR2 comprising SEQ ID NO: 90 and a HCDR3 comprising SEQ ID NO: 117; and the VL region amino acid sequence comprises a LCDR1 comprising SEQ ID NO: 118, a LCDR2 comprising SEQ ID NO: 85 and a LCDR3 comprising SEQ ID NO: 86; (w) the VH region amino acid sequence comprises a HCDR1 comprising SEQ ID NO: 119, a HCDR2 comprising SEQ ID NO: 120 and a HCDR3 comprising SEQ ID NO: 121; and the VL region amino acid sequence comprises a LCDR1 comprising SEQ ID NO: 57, a LCDR2 comprising SEQ ID NO: 58 and a LCDR3 comprising SEQ ID NO: 122; (x) the VH region amino acid sequence comprises a HCDR1 comprising SEQ ID NO: 123, a HCDR2 comprising SEQ ID NO: 124 and a HCDR3 comprising SEQ ID NO: 125; and the VL region amino acid sequence comprises a LCDR1 comprising SEQ ID NO: 126, a LCDR2 comprising SEQ ID NO: 127 and a LCDR3 comprising SEQ ID NO: 128; (y) the VH region amino acid sequence comprises a HCDR1 comprising SEQ ID NO: 123, a HCDR2 comprising SEQ ID NO: 124 and a HCDR3 comprising SEQ ID NO: 129; and the VL region amino acid sequence comprises a LCDR1 comprising SEQ ID NO: 57, a LCDR2 comprising SEQ ID NO: 58 and a LCDR3 comprising SEQ ID NO: 130; (z) the VH region amino acid sequence comprises a HCDR1 comprising SEQ ID NO: 131, a HCDR2 comprising SEQ ID NO: 132 and a HCDR3 comprising SEQ ID NO: 133; and the VL region amino acid sequence comprises a LCDR1 comprising SEQ ID NO: 126, a LCDR2 comprising SEQ ID NO: 134 and a LCDR3 comprising SEQ ID NO: 135; (aa) the VH region amino acid sequence comprises a HCDR1 comprising SEQ ID NO: 131, a HCDR2 comprising SEQ ID NO: 132 and a HCDR3 comprising SEQ ID NO: 136; and the VL region amino acid sequence comprises a LCDR1 comprising SEQ ID NO: 57, a LCDR2 comprising SEQ ID NO: 58 and a LCDR3 comprising SEQ ID NO: 137; (bb) the VH region amino acid sequence comprises a HCDR1 comprising SEQ ID NO: 54, a HCDR2 comprising SEQ ID NO: 138 and a HCDR3 comprising SEQ ID NO: 139; and the VL region amino acid sequence comprises a LCDR1 comprising SEQ ID NO: 57, a LCDR2 comprising SEQ ID NO: 58 and a LCDR3 comprising SEQ ID NO: 140; or (cc) the VH region amino acid sequence comprises a HCDR1 comprising SEQ ID NO: 81, a HCDR2 comprising SEQ ID NO: 82 and a HCDR3 comprising SEQ ID NO: 141; and the VL region amino acid sequence comprises a LCDR1 comprising SEQ ID NO: 142, a LCDR2 comprising SEQ ID NO: 143 and a LCDR3 comprising SEQ ID NO: 144.

Provided herein is an anti-VISTA antibody or an antigen-binding portion thereof, wherein the antibody or antigen-binding portion comprises a VH region and a VL region, wherein (a) the VH region amino acid sequence comprises SEQ ID NO: 1 and the VL region amino acid sequence comprises SEQ ID NO: 2; (b) the VH region amino acid sequence comprises SEQ ID NO: 3 and the VL region amino acid sequence comprises SEQ ID NO: 2; (c) the VH region amino acid sequence comprises SEQ ID NO: 4 and the VL region amino acid sequence comprises SEQ ID NO: 2; (d) the VH region amino acid sequence comprises SEQ ID NO: 5 and the VL region amino acid sequence comprises SEQ ID NO: 6; (e) the VH region amino acid sequence comprises SEQ ID NO: 7 and the VL region amino acid sequence comprises SEQ ID NO: 8; (f) the VH region amino acid sequence comprises SEQ ID NO: 9 and the VL region amino acid sequence comprises SEQ ID NO: 10; (g) the VH region amino acid sequence comprises SEQ ID NO: 11 and the VL region amino acid sequence comprises SEQ ID NO: 12; (h) the VH region amino acid sequence comprises SEQ ID NO: 13 and the VL region amino acid sequence comprises SEQ ID NO: 14; (i) the VH region amino acid sequence comprises SEQ ID NO: 15 and the VL region amino acid sequence comprises SEQ ID NO: 16; (j) the VH region amino acid sequence comprises SEQ ID NO: 17 and the VL region amino acid sequence comprises SEQ ID NO: 18; (k) the VH region amino acid sequence comprises SEQ ID NO: 19 and the VL region amino acid sequence comprises SEQ ID NO: 20; (1) the VH region amino acid sequence comprises SEQ ID NO: 21 and the VL region amino acid sequence comprises SEQ ID NO: 22; (m) the VH region amino acid sequence comprises SEQ ID NO: 23 and the VL region amino acid sequence comprises SEQ ID NO: 22; (n) the VH region amino acid sequence comprises SEQ ID NO: 24 and the VL region amino acid sequence comprises SEQ ID NO: 22; (o) the VH region amino acid sequence comprises SEQ ID NO: 25 and the VL region amino acid sequence comprises SEQ ID NO: 22; (p) the VH region amino acid sequence comprises SEQ ID NO: 26 and the VL region amino acid sequence comprises SEQ ID NO: 27; (q) the VH region amino acid sequence comprises SEQ ID NO: 28 and the VL region amino acid sequence comprises SEQ ID NO: 29; (r) the VH region amino acid sequence comprises SEQ ID NO: 30 and the VL region amino acid sequence comprises SEQ ID NO: 31; (s) the VH region amino acid sequence comprises SEQ ID NO: 32 and the VL region amino acid sequence comprises SEQ ID NO: 33; (t) the VH region amino acid sequence comprises SEQ ID NO: 34 and the VL region amino acid sequence comprises SEQ ID NO: 35; (u) the VH region amino acid sequence comprises SEQ ID NO: 36 and the VL region amino acid sequence comprises SEQ ID NO: 37; (v) the VH region amino acid sequence comprises SEQ ID NO: 38 and the VL region amino acid sequence comprises SEQ ID NO: 39; (w) the VH region amino acid sequence comprises SEQ ID NO: 40 and the VL region amino acid sequence comprises SEQ ID NO: 41; (x) the VH region amino acid sequence comprises SEQ ID NO: 42 and the VL region amino acid sequence comprises SEQ ID NO: 43; (y) the VH region amino acid sequence comprises SEQ ID NO: 44 and the VL region amino acid sequence comprises SEQ ID NO: 45; (z) the VH region amino acid sequence comprises SEQ ID NO: 46 and the VL region amino acid sequence comprises SEQ ID NO: 47; (aa) the VH region amino acid sequence comprises SEQ ID NO: 48 and the VL region amino acid sequence comprises SEQ ID NO: 49; (bb) the VH region amino acid sequence comprises SEQ ID NO: 50 and the VL region amino acid sequence comprises SEQ ID NO: 51; or (cc) the VH region amino acid sequence comprises SEQ ID NO: 52 and the VL region amino acid sequence comprises SEQ ID NO: 53.

Provided herein is an anti-VISTA antibody or an antigen-binding portion thereof, wherein the antibody or antigen-binding portion comprises a VH region and a VL region, wherein the VH region amino acid sequence comprises SEQ ID NO: 1, 3, 4, 5, 7, 9, 11, 13, 15, 17, 19, 21, 23, 24, 25, 26, 28, 30, 32, 34, 36, 38, 40, 42, 44, 46, 48, 50 or 52, or an amino acid sequence that is at least 95%, 96%, 97%, 98% or 99% identical to the amino acid sequence of SEQ ID NO: 1, 3, 4, 5, 7, 9, 11, 13, 15, 17, 19, 21, 23, 24, 25, 26, 28, 30, 32, 34, 36, 38, 40, 42, 44, 46, 48, 50 or 52.

Provided herein is an anti-VISTA antibody or an antigen-binding portion thereof, wherein the antibody or antigen-binding portion comprises a VH region and a VL region, wherein the VL region amino acid sequence comprises SEQ ID NO: 2, 6, 8, 10, 12, 14, 16, 18, 20, 22, 27, 29, 31, 33, 35, 37, 39, 41, 43, 45, 47, 49, 51 or 53, or an amino acid sequence that is at least 95%, 96%, 97%, 98% or 99% identical to the amino acid sequence of SEQ ID NO: 2, 6, 8, 10, 12, 14, 16, 18, 20, 22, 27, 29, 31, 33, 35, 37, 39, 41, 43, 45, 47, 49, 51 or 53.

Provided herein is an anti-VISTA antibody or an antigen-binding portion thereof, wherein the antibody or antigen-binding portion comprises a VH region and a VL region, wherein (a) the VH region amino acid sequence comprises SEQ ID NO: 1, or an amino acid sequence that is at least 95%, 96%, 97%, 98% or 99% identical to the amino acid sequence of SEQ ID NO: 1; and the VL region amino acid sequence comprises SEQ ID NO: 2, or an amino acid sequence that is at least 95%, 96%, 97%, 98% or 99% identical to the amino acid sequence of SEQ ID NO: 2; (b) the VH region amino acid sequence comprises SEQ ID NO: 3, or an amino acid sequence that is at least 95%, 96%, 97%, 98% or 99% identical to the amino acid sequence of SEQ ID NO: 3; and the VL region amino acid sequence comprises SEQ ID NO: 2, or an amino acid sequence that is at least 95%, 96%, 97%, 98% or 99% identical to the amino acid sequence of SEQ ID NO: 2; (c) the VH region amino acid sequence comprises SEQ ID NO: 4, or an amino acid sequence that is at least 95%, 96%, 97%, 98% or 99% identical to the amino acid sequence of SEQ ID NO: 4; and the VL region amino acid sequence comprises SEQ ID NO: 2, or an amino acid sequence that is at least 95%, 96%, 97%, 98% or 99% identical to the amino acid sequence of SEQ ID NO: 2; (d) the VH region amino acid sequence comprises SEQ ID NO: 5, or an amino acid sequence that is at least 95%, 96%, 97%, 98% or 99% identical to the amino acid sequence of SEQ ID NO: 5; and the VL region amino acid sequence comprises SEQ ID NO: 6, or an amino acid sequence that is at least 95%, 96%, 97%, 98% or 99% identical to the amino acid sequence of SEQ ID NO: 6; (e) the VH region amino acid sequence comprises SEQ ID NO: 7, or an amino acid sequence that is at least 95%, 96%, 97%, 98% or 99% identical to the amino acid sequence of SEQ ID NO: 7; and the VL region amino acid sequence comprises SEQ ID NO: 8, or an amino acid sequence that is at least 95%, 96%, 97%, 98% or 99% identical to the amino acid sequence of SEQ ID NO: 8; (f) the VH region amino acid sequence comprises SEQ ID NO: 9, or an amino acid sequence that is at least 95%, 96%, 97%, 98% or 99% identical to the amino acid sequence of SEQ ID NO: 9; and the VL region amino acid sequence comprises SEQ ID NO: 10, or an amino acid sequence that is at least 95%, 96%, 97%, 98% or 99% identical to the amino acid sequence of SEQ ID NO: 10; (g) the VH region amino acid sequence comprises SEQ ID NO: 11, or an amino acid sequence that is at least 95%, 96%, 97%, 98% or 99% identical to the amino acid sequence of SEQ ID NO: 11; and the VL region amino acid sequence comprises SEQ ID NO: 12, or an amino acid sequence that is at least 95%, 96%, 97%, 98% or 99% identical to the amino acid sequence of SEQ ID NO: 12; (h) the VH region amino acid sequence comprises SEQ ID NO: 13, or an amino acid sequence that is at least 95%, 96%, 97%, 98% or 99% identical to the amino acid sequence of SEQ ID NO: 13; and the VL region amino acid sequence comprises SEQ ID NO: 14, or an amino acid sequence that is at least 95%, 96%, 97%, 98% or 99% identical to the amino acid sequence of SEQ ID NO: 14; (i) the VH region amino acid sequence comprises SEQ ID NO: 15, or an amino acid sequence that is at least 95%, 96%, 97%, 98% or 99% identical to the amino acid sequence of SEQ ID NO: 15; and the VL region amino acid sequence comprises SEQ ID NO: 16, or an amino acid sequence that is at least 95%, 96%, 97%, 98% or 99% identical to the amino acid sequence of SEQ ID NO: 16; (j) the VH region amino acid sequence comprises SEQ ID NO: 17, or an amino acid sequence that is at least 95%, 96%, 97%, 98% or 99% identical to the amino acid sequence of SEQ ID NO: 17; and the VL region amino acid sequence comprises SEQ ID NO: 18, or an amino acid sequence that is at least 95%, 96%, 97%, 98% or 99% identical to the amino acid sequence of SEQ ID NO: 18; (k) the VH region amino acid sequence comprises SEQ ID NO: 19, or an amino acid sequence that is at least 95%, 96%, 97%, 98% or 99% identical to the amino acid sequence of SEQ ID NO: 19; and the VL region amino acid sequence comprises SEQ ID NO: 20, or an amino acid sequence that is at least 95%, 96%, 97%, 98% or 99% identical to the amino acid sequence of SEQ ID NO: 20; (1) the VH region amino acid sequence comprises SEQ ID NO: 21, or an amino acid sequence that is at least 95%, 96%, 97%, 98% or 99% identical to the amino acid sequence of SEQ ID NO: 21; and the VL region amino acid sequence comprises SEQ ID NO: 22, or an amino acid sequence that is at least 95%, 96%, 97%, 98% or 99% identical to the amino acid sequence of SEQ ID NO: 22; (m) the VH region amino acid sequence comprises SEQ ID NO: 23, or an amino acid sequence that is at least 95%, 96%, 97%, 98% or 99% identical to the amino acid sequence of SEQ ID NO: 23; and the VL region amino acid sequence comprises SEQ ID NO: 22, or an amino acid sequence that is at least 95%, 96%, 97%, 98% or 99% identical to the amino acid sequence of SEQ ID NO: 22; (n) the VH region amino acid sequence comprises SEQ ID NO: 24, or an amino acid sequence that is at least 95%, 96%, 97%, 98% or 99% identical to the amino acid sequence of SEQ ID NO: 24; and the VL region amino acid sequence comprises SEQ ID NO: 22, or an amino acid sequence that is at least 95%, 96%, 97%, 98% or 99% identical to the amino acid sequence of SEQ ID NO: 22; (o) the VH region amino acid sequence comprises SEQ ID NO: 25, or an amino acid sequence that is at least 95%, 96%, 97%, 98% or 99% identical to the amino acid sequence of SEQ ID NO: 25; and the VL region amino acid sequence comprises SEQ ID NO: 22, or an amino acid sequence that is at least 95%, 96%, 97%, 98% or 99% identical to the amino acid sequence of SEQ ID NO: 22; (p) the VH region amino acid sequence comprises SEQ ID NO: 26, or an amino acid sequence that is at least 95%, 96%, 97%, 98% or 99% identical to the amino acid sequence of SEQ ID NO: 26; and the VL region amino acid sequence comprises SEQ ID NO: 27, or an amino acid sequence that is at least 95%, 96%, 97%, 98% or 99% identical to the amino acid sequence of SEQ ID NO: 27; (q) the VH region amino acid sequence comprises SEQ ID NO: 28, or an amino acid sequence that is at least 95%, 96%, 97%, 98% or 99% identical to the amino acid sequence of SEQ ID NO: 28; and the VL region amino acid sequence comprises SEQ ID NO: 29, or an amino acid sequence that is at least 95%, 96%, 97%, 98% or 99% identical to the amino acid sequence of SEQ ID NO: 29; (r) the VH region amino acid sequence comprises SEQ ID NO: 30, or an amino acid sequence that is at least 95%, 96%, 97%, 98% or 99% identical to the amino acid sequence of SEQ ID NO: 30; and the VL region amino acid sequence comprises SEQ ID NO: 31, or an amino acid sequence that is at least 95%, 96%, 97%, 98% or 99% identical to the amino acid sequence of SEQ ID NO: 31; (s) the VH region amino acid sequence comprises SEQ ID NO: 32, or an amino acid sequence that is at least 95%, 96%, 97%, 98% or 99% identical to the amino acid sequence of SEQ ID NO: 32; and the VL region amino acid sequence comprises SEQ ID NO: 33, or an amino acid sequence that is at least 95%, 96%, 97%, 98% or 99% identical to the amino acid sequence of SEQ ID NO: 33; (t) the VH region amino acid sequence comprises SEQ ID NO: 34, or an amino acid sequence that is at least 95%, 96%, 97%, 98% or 99% identical to the amino acid sequence of SEQ ID NO: 34; and the VL region amino acid sequence comprises SEQ ID NO: 35, or an amino acid sequence that is at least 95%, 96%, 97%, 98% or 99% identical to the amino acid sequence of SEQ ID NO: 35; (u) the VH region amino acid sequence comprises SEQ ID NO: 36, or an amino acid sequence that is at least 95%, 96%, 97%, 98% or 99% identical to the amino acid sequence of SEQ ID NO: 36; and the VL region amino acid sequence comprises SEQ ID NO: 37, or an amino acid sequence that is at least 95%, 96%, 97%, 98% or 99% identical to the amino acid sequence of SEQ ID NO: 37; (v) the VH region amino acid sequence comprises SEQ ID NO: 38, or an amino acid sequence that is at least 95%, 96%, 97%, 98% or 99% identical to the amino acid sequence of SEQ ID NO: 38; and the VL region amino acid sequence comprises SEQ ID NO: 39, or an amino acid sequence that is at least 95%, 96%, 97%, 98% or 99% identical to the amino acid sequence of SEQ ID NO: 39; (w) the VH region amino acid sequence comprises SEQ ID NO: 40, or an amino acid sequence that is at least 95%, 96%, 97%, 98% or 99% identical to the amino acid sequence of SEQ ID NO: 40; and the VL region amino acid sequence comprises SEQ ID NO: 41, or an amino acid sequence that is at least 95%, 96%, 97%, 98% or 99% identical to the amino acid sequence of SEQ ID NO: 41; (x) the VH region amino acid sequence comprises SEQ ID NO: 42, or an amino acid sequence that is at least 95%, 96%, 97%, 98% or 99% identical to the amino acid sequence of SEQ ID NO: 42; and the VL region amino acid sequence comprises SEQ ID NO: 43, or an amino acid sequence that is at least 95%, 96%, 97%, 98% or 99% identical to the amino acid sequence of SEQ ID NO: 43; (y) the VH region amino acid sequence comprises SEQ ID NO: 44, or an amino acid sequence that is at least 95%, 96%, 97%, 98% or 99% identical to the amino acid sequence of SEQ ID NO: 44; and the VL region amino acid sequence comprises SEQ ID NO: 45, or an amino acid sequence that is at least 95%, 96%, 97%, 98% or 99% identical to the amino acid sequence of SEQ ID NO: 45; (z) the VH region amino acid sequence comprises SEQ ID NO: 46, or an amino acid sequence that is at least 95%, 96%, 97%, 98% or 99% identical to the amino acid sequence of SEQ ID NO: 46; and the VL region amino acid sequence comprises SEQ ID NO: 47, or an amino acid sequence that is at least 95%, 96%, 97%, 98% or 99% identical to the amino acid sequence of SEQ ID NO: 47; (aa) the VH region amino acid sequence comprises SEQ ID NO: 48, or an amino acid sequence that is at least 95%, 96%, 97%, 98% or 99% identical to the amino acid sequence of SEQ ID NO: 48; and the VL region amino acid sequence comprises SEQ ID NO: 49, or an amino acid sequence that is at least 95%, 96%, 97%, 98% or 99% identical to the amino acid sequence of SEQ ID NO: 49; (bb) the VH region amino acid sequence comprises SEQ ID NO: 50, or an amino acid sequence that is at least 95%, 96%, 97%, 98% or 99% identical to the amino acid sequence of SEQ ID NO: 50; and the VL region amino acid sequence comprises SEQ ID NO: 51, or an amino acid sequence that is at least 95%, 96%, 97%, 98% or 99% identical to the amino acid sequence of SEQ ID NO: 51; or (cc) the VH region amino acid sequence comprises SEQ ID NO: 52, or an amino acid sequence that is at least 95%, 96%, 97%, 98% or 99% identical to the amino acid sequence of SEQ ID NO: 52; and the VL region amino acid sequence comprises SEQ ID NO: 53, or an amino acid sequence that is at least 95%, 96%, 97%, 98% or 99% identical to the amino acid sequence of SEQ ID NO: 53.

Provided herein is an anti-VISTA antibody or an antigen-binding portion thereof, wherein the antibody or antigen-binding portion comprises a VH region and a VL region, wherein the VH and VL region sequences are those of antibody 55873, 67373, 67374, 67375, 67376, 67377, 67378, 67379, 67380, 67381, 67382, 55902, 67414, 67415, 67416, 67417, 67418, 67419, 67420, 67422, 67423, 67424, 55872, 55879, 55880, 55899, 55901, 55932 or 55934, with 1, 2 or 3 conservative amino acid substitutions in the VH region sequence, the VL region sequence, or both the VH region and the VL region sequences. In some embodiments, conservative amino acid substitutions are made only in the FR sequences and not in the CDR sequences of an antibody or antigen-binding portion.

In some embodiments, an anti-VISTA antibody or antigen-binding portion provided herein is monoclonal. The term “monoclonal antibody” (Mab) refers to an antibody, or antigen-binding portion thereof, that is derived from a single copy or clone, including for example any eukaryotic, prokaryotic, or phage clone, and not the method by which it is produced. Preferably, a monoclonal antibody exists in a homogeneous or substantially homogeneous population.

In some embodiments, the antibody or antigen-binding portion provided herein may be isolated.

In some embodiments, an anti-VISTA antibody or antigen-binding portion provided herein is human (e.g., fully human). A human antibody comprises human CDR sequences, human framework sequences and human constant region sequences.

In some embodiments, an anti-VISTA antibody or antigen-binding portion provided herein is chimeric. The term “chimeric” is intended to refer to an antibody molecule, or an antigen-binding portion thereof, in which the variable domain sequences are derived from one species and at least one constant region sequence is derived from another species. Examples of chimeric antibodies and suitable techniques for their generation are provided in U.S. Pat. Nos. 4,816,567; 4,975,369; and 4,816,397, each of which is incorporated herein by reference in its entirety.

The present disclosure encompasses modifications to the amino acid sequence of the antibody molecule or antigen-binding portion thereof as defined herein. For example, the disclosure includes antibody molecules and corresponding antigen-binding portions thereof comprising functionally equivalent variable regions and CDRs which do not significantly affect their properties as well as variants which have enhanced or decreased activity and/or affinity. For example, the amino acid sequence may be mutated to obtain an antibody with the desired binding affinity to VISTA. Insertions which include amino- and/or carboxyl-terminal fusions ranging in length from one residue to polypeptides containing a hundred or more residues, as well as intrasequence insertions of single or multiple amino acid residues, are envisaged. Examples of terminal insertions include an antibody molecule with an N-terminal methionyl residue or the antibody molecule fused to an epitope tag. Other insertional variants of the antibody molecule include the fusion to the N- or C-terminus of the antibody of an enzyme or a polypeptide which increases the half-life of the antibody in the blood circulation.

In some embodiments, the anti-VISTA antibody or antigen-binding portion provided herein may include glycosylated and non-glycosylated polypeptides, as well as polypeptides with other post-translational modifications, such as, for example, glycosylation with different sugars, acetylation, and phosphorylation. The antibody or antigen-binding portion may be mutated to alter such post-translational modifications, for example by adding, removing or replacing one or more amino acid residues to form or remove a glycosylation site.

In some embodiments, the anti-VISTA antibody or antigen-binding portion provided herein may be modified for example by amino acid substitution to remove potential proteolytic sites in the antibody or portion.

In some embodiments, an anti-VISTA antibody or an antigen-binding portion thereof comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region comprises one or more mutations to increase FcγR binding, antibody-dependent cell-mediated cytotoxicity activity, and/or complement-dependent cytotoxicity activity. In some embodiments, the immunoglobulin constant region is immunologically inert. In some embodiments, the immunoglobulin constant region comprises one or more mutations to reduce or prevent FcγR binding, antibody-dependent cell-mediated cytotoxicity activity, and/or complement-dependent cytotoxicity activity. In some embodiments, the immunoglobulin constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU index as in Kabat. In some embodiments, a position of an amino acid residue in a constant region of an immunoglobulin molecule is numbered according to the EU index as in Kabat (Ward et al., 1995 Therap. Immunol. 2:77-94).

In some embodiments, an anti-VISTA antibody or an antigen-binding portion thereof may comprise an immunoglobulin light chain constant region that is a kappa light chain constant region or a lambda light chain constant region.

In some embodiments, an anti-VISTA antibody may comprise an immunoglobulin constant region comprising any one of the amino acid sequences in Table 30. The Fc region sequences in Table 30 begin at the CH1 domain. In some embodiments, an anti-VISTA antibody may comprise an immunoglobulin constant region comprising an amino acid sequence of an Fc region of human IgG4 or human IgG1. In some embodiments, an anti-VISTA antibody may comprise an immunoglobulin constant region comprising an amino acid sequence of an Fc region of human IgG4, human IgG4(S228P), human IgG2, human IgG1, human IgG1 effector null. For example, the human IgG4(S228P) Fc region comprises the following substitution compared to the wild-type human IgG4 Fc region: S228P. For example, the human IgG1 effector null Fc region comprises the following substitutions compared to the wild-type human IgG1 Fc region: L234A, L235A and G237A. In some embodiments, an anti-VISTA antibody may comprise an immunoglobulin constant region comprising any one of SEQ ID NOS: 145-149. In some embodiments, an anti-VISTA antibody may comprise the six CDR amino acid sequences of any one of the clones in Tables 1-29 and any one of the Fc region amino acid sequences in Table 30. In some embodiments, an anti-VISTA antibody may comprise an immunoglobulin heavy chain constant region comprising any one of the Fc region amino acid sequences in Table 30 and an immunoglobulin light chain constant region that is a kappa light chain constant region or a lambda light chain constant region.

Further provided herein is an immunoconjugate comprising an anti-VISTA antibody or an antigen-binding portion thereof, linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic peptide, a therapeutic nucleic acid, an anti-angiogenic agent, an anti-proliferative agent, or a pro-apoptotic agent.

Examples of suitable therapeutic agents include, but are not limited to, immunomodulatory agents, cytotoxins, radioisotopes, chemotherapeutic agents, anti-angiogenic agents, antiproliferative agents, pro-apoptotic agents, and cytostatic and cytolytic enzymes (for example RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include, but are not limited to, JAK kinase inhibitors, taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers may also be used in immunoconjugates. In addition, conjugates can be made using secondary carriers as the cytotoxic agent, such as liposomes or polymers, Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

### Pharmaceutical Compositions

The anti-VISTA antibodies and antigen-binding portions provided herein (also referred to herein as “active compounds”) can be incorporated into pharmaceutical compositions suitable for administration. Such compositions typically comprise an anti-VISTA antibody or antigen-binding portion (or an immunoconjugate comprising said antibody or portion), and a pharmaceutically acceptable carrier, diluent or excipient. Such materials should be non-toxic and should not interfere with the efficacy of the anti-VISTA antibody or antigen-binding fragment thereof. The precise nature of the carrier or other material will depend on the route of administration, which may be by injection, bolus, infusion, or any other suitable route, as discussed below.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the anti-VISTA antibody or antigen-binding portion thereof, an increase in its lifespan and/or in its efficacy in the body or an increase in its solubility in solution.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the anti-VISTA antibody or antigen-binding portion thereof may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Antibodies

Provided herein is a nucleic acid molecule (e.g., an isolated nucleic acid molecule) encoding an amino acid sequence of an anti-VISTA antibody or anti-VISTA antigen-binding portion described herein (or an amino acid sequence of a (i) VH region, (ii) a VL region, or (iii) both a VH region and a VL region of an antibody or antigen-binding portion). Further provided herein is a nucleic acid molecule (e.g., an isolated nucleic acid molecule) encoding (i) a heavy chain, (ii) a light chain, or (iii) both a heavy chain and a light chain of an anti-VISTA antibody or anti-VISTA antigen-binding portion described herein. In some embodiments, a nucleic acid molecule encoding a VH region, a VL region, a heavy chain or a light chain comprises a signal sequence. In some embodiments, a nucleic acid molecule encoding a VH region, a VL region, a heavy chain or a light chain does not comprise a signal sequence

In some embodiments, a nucleic acid molecule encodes an amino acid sequence of a VH region and a VL region of an anti-VISTA antibody or an antigen-binding portion thereof, wherein: (a) the VH region amino acid sequence comprises a HCDR1 comprising SEQ ID NO: 54, a HCDR2 comprising SEQ ID NO: 55 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (b) the VH region amino acid sequence comprises a HCDR1 comprising SEQ ID NO: 60, a HCDR2 comprising SEQ ID NO: 61 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (c) the VH region amino acid sequence comprises a HCDR1 comprising SEQ ID NO: 62, a HCDR2 comprising SEQ ID NO: 63 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59; (d) the VH region amino acid sequence comprises a HCDR1 comprising SEQ ID NO: 64, a HCDR2 comprising SEQ ID NO: 65 and a HCDR3 comprising SEQ ID NO: 66; and the VL region amino acid sequence comprises a LCDR1 comprising SEQ ID NO: 57, a LCDR2 comprising SEQ ID NO: 67 and a LCDR3 comprising SEQ ID NO: 68; (e) the VH region amino acid sequence comprises a HCDR1 comprising SEQ ID NO: 69, a HCDR2 comprising SEQ ID NO: 70 and a HCDR3 comprising SEQ ID NO: 56; and the VL region amino acid sequence comprises a LCDR1 comprising SEQ ID NO: 57, a LCDR2 comprising SEQ ID NO: 67 and a LCDR3 comprising SEQ ID NO: 71; (f) the VH region amino acid sequence comprises a HCDR1 comprising SEQ ID NO: 60, a HCDR2 comprising SEQ ID NO: 61 and a HCDR3 comprising SEQ ID NO: 72; and the VL region amino acid sequence comprises a LCDR1 comprising SEQ ID NO: 57, a LCDR2 comprising SEQ ID NO: 58 and a LCDR3 comprising SEQ ID NO: 68; (g) the VH region amino acid sequence comprises a HCDR1 comprising SEQ ID NO: 73, a HCDR2 comprising SEQ ID NO: 65 and a HCDR3 comprising SEQ ID NO: 74; and the VL region amino acid sequence comprises a LCDR1 comprising SEQ ID NO: 57, a LCDR2 comprising SEQ ID NO: 58 and a LCDR3 comprising SEQ ID NO: 71; (h) the VH region amino acid sequence comprises a HCDR1 comprising SEQ ID NO: 64, a HCDR2 comprising SEQ ID NO: 75 and a HCDR3 comprising SEQ ID NO: 66; and the VL region amino acid sequence comprises a LCDR1 comprising SEQ ID NO: 57, a LCDR2 comprising SEQ ID NO: 76 and a LCDR3 comprising SEQ ID NO: 68; (i) the VH region amino acid sequence comprises a HCDR1 comprising SEQ ID NO: 64, a HCDR2 comprising SEQ ID NO: 65 and a HCDR3 comprising SEQ ID NO: 77; and the VL region amino acid sequence comprises a LCDR1 comprising SEQ ID NO: 57, a LCDR2 comprising SEQ ID NO: 78 and a LCDR3 comprising SEQ ID NO: 68; (j) the VH region amino acid sequence comprises a HCDR1 comprising SEQ ID NO: 60, a HCDR2 comprising SEQ ID NO: 75 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 68; (k) the VH region amino acid sequence comprises a HCDR1 comprising SEQ ID NO: 54, a HCDR2 comprising SEQ ID NO: 65 and a HCDR3 comprising SEQ ID NO: 79; and the VL region amino acid sequence comprises a LCDR1 comprising SEQ ID NO: 80, a LCDR2 comprising SEQ ID NO: 58 and a LCDR3 comprising SEQ ID NO: 68; (1) the VH region amino acid sequence comprises a HCDR1 comprising SEQ ID NO: 81, a HCDR2 comprising SEQ ID NO: 82 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (m) the VH region amino acid sequence comprises a HCDR1 comprising SEQ ID NO: 87, a HCDR2 comprising SEQ ID NO: 88 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (n) the VH region amino acid sequence comprises a HCDR1 comprising SEQ ID NO: 89, a HCDR2 comprising SEQ ID NO: 90 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (o) the VH region amino acid sequence comprises a HCDR1 comprising SEQ ID NO: 91, a HCDR2 comprising SEQ ID NO: 92 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86; (p) the VH region amino acid sequence comprises a HCDR1 comprising SEQ ID NO: 93, a HCDR2 comprising SEQ ID NO: 94 and a HCDR3 comprising SEQ ID NO: 95; and the VL region amino acid sequence comprises a LCDR1 comprising SEQ ID NO: 96, a LCDR2 comprising SEQ ID NO: 85 and a LCDR3 comprising SEQ ID NO: 97; (q) the VH region amino acid sequence comprises a HCDR1 comprising SEQ ID NO: 98, a HCDR2 comprising SEQ ID NO: 99 and a HCDR3 comprising SEQ ID NO: 100; and the VL region amino acid sequence comprises a LCDR1 comprising SEQ ID NO: 96, a LCDR2 comprising SEQ ID NO: 101 and a LCDR3 comprising SEQ ID NO: 102; (r) the VH region amino acid sequence comprises a HCDR1 comprising SEQ ID NO: 103, a HCDR2 comprising SEQ ID NO: 104 and a HCDR3 comprising SEQ ID NO: 105; and the VL region amino acid sequence comprises a LCDR1 comprising SEQ ID NO: 96, a LCDR2 comprising SEQ ID NO: 106 and a LCDR3 comprising SEQ ID NO: 107; (s) the VH region amino acid sequence comprises a HCDR1 comprising SEQ ID NO: 108, a HCDR2 comprising SEQ ID NO: 94 and a HCDR3 comprising SEQ ID NO: 105; and the VL region amino acid sequence comprises a LCDR1 comprising SEQ ID NO: 109, a LCDR2 comprising SEQ ID NO: 85 and a LCDR3 comprising SEQ ID NO: 110; (t) the VH region amino acid sequence comprises a HCDR1 comprising SEQ ID NO: 111, a HCDR2 comprising SEQ ID NO: 94 and a HCDR3 comprising SEQ ID NO: 112; and the VL region amino acid sequence comprises a LCDR1 comprising SEQ ID NO: 96, a LCDR2 comprising SEQ ID NO: 85 and a LCDR3 comprising SEQ ID NO: 113; (u) the VH region amino acid sequence comprises a HCDR1 comprising SEQ ID NO: 87, a HCDR2 comprising SEQ ID NO: 94 and a HCDR3 comprising SEQ ID NO: 114; and the VL region amino acid sequence comprises a LCDR1 comprising SEQ ID NO: 115, a LCDR2 comprising SEQ ID NO: 85 and a LCDR3 comprising SEQ ID NO: 116; (v) the VH region amino acid sequence comprises a HCDR1 comprising SEQ ID NO: 93, a HCDR2 comprising SEQ ID NO: 90 and a HCDR3 comprising SEQ ID NO: 117; and the VL region amino acid sequence comprises a LCDR1 comprising SEQ ID NO: 118, a LCDR2 comprising SEQ ID NO: 85 and a LCDR3 comprising SEQ ID NO: 86; (w) the VH region amino acid sequence comprises a HCDR1 comprising SEQ ID NO: 119, a HCDR2 comprising SEQ ID NO: 120 and a HCDR3 comprising SEQ ID NO: 121; and the VL region amino acid sequence comprises a LCDR1 comprising SEQ ID NO: 57, a LCDR2 comprising SEQ ID NO: 58 and a LCDR3 comprising SEQ ID NO: 122; (x) the VH region amino acid sequence comprises a HCDR1 comprising SEQ ID NO: 123, a HCDR2 comprising SEQ ID NO: 124 and a HCDR3 comprising SEQ ID NO: 125; and the VL region amino acid sequence comprises a LCDR1 comprising SEQ ID NO: 126, a LCDR2 comprising SEQ ID NO: 127 and a LCDR3 comprising SEQ ID NO: 128; (y) the VH region amino acid sequence comprises a HCDR1 comprising SEQ ID NO: 123, a HCDR2 comprising SEQ ID NO: 124 and a HCDR3 comprising SEQ ID NO: 129; and the VL region amino acid sequence comprises a LCDR1 comprising SEQ ID NO: 57, a LCDR2 comprising SEQ ID NO: 58 and a LCDR3 comprising SEQ ID NO: 130; (z) the VH region amino acid sequence comprises a HCDR1 comprising SEQ ID NO: 131, a HCDR2 comprising SEQ ID NO: 132 and a HCDR3 comprising SEQ ID NO: 133; and the VL region amino acid sequence comprises a LCDR1 comprising SEQ ID NO: 126, a LCDR2 comprising SEQ ID NO: 134 and a LCDR3 comprising SEQ ID NO: 135; (aa) the VH region amino acid sequence comprises a HCDR1 comprising SEQ ID NO: 131, a HCDR2 comprising SEQ ID NO: 132 and a HCDR3 comprising SEQ ID NO: 136; and the VL region amino acid sequence comprises a LCDR1 comprising SEQ ID NO: 57, a LCDR2 comprising SEQ ID NO: 58 and a LCDR3 comprising SEQ ID NO: 137; (bb) the VH region amino acid sequence comprises a HCDR1 comprising SEQ ID NO: 54, a HCDR2 comprising SEQ ID NO: 138 and a HCDR3 comprising SEQ ID NO: 139; and the VL region amino acid sequence comprises a LCDR1 comprising SEQ ID NO: 57, a LCDR2 comprising SEQ ID NO: 58 and a LCDR3 comprising SEQ ID NO: 140; or (cc) the VH region amino acid sequence comprises a HCDR1 comprising SEQ ID NO: 81, a HCDR2 comprising SEQ ID NO: 82 and a HCDR3 comprising SEQ ID NO: 141; and the VL region amino acid sequence comprises a LCDR1 comprising SEQ ID NO: 142, a LCDR2 comprising SEQ ID NO: 143 and a LCDR3 comprising SEQ ID NO: 144.

In some embodiments, a nucleic acid molecule encodes an amino acid sequence of a VH region and a VL region of an anti-VISTA antibody or an antigen-binding portion thereof, wherein (a) the VH region amino acid sequence comprises SEQ ID NO: 1 and the VL region amino acid sequence comprises SEQ ID NO: 2; (b) the VH region amino acid sequence comprises SEQ ID NO: 3 and the VL region amino acid sequence comprises SEQ ID NO: 2; (c) the VH region amino acid sequence comprises SEQ ID NO: 4 and the VL region amino acid sequence comprises SEQ ID NO: 2; (d) the VH region amino acid sequence comprises SEQ ID NO: 5 and the VL region amino acid sequence comprises SEQ ID NO: 6; (e) the VH region amino acid sequence comprises SEQ ID NO: 7 and the VL region amino acid sequence comprises SEQ ID NO: 8; (f) the VH region amino acid sequence comprises SEQ ID NO: 9 and the VL region amino acid sequence comprises SEQ ID NO: 10; (g) the VH region amino acid sequence comprises SEQ ID NO: 11 and the VL region amino acid sequence comprises SEQ ID NO: 12; (h) the VH region amino acid sequence comprises SEQ ID NO: 13 and the VL region amino acid sequence comprises SEQ ID NO: 14; (i) the VH region amino acid sequence comprises SEQ ID NO: 15 and the VL region amino acid sequence comprises SEQ ID NO: 16; (j) the VH region amino acid sequence comprises SEQ ID NO: 17 and the VL region amino acid sequence comprises SEQ ID NO: 18; (k) the VH region amino acid sequence comprises SEQ ID NO: 19 and the VL region amino acid sequence comprises SEQ ID NO: 20; (1) the VH region amino acid sequence comprises SEQ ID NO: 21 and the VL region amino acid sequence comprises SEQ ID NO: 22; (m) the VH region amino acid sequence comprises SEQ ID NO: 23 and the VL region amino acid sequence comprises SEQ ID NO: 22; (n) the VH region amino acid sequence comprises SEQ ID NO: 24 and the VL region amino acid sequence comprises SEQ ID NO: 22; (o) the VH region amino acid sequence comprises SEQ ID NO: 25 and the VL region amino acid sequence comprises SEQ ID NO: 22; (p) the VH region amino acid sequence comprises SEQ ID NO: 26 and the VL region amino acid sequence comprises SEQ ID NO: 27; (q) the VH region amino acid sequence comprises SEQ ID NO: 28 and the VL region amino acid sequence comprises SEQ ID NO: 29; (r) the VH region amino acid sequence comprises SEQ ID NO: 30 and the VL region amino acid sequence comprises SEQ ID NO: 31; (s) the VH region amino acid sequence comprises SEQ ID NO: 32 and the VL region amino acid sequence comprises SEQ ID NO: 33; (t) the VH region amino acid sequence comprises SEQ ID NO: 34 and the VL region amino acid sequence comprises SEQ ID NO: 35; (u) the VH region amino acid sequence comprises SEQ ID NO: 36 and the VL region amino acid sequence comprises SEQ ID NO: 37; (v) the VH region amino acid sequence comprises SEQ ID NO: 38 and the VL region amino acid sequence comprises SEQ ID NO: 39; (w) the VH region amino acid sequence comprises SEQ ID NO: 40 and the VL region amino acid sequence comprises SEQ ID NO: 41; (x) the VH region amino acid sequence comprises SEQ ID NO: 42 and the VL region amino acid sequence comprises SEQ ID NO: 43; (y) the VH region amino acid sequence comprises SEQ ID NO: 44 and the VL region amino acid sequence comprises SEQ ID NO: 45; (z) the VH region amino acid sequence comprises SEQ ID NO: 46 and the VL region amino acid sequence comprises SEQ ID NO: 47; (aa) the VH region amino acid sequence comprises SEQ ID NO: 48 and the VL region amino acid sequence comprises SEQ ID NO: 49; (bb) the VH region amino acid sequence comprises SEQ ID NO: 50 and the VL region amino acid sequence comprises SEQ ID NO: 51; or (cc) the VH region amino acid sequence comprises SEQ ID NO: 52 and the VL region amino acid sequence comprises SEQ ID NO: 53.

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding both a VH region and a VL region of an anti-VISTA antibody or an antigen-binding portion thereof. In some embodiments, a recombinant host cell comprises (i) a first vector or a first nucleic acid molecule encoding a VH region of an anti-VISTA antibody or an antigen-binding portion thereof and (ii) a second vector or a second nucleic acid molecule encoding a VL region of an anti-VISTA antibody or an antigen-binding portion thereof.

Antibody molecules of the invention, or antigen-binding portion thereof, can be produced using techniques well known in the art, for example, recombinant technologies, phage display technologies, synthetic technologies, computational technologies or combinations of such technologies or other technologies readily known in the art.

Further provided herein is a method for producing an anti-VISTA antibody or an antigen-binding portion thereof, the method comprising: culturing a recombinant host cell comprising an expression vector described herein under conditions whereby the nucleic acid segment is expressed, thereby producing the anti-VISTA antibody or antigen-binding portion. The antibody or antigen-binding portion may then be isolated from the host cell or culture. Anti-VISTA antibodies and antigen-binding portions thereof can be produced by any of a variety of methods known to those skilled in the art. In certain embodiments, anti-VISTA antibodies and antigen-binding portions thereof can be produced recombinantly. For example, nucleic acid sequences encoding one or more of SEQ ID NOS: 1-144, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, antibody light chain proteins and heavy chain proteins are produced in a cell with a signal sequence that is removed upon production of a mature anti-VISTA antibody or antigen-binding portion thereof.

Those skilled in the art will be able to determine whether an antibody or antigen-binding portion comprising a given polypeptide sequence binds to VISTA protein without undue experimentation using standard methodologies, for example, Western blots, ELISA, and the like.

### Uses of Antibodies

Provided herein are methods and uses of the anti-VISTA antibodies, anti-VISTA antigen-binding portions, immunoconjugates and pharmaceutical compositions described herein for providing a therapeutic benefit to a subject with cancer.

An anti-VISTA antibody or antigen-binding portion thereof as described herein may be used in a method of treatment of the human or animal body, including prophylactic or preventative treatment (e.g., treatment before the onset of a condition in a subject to reduce the risk of the condition occurring in the subject; delay its onset; or reduce its severity after onset). The method of treatment may comprise administering the anti-VISTA antibody or antigen-binding portion to a subject in need thereof.

In some embodiments, an anti-VISTA antibody or antigen-binding portion thereof as described herein does not induce CRS when administered to a subject. In some embodiments, an anti-VISTA antibody or antigen-binding portion thereof as described herein induces minimal CRS when administered to a subject. In some embodiments, an anti-VISTA antibody or antigen-binding portion thereof as described herein induces minimal expression of any combination of IL-6, IL-10, CCL-2, CCL-5, CXCL-8, CXCL-10, IFN-γ, TNF-α, and IL-1RA, when administered to a subject.

Provided herein is a method for inducing an immune response in a subject, the method comprising administering to the subject a therapeutically effective amount of an anti-VISTA antibody or an antigen-binding portion thereof, an immunoconjugate or a pharmaceutical composition disclosed herein. In some embodiments, the immune response is an immune response against cancer cells. In some embodiments, the immune response is antibody-dependent cellular cytotoxicity, complement-dependent cytotoxicity or antibody-dependent cellular phagocytosis. In some embodiments, the immune response is an antibody response. In some embodiments, the immune response is a cytotoxic response.

Provided herein is a method for treating or preventing a cancer in a subject, the method comprising administering to the subject a therapeutically effective amount of an anti-VISTA antibody or an antigen-binding portion thereof, an immunoconjugate or a pharmaceutical composition disclosed herein. In some embodiments, the cancer is a hematologic malignancy. In some embodiments, the cancer is a myeloid malignancy. In some embodiments, the cancer is acute myeloid leukemia (AML). In some embodiments, the cancer is an epithelial cancer. In some embodiments, the cancer is a solid tumor. In some embodiments, the cancer is squamous cell carcinoma of head and neck, melanoma, non-small cell lung cancer or colon cancer.

In some embodiments, a subject overexpresses VISTA. In some embodiments, a subject overexpresses VISTA on blood cells. In some embodiments, a subject overexpresses VISTA on immune cells. In some embodiments, the immune cells are tumor-infiltrating immune cells. In some embodiments, the immune cells are myeloid cells or monocytes.

In some embodiments, a subject may be treated with an anti-VISTA antibody or an anti-VISTA antigen-binding portion, an immunoconjugate or a pharmaceutical composition described herein and an additional therapeutic agent or therapy that is used to treat a VISTA-mediated disease or disorder or a symptom or complication of a VISTA-mediated disease or disorder. The anti-VISTA antibody or an anti-VISTA antigen-binding portion and the additional therapeutic agent or therapy may be administered simultaneously or sequentially. In some embodiments, the additional therapeutic agent is an inhibitor of PD-1 or PD-L1.

In some embodiments, a subject does not respond or has developed a resistance to cancer treatment with an inhibitor of PD-1 or PD-L1. In some embodiments, a method provided herein further comprises administering to the subject an inhibitor of PD-1 or PD-L1. In some embodiments, administering an anti-VISTA antibody, anti-VISTA antigen-binding portion, immunoconjugate or pharmaceutical composition provided herein along with an inhibitor of PD-1 or PD-L1 produces a synergistic anti-cancer effect.

In some embodiments, an inhibitor of PD-1 or PD-L1 is an antibody or an antigen-binding portion thereof that disrupts the interaction between the human PD-1 receptor and its ligand, human PD-L1. Antibodies known in the art which bind to PD-1 and disrupt the interaction between the PD-1 and its ligand, PD-L1, and stimulate an anti-tumor immune response, are suitable for use in the methods disclosed herein. In some embodiments, the antibody or antigen-binding portion thereof binds specifically to or targets PD-1. For example, antibodies that target PD-1 include, e.g., nivolumab (BMS-936558, Bristol-Myers Squibb), pembrolizumab (lambrolizumab, MK03475, Merck), dostarlimab, tislelizumab and cemiplimab. Other suitable antibodies for use in the methods disclosed herein are anti-PD-1 antibodies disclosed in U.S. Pat. No. 8,008,449, herein incorporated by reference in its entirety. In certain embodiments, the antibody or antigen-binding portion thereof binds specifically to or targets PD-L1 and inhibits its interaction with PD-1. Antibodies known in the art which bind to PD-L1 and disrupt the interaction between the PD-1 and PD-L1, and stimulates an anti-tumor immune response, are suitable for use in the methods disclosed herein. For example, antibodies that target PD-L1 include BMS-936559 (also known as MDX 1105, Bristol-Myers Squibb), atezolizumab (Genentech), durvalumab (AstraZeneca) and avelumab (MSB0010718C). Other suitable antibodies that target PD-L1 are disclosed in U.S. Pat. No. 7,943,743, herein incorporated by reference in its entirety. Any antibody that binds specifically to PD-1 or PD-L1, disrupts the PD-1/PD-L1 interaction, and stimulates an anti-tumor immune response, is suitable for use in the methods disclosed herein.

As used herein, the term “effective amount” or “therapeutically effective amount” refers to the amount of a pharmaceutical agent, e.g., an anti-VISTA antibody or an antigen-binding portion thereof, which is sufficient to reduce or ameliorate the severity and/or duration of a disease, e.g., cancer, or one or more symptoms thereof, prevent the advancement of a disease, cause regression of a disease, prevent the recurrence, development, onset or progression of one or more symptoms associated with a disease, detect a disease, or enhance or improve the prophylactic or therapeutic effect(s) of another related therapy (e.g., prophylactic or therapeutic agent) for a VISTA-mediated disease.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are well known in the art (Ledermann J. A. et al., 1991, Int. J. Cancer 47: 659-664; Bagshawe K. D. et al., 1991, Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g., whole antibody, fragment) and the nature of any detectable label or other molecule attached to the antibody.

A typical antibody dose will be in the range 100 μg to 1 g for systemic applications, and 1 μg to 1 mg for intradermal injection. An initial higher loading dose, followed by one or more lower doses, may be administered. In some embodiments, the antibody is a whole antibody, e.g., the IgG1 or IgG4 isotype. This is a dose for a single treatment of an adult subject, which may be proportionally adjusted for children and infants, and also adjusted for other antibody formats in proportion to molecular weight. Treatments may be repeated at daily, twice-weekly, weekly or monthly intervals, at the discretion of the physician. The treatment schedule for a subject may be dependent on the pharmacokinetic and pharmacodynamic properties of the antibody composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, anti-VISTA antibody molecules and antigen-binding portions as described herein may be administered as subcutaneous injections.

In some embodiments, the therapeutic effect of an anti-VISTA antibody or an antigen-binding portion thereof may persist for several half-lives, depending on the dose. For example, the therapeutic effect of a single dose of an anti-VISTA antibody or an antigen-binding portion thereof may persist in a subject for 1 month or more, 2 months or more, 3 months or more, 4 months or more, 5 months or more, or 6 months or more.

In some embodiments, a subject is a human, a non-human primate, a cynomolgus monkey, a pig, a horse, a cow, a dog, a cat, a guinea pig, a mouse or a rat. In some embodiments, a subject is an adult human. In some embodiments, a subject is a pediatric human.

Further provided herein is an anti-VISTA antibody or an anti-VISTA antigen-binding portion, an immunoconjugate or a pharmaceutical composition described herein, for use in the treatment of a disease or a disorder (e.g., cancer).

Provided herein is an anti-VISTA antibody or an anti-VISTA antigen-binding portion, an immunoconjugate or a pharmaceutical composition described herein, for use as a medicament.

Further provided herein is a multiplexed immunohistochemistry (IHC) assay to quantify degree of spatial proximity for VISTA+ cells and PSGL-1+ cells. In some embodiments, the steps for performing such an assay are provided in Example 3. Under low pH conditions like those in the tumor microenvironment, VISTA becomes protonated, changing its charge, and likely, its shape, enabling a high-affinity interaction with PSGL-1 on T cells. PSGL-1 has been demonstrated to be a negative regulator of T cell activity akin to PD-1. In some embodiments, the assay is used to identify the tumor indication or the subject to be treated with an anti-VISTA antibody or an anti-VISTA antigen-binding portion, an immunoconjugate or a pharmaceutical composition described herein. A straightforward assessment of VISTA expression in tumors is likely to be strongly predictive as VISTA expression is necessary, but not sufficient for the checkpoint to be engaged. VISTA is strongly expressed on myeloid cells irrespective of whether VISTA is in its active, protonated form or whether it is physically interacting with PSGL-1. The assay described herein quantifies—based on multiplex IHC— the amount of PSGL-1+ cells in close physical proximity to VISTA+ cells within the tumor microenvironment.

In some embodiments, the assay comprises assigning a VISTA: PSGL-1 proximity score to a tumor sample from a subject. In some embodiments, a subject is treated with an anti-VISTA antibody or an anti-VISTA antigen-binding portion, an immunoconjugate or a pharmaceutical composition described herein if the subject's tumor sample has a VISTA: PSGL-1 proximity score above a specified threshold.

Provided herein is a method for treating a tumor in a subject, the method comprising:
(a) obtaining a VISTA:PSGL-1 proximity score for a tumor sample from the subject; wherein the VISTA:PSGL-1 proximity score is been assigned to the tumor sample by a process which comprises:
(i) obtaining an image of tissue that has been removed from the tumor sample and immunohistochemically stained for VISTA and PSGL-1 expression in a manner that allows stained VISTA cells to be distinguished from stained PSGL-1 cells;
(ii) defining in the image one or more regions of interest (ROIs) that comprises neoplastic cells and associated stroma, wherein substantially all of the neoplastic cells and associated stroma in the image are contained in the defined ROIs;
(iii) randomly creating across each defined ROI a plurality of subregions of substantially the same shape and size, wherein each of the subregions defines an area that is large enough to include a spatially proximal pair of a stained VISTA cell and a stained PSGL-1 cell and small enough to exclude pairs of stained VISTA and PSGL-1 cells that are not spatially proximal; and
(iv) calculating the percent of all of the subregions that are positive for both stained VISTA cells and stained PSGL-1 cells to generate the VISTA:PSGL-1 proximity score for the tumor sample,
(b) comparing the VISTA:PSGL-1 proximity score for the tumor sample with a threshold VISTA:PSGL-1 proximity score,
(c) classifying the tumor as biomarker positive or biomarker negative, wherein if the proximity score for the tumor sample is equal to or greater than the threshold proximity score, then the tumor is classified as positive for VISTA:PSGL-1 proximity biomarker, and if the obtained score is less than the threshold score, then the tumor is classified as negative for the VISTA:PSGL-1 proximity biomarker, and
(d) administering to the subject a therapeutically effective amount of an anti-VISTA antibody or antigen-binding portion, an immunoconjugate, or a pharmaceutical composition disclosed herein if the tumor is positive for the VISTA:PSGL-1 proximity biomarker.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular systems biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89: 10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | 3 | −4 | −1 | −3 | −3 | −1 | 0 | −1 | −4 | −3 | −3 |
| C | 0 | −3 | 3 | −3 | 9 | −3 | −4 | 3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | 1 | −3 | −2 | 2 | 6 | 2 | −4 | −4 | −2 | −3 | −3 | −2 | 0 | −2 | −2 | −3 | −3 |
| H | −2 | 0 | 1 | −1 | −3 | 0 | 0 | 2 | 8 | 3 | 3 | −1 | −2 | −1 | −2 | −1 | −2 | −2 | 2 | −3 |
| I | −1 | −3 | −3 | −3 | −1 | −3 | −3 | −4 | 3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

“Antibody-drug conjugate” and “immunoconjugate” refer to an antibody molecule, or antigen-binding portion thereof, including antibody derivatives, that binds to VISTA and is conjugated to cytotoxic, cytostatic and/or therapeutic agents.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by an antibody molecule, or antigen-binding portion thereof, at one or more of the antibody molecule's antigen-binding regions. Epitopes can consist of defined regions of primary secondary or tertiary protein structure and includes combinations of secondary structural units or structural domains of the target recognized by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody molecule can specifically bind as determined by any method well known in the art, for example, by conventional immunoassays, antibody competitive binding assays or by x-ray crystallography or related structural determination methods (for example, nuclear magnetic resonance spectroscopy).

The term “potency” is a measurement of biological activity and may be designated as IC₅₀, EC₅₀, or effective concentration of an antibody or antibody drug conjugate to the antigen VISTA to inhibit 50% of activity measured in a VISTA activity assay as described herein.

The term “inhibit” or “neutralize” as used herein with respect to bioactivity of an antibody disclosed herein means the ability of the antibody to substantially antagonize, prohibit, prevent, restrain, slow, disrupt, eliminate, stop, reduce or reverse for example progression or severity of that which is being inhibited including, but not limited to, a biological activity or binding interaction of the antibody molecule to VISTA.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The use of the alternative (e.g., “or”) should be understood to mean either one, both, or any combination thereof of the alternatives. As used herein, the terms “include” and “comprise” are used synonymously.

TABLE 1
Amino acid sequences of antibody 55873 (mab#2)

| Antibody name | 55873 (mab#2) | |
|---|---|---|
| Domain or Region | Sequence | SEQ ID NO |
| Heavy chain | EVQLVESGGGLVQPGGSLRLS | 1 |
| variable | CAASGFTFSSYSMNWVRQAPG | |
| region | KGLEWVSYISSSSSTIYYADS | |
| | VKGRFTISRDNAKNSLYLQMN | |
| | SLRAEDTAVYYCAREAGGVVG | |
| | RVDFQHWGQGTLVTVSS | |
| Heavy chain CDR1 | FTFSSYSMN | 54 |
| Heavy chain CDR2 | YISSSSSTIYYADSVKG | 55 |
| Heavy chain CDR3 | AREAGGVVGRVDFQH | 56 |
| Light chain | DIQMTQSPSSLSASVGDRVTI | 2 |
| variable | TCQASQDISNYLNWYQQKPGK | |
| region | APKLLIYDASNLETGVPSRFS | |
| | GSGSGTDFTFTISSLQPEDIA | |
| | TYYCQQADFLPPWTFGGGTKV | |
| | EIK | |
| Light chain CDR1 | QASQDISNYLN | 57 |
| Light chain CDR2 | DASNLET | 58 |
| Light chain CDR3 | QQADFLPPWT | 59 |

CDR sequences are underlined in variable region sequences.

TABLE 2
Amino acid sequences of antibody 67373

| Antibody name | 67373 | |
|---|---|---|
| Domain or Region | Sequence | SEQ ID NO |
| Heavy chain | EVQLVESGGGLVQPGGSLRLS | 3 |
| variable | CAASGFTFSDYSMNWVRQAPG | |
| region | KGLEWVSYIASDASTIYYADS | |
| | VKGRFTISRDNAKNSLYLQMN | |
| | SLRAEDTAVYYCAREAGGVVG | |
| | RVDFQHWGQGTLVTVSS | |
| Heavy chain CDR1 | FTFSDYSMN | 60 |
| Heavy chain CDR2 | YIASDASTIYYADSVKG | 61 |
| Heavy chain CDR3 | AREAGGVVGRVDFQH | 56 |
| Light chain | DIQMTQSPSSLSASVGDRVTI | 2 |
| variable | TCQASQDISNYLNWYQQKPGK | |
| region | APKLLIYDASNLETGVPSRFS | |
| | GSGSGTDFTFTISSLQPEDIA | |
| | TYYCQQADFLPPWTFGGGTKV | |
| | EIK | |
| Light chain CDR1 | QASQDISNYLN | 57 |
| Light chain CDR2 | DASNLET | 58 |
| Light chain CDR3 | QQADFLPPWT | 59 |

CDR sequences are underlined in variable region sequences.

TABLE 3
Amino acid sequences of antibody 67374

| Antibody name | 67374 | |
|---|---|---|
| Domain or Region | Sequence | SEQ ID NO |
| Heavy chain | EVQLVESGGGLVQPGGSLRLS | 4 |
| variable | CAASGFTFSSYPMNWVRQAPG | |
| region | KGLEWVSYISSDAYTIYYADS | |
| | VKGRFTISRDNAKNSLYLQMN | |
| | SLRAEDTAVYYCAREAGGVVG | |
| | RVDFQHWGQGTLVTVSS | |
| Heavy chain CDR1 | FTFSSYPMN | 62 |
| Heavy chain CDR2 | YISSDAYTIYYADSVKG | 63 |
| Heavy chain CDR3 | AREAGGVVGRVDFQH | 56 |
| Light chain | DIQMTQSPSSLSASVGDRVTI | 2 |
| variable | TCQASQDISNYLNWYQQKPGK | |
| region | APKLLIYDASNLETGVPSRFS | |
| | GSGSGTDFTFTISSLQPEDIA | |
| | TYYCQQADFLPPWTFGGGTKV | |
| | EIK | |
| Light chain CDR1 | QASQDISNYLN | 57 |
| Light chain CDR2 | DASNLET | 58 |
| Light chain CDR3 | QQADFLPPWT | 59 |

CDR sequences are underlined in variable region sequences.

TABLE 4
Amino acid sequences of antibody 67375

| Antibody name | 67375 | |
|---|---|---|
| Domain or Region | Sequence | SEQ ID NO |
| Heavy chain | EVQLVESGGGLVQPGGSLRLS | 5 |
| variable | CAASGFTFDDYSMNWVRQAPG | |
| region | KGLEWVSYISSDADTIYYADS | |
| | VKGRFTISRDNAKNSLYLQMN | |
| | SLRAEDTAVYYCAREAGGVLG | |
| | RVDFQHWGQGTLVTVSS | |
| Heavy chain CDR1 | FTFDDYSMN | 64 |
| Heavy chain CDR2 | YISSDADTIYYADSVKG | 65 |
| Heavy chain CDR3 | AREAGGVLGRVDFQH | 66 |
| Light chain | DIQLTQSPSSLSASVGDRVTI | 6 |
| variable | TCQASQDISNYLNWYQQKPGK | |
| region | APKLLIYDASNLGTGVPSRFS | |
| | GSGSGTDFTFTISSLQPEDIA | |
| | TYYCQQADFVPPWTFGGGTKV | |
| | EIK | |
| Light chain CDR1 | QASQDISNYLN | 57 |
| Light chain CDR2 | DASNLGT | 67 |
| Light chain CDR3 | QQADFVPPWT | 68 |

CDR sequences are underlined in variable region sequences.

TABLE 5
Amino acid sequences of antibody 67376
Antibody name 67376

| Domain or Region | Sequence | SEQ ID NO |
|---|---|---|
| Heavy chain | EVQLVESGGGLVQPGGSLRLSCAASGFTFDSYPMNWVRQAPG | 7 |
| variable region | KGLEWVSYISSDSSTIYYADSVKGRFTISRDNAKNSLYLQMN | |
| | SLRAEDTAVYYCAREAGGVVGRVDFQHWGQGTLVTVSS | |
| Heavy chain CDR1 | FTFDSYPMN | 69 |
| Heavy chain CDR2 | YISSDSSTIYYADSVKG | 70 |
| Heavy chain CDR3 | AREAGGVVGRVDFQH | 56 |
| Light chain | DIQLTQSPSSLSASVGDRVTITCQASQDISNYLNWYQQKPGK | 8 |
| variable region | APKLLIYDASNLETGVPSRFSGSGSGTDFTFTISSLQPEDIA | |
| | TYYCQQADFIPPWTFGGGTKVEIK | |
| Light chain CDR1 | QASQDISNYLN | 57 |
| Light chain CDR2 | DASNLGT | 67 |
| Light chain CDR3 | QQADFLPPWT | 71 |

CDR sequences are underlined in variable region sequences.

TABLE 6
Amino acid sequences of antibody 67377
Antibody name 67377

| Domain or Region | Sequence | SEQ ID NO |
|---|---|---|
| Heavy chain | EVQLVESGGGLVQPGGSLRLSCAASGFTFSDYSMNWVRQAPG | 9 |
| variable region | KGLEWVSYIASDASTIYYADSVKGRFTISRDNAKNSLYLQMN | |
| | SLRAEDTAVYYCAREAGGVSGRVDFQHWGQGTLVTVSS | |
| Heavy chain CDR1 | FTFSDYSMN | 60 |
| Heavy chain CDR2 | YIASDASTIYYADSVKG | 61 |
| Heavy chain CDR3 | AREAGGVSGRVDFQH | 72 |
| Light chain | DIQLTQSPSSLSASVGDRVTITCQASQDISNYLNWYQQKPGK | 10 |
| variable region | APKLLIYDASNLETGVPSRFSGSGSGTDFTFTISSLQPEDIA | |
| | TYYCQQADFVPPWTFGGGTKVEIK | |
| Light chain CDR1 | QASQDISNYLN | 57 |
| Light chain CDR2 | DASNLET | 58 |
| Light chain CDR3 | QQADFVPPWT | 68 |

CDR sequences are underlined in variable region sequences.

TABLE 7
Amino acid sequences of antibody 67378
Antibody name 67378

| Domain or Region | Sequence | SEQ ID NO |
|---|---|---|
| Heavy chain | EVQLVESGGGLVQPGGSLRLSCAASGFTFKDYSMNWVRQAPG | 11 |
| variable region | KGLEWVSYISSDADTIYYADSVKGRFTISRDNAKNSLYLQMN | |
| | SLRAEDTAVYYCAREAGGVKGRVDFQHWGQGTLVTVSS | |
| Heavy chain CDR1 | FTFKDYSMN | 73 |
| Heavy chain CDR2 | YISSDADTIYYADSVKG | 65 |
| Heavy chain CDR3 | AREAGGVKGRVDFQH | 74 |
| Light chain | DIQLTQSPSSLSASVGDRVTITCQASQDISNYLNWYQQKPGK | 12 |
| variable region | APELLIYDASNLETGVPSRFSGSGSGTDFTFTISSLQPEDIA | |
| | TYYCQQADFIPPWTFGGGTKVEIK | |
| Light chain CDR1 | QASQDISNYLN | 57 |
| Light chain CDR2 | DASNLET | 58 |
| Light chain CDR3 | QQADF IPPWT | 71 |

CDR sequences are underlined in variable region sequences.

TABLE 8
Amino acid sequences of antibody 67379
Antibody name 67379

| Domain or Region | Sequence | SEQ ID NO |
|---|---|---|
| Heavy chain | EVQLVESGGGLVQPGGSLRLSCAASGFTFDDYSMNWVRQAPG | 13 |
| variable region | KGLEWVSYISSDASSIYYADSVKGRFTISRDNAKNSLYLQMN | |
| | SLRAEDTAVYYCAREAGGVLGRVDFQHWGQGTLVTVSS | |
| Heavy chain CDR1 | FTFDDYSMN | 64 |
| Heavy chain CDR2 | YISSDASSIYYADSVKG | 75 |
| Heavy chain CDR3 | AREAGGVLGRVDFQH | 66 |
| Light chain | DIQLTQSPSSLSASVGDRVTITCQASQDISNYLNWYQQKPGK | 14 |
| variable region | APKLLIYDASNGETGVPSRFSGSGSGTDFTFTISSLQPEDIA | |
| | TYYCQQADFVPPWTFGGGTKVEIK | |
| Light chain CDR1 | QASQDISNYLN | 57 |
| Light chain CDR2 | DASNGET | 76 |
| Light chain CDR3 | QQADFVPPWT | 68 |

CDR sequences are underlined in variable region sequences.

TABLE 9
Amino acid sequences of antibody 67380
Antibody name 67380

| Domain or Region | Sequence | SEQ ID NO |
|---|---|---|
| Heavy chain | EVQLVESGGGLVQPGGSLRLSCAASGFTFDDYSMNWVRQAPG | 15 |
| variable region | KGLEWVSYISSDADTIYYADSVKGRFTISRDNAKNSLYLQMN | |
| | SLRAEDTAVYYCAREAGGVNGRVDFQHWGQGTLVTVSS | |
| Heavy chain CDR1 | FTFDDYSMN | 64 |
| Heavy chain CDR2 | YISSDADTIYYADSVKG | 65 |
| Heavy chain CDR3 | AREAGGVNGRVDFQH | 77 |
| Light chain | DIQLTQSPSSLSASVGDRVTITCQASQDISNYLNWYQQKPGK | 16 |
| variable region | APKLLIYDASNLEGGVPSRFSGSGSGTDFTFTISSLQPEDIA | |
| | TYYCQQADFVPPWTFGGGTKVEIK | |
| Light chain CDR1 | QASQDISNYLN | 57 |
| Light chain CDR2 | DASNLEG | 78 |
| Light chain CDR3 | QQADFVPPWT | 68 |

CDR sequences are underlined in variable region sequences.

TABLE 10
Amino acid sequences of antibody 67381
Antibody name 67381

| Domain or Region | Sequence | SEQ ID NO |
|---|---|---|
| Heavy chain | EVQLVESGGGLVQPGGSLRLSCAASGFTFSDYSMNWVRQAPG | 17 |
| variable region | KGLEWVSYISSDASSIYYADSVKGRFTISRDNAKNSLYLQMN | |
| | SLRAEDTAVYYCAREAGGVVGRVDFQHWGQGTLVTVSS | |
| Heavy chain CDR1 | FTFSDYSMN | 60 |
| Heavy chain CDR2 | YISSDASSIYYADSVKG | 75 |
| Heavy chain CDR3 | AREAGGVVGRVDFQH | 56 |
| Light chain | DIQLTQSPSSLSASVGDRVTITCQASQDISNYLNWYQQKPGK | 18 |
| variable region | APKLLIYDASNLETGVPSRFSGSGSGTDFTFTISRLQPEDIA | |
| | TYYCQQADFVPPWTFGGGTKVEIK | |
| Light chain CDR1 | QASQDISNYLN | 57 |
| Light chain CDR2 | DASNLET | 58 |
| Light chain CDR3 | QQADFVPPWT | 68 |

CDR sequences are underlined in variable region sequences.

TABLE 11
Amino acid sequences of antibody 67382
Antibody name 67382

| Domain or Region | Sequence | SEQ ID NO |
|---|---|---|
| Heavy chain | EVQLVESGGGLVQPGGSLRLSCAASGFTFSSYSMNWVRQAPG | 19 |
| variable region | KGLEWVSYISSDADTIYYADSVKGRFTISRDNAKNSLYLQMN | |
| | SLRAEDTAVYYCAREAGGVAGRVDFQHWGQGTLVTVSS | |
| Heavy chain CDR1 | FTFSSYSMN | 54 |
| Heavy chain CDR2 | YISSDADTIYYADSVKG | 65 |
| Heavy chain CDR3 | AREAGGVAGRVDFQH | 79 |
| Light chain | DIQLTQSPSSLSASVGDRVTITCQASQDISTYLNWYQQKPGK | 20 |
| variable region | APKLLIYDASNLETGVPSRFSGSGSGTDFTFTISSLQPEDIA | |
| | TYYCQQADFVPPWTFGGGTKVEIK | |
| Light chain CDR1 | QASQDISTYLN | 80 |
| Light chain CDR2 | DASNLET | 58 |
| Light chain CDR3 | QQADFVPPWT | 68 |

CDR sequences are underlined in variable region sequences.

TABLE 12
Amino acid sequences of antibody 55902 (mab#6)
Antibody name 55902 (mab#6)

| Domain or Region | Sequence | SEQ ID NO |
|---|---|---|
| Heavy chain | QVQLVQSGAEVKKPGSSVKVSCKASGGTFSSYAISWVRQAPG | 21 |
| variable region | QGLEWMGGIIPIFGTANYAQKFQGRVTITADESTSTAYMELS | |
| | SLRSEDTAVYYCARDMGDSSSYYDWLFDIWGQGTMVTVSS | |
| Heavy chain CDR1 | GTFSSYAIS | 81 |
| Heavy chain CDR2 | GIIPIFGTANYAQKFQG | 82 |
| Heavy chain CDR3 | ARDMGDSSSYYDWLFDI | 83 |
| Light chain | EIVMTQSPATLSVSPGERATLSCRASQSVSSSLAWYQQKPGQ | 22 |
| variable region | APRLLIYGASTRATGIPARFSGSGSGTEFTLTISSLQSEDFA | |
| | VYYCQQFDNYPPTFGGGTKVEIK | |
| Light chain CDR1 | RASQSVSSSLA | 84 |
| Light chain CDR2 | GASTRAT | 85 |
| Light chain CDR3 | QQFDNYPPT | 86 |

CDR sequences are underlined in variable region sequences.

TABLE 13
Amino acid sequences of antibody 67414
Antibody name 67414

| Domain or Region | Sequence | SEQ ID NO |
|---|---|---|
| Heavy chain | QVQLVQSGAEVKKPGSSVKVSCKASGGTFAQYAISWVRQAPG | 23 |
| variable region | QGLEWMGGIIPIDATAYYAQKFQGRVTITADESTSTAYMELS | |
| | SLRSEDTAVYYCARDMGDSSSYYDWLFDIWGQGTMVTVSS | |
| Heavy chain CDR1 | GTFAQYAIS | 87 |
| Heavy chain CDR2 | GIIPIDATAYYAQKFQG | 88 |
| Heavy chain CDR3 | ARDMGDSSSYYDWLFDI | 83 |
| Light chain | EIVMTQSPATLSVSPGERATLSCRASQSVSSSLAWYQQKPGQ | 22 |
| variable region | APRLLIYGASTRATGIPARFSGSGSGTEFTLTISSLQSEDFA | |
| | VYYCQQFDNYPPTFGGGTKVEIK | |
| Light chain CDR1 | RASQSVSSSLA | 84 |
| Light chain CDR2 | GASTRAT | 85 |
| Light chain CDR3 | QQFDNYPPT | 86 |

CDR sequences are underlined in variable region sequences.

TABLE 14
Amino acid sequences of antibody 67415
Antibody name 67415

| Domain or Region | Sequence | SEQ ID NO |
|---|---|---|
| Heavy chain | QVQLVQSGAEVKKPGSSVKVSCKASGGTFRQYAISWVRQAPG | 24 |
| variable region | QGLEWMGGIIPIFGDAYYAQKFQGRVTITADESTSTAYMELS | |
| | SLRSEDTAVYYCARDMGDSSSYYDWLFDIWGQGTMVTVSS | |
| Heavy chain CDR1 | GTFRQYAIS | 89 |
| Heavy chain CDR2 | GIIPIFGDAYYAQKFQG | 90 |
| Heavy chain CDR3 | ARDMGDSSSYYDWLFDI | 83 |
| Light chain | EIVMTQSPATLSVSPGERATLSCRASQSVSSSLAWYQQKPGQ | 22 |
| variable region | APRLLIYGASTRATGIPARFSGSGSGTEFTLTISSLQSEDFA | |
| | VYYCQQFDNYPPTFGGGTKVEIK | |
| Light chain CDR1 | RASQSVSSSLA | 84 |
| Light chain CDR2 | GASTRAT | 85 |
| Light chain CDR3 | QQFDNYPPT | 86 |

CDR sequences are underlined in variable region sequences.

TABLE 15
Amino acid sequences of antibody 67416
Antibody name 67416

| Domain or Region | Sequence | SEQ ID NO |
|---|---|---|
| Heavy chain | QVQLVQSGAEVKKPGSSVKVSCKASGGTFHNYAISWVRQAPG | 25 |
| variable region | QGLEWMGGIIPISGDAYYAQKFQGRVTITADESTSTAYMELS | |
| | SLRSEDTAVYYCARDMGDSSSYYDWLFDIWGQGTMVTVSS | |
| Heavy chain CDR1 | GTFHNYAIS | 91 |
| Heavy chain CDR2 | GIIPISGDAYYAQKFQG | 92 |
| Heavy chain CDR3 | ARDMGDSSSYYDWLFDI | 83 |
| Light chain | EIVMTQSPATLSVSPGERATLSCRASQSVSSSLAWYQQKPGQ | 22 |
| variable region | APRLLIYGASTRATGIPARFSGSGSGTEFTLTISSLQSEDFA | |
| | VYYCQQFDNYPPTFGGGTKVEIK | |
| Light chain CDR1 | RASQSVSSSLA | 84 |
| Light chain CDR2 | GASTRAT | 85 |
| Light chain CDR3 | QQFDNYPPT | 86 |

CDR sequences are underlined in variable region sequences.

TABLE 16
Amino acid sequences of antibody 67417
Antibody name 67417

| Domain or Region | Sequence | SEQ ID NO |
|---|---|---|
| Heavy chain | QVQLVQSGAEVKRPGSSVKVSCKASGGTFSNYAISWVRQAPG | 26 |
| variable region | QGLEWMGGIIPASGEAYYAQKFQGRVTITADESTSTAYMELS | |
| | SLRSEDTAVYYCARDEGDSSSYYDWLFDIWGQGTMVTVSS | |
| Heavy chain CDR1 | GTFSNYAIS | 93 |
| Heavy chain CDR2 | GIIPASGEAYYAQKFQG | 94 |
| Heavy chain CDR3 | ARDEGDSSSYYDWLFDI | 95 |
| Light chain | EIVLTQSPATLSVSPGERATLSCRASQSVSSNLAWYQQKPGQ | 27 |
| variable region | APGLLIYGASTRATGIPARFSGSGSGTEFTLTISGLQSEDFA | |
| | VYYCQQQDNYPPTFGGGTKVEIK | |
| Light chain CDR1 | RASQSVSSNLA | 96 |
| Light chain CDR2 | GASTRAT | 85 |
| Light chain CDR3 | QQQDNYPPT | 97 |

CDR sequences are underlined in variable region sequences.

TABLE 17
Amino acid sequences of antibody 67418
Antibody name 67418

| Domain or Region | Sequence | SEQ ID NO |
|---|---|---|
| Heavy chain | QVQLVQSGAEVKKPGSSVKVSCKASGGTFANDAISWVRQAPG | 28 |
| variable region | QGLEWMGGIIPITGEAYYAQKFQGRVTITADESASTAYMELS | |
| | SLRSEDTAVYYCARDVGDSSSYYDWLFDIWGQGTMVTVSS | |
| Heavy chain CDR1 | GTFANDAIS | 98 |
| Heavy chain CDR2 | GIIPITGEAYYAQKFQG | 99 |
| Heavy chain CDR3 | ARDVGDSSSYYDWLFDI | 100 |
| Light chain | EIVLTQSPATLSVSPGERATLSCRASQSVSSNLAWYQQKPGQ | 29 |
| variable region | APRLLIYGASTRRTGIPARFSGSGSGTEFTLTISSLQSEDFA | |
| | VYYCQQFDNSPPTFGGGTKVEIK | |
| Light chain CDR1 | RASQSVSSNLA | 96 |
| Light chain CDR2 | GASTRRT | 101 |
| Light chain CDR3 | QQFDNSPPT | 102 |

CDR sequences are underlined in variable region sequences.

TABLE 18
Amino acid sequences of antibody 67419
Antibody name 67419

| Domain or Region | Sequence | SEQ ID NO |
|---|---|---|
| Heavy chain | QVQLVQSGAEVKKPGSSVKVSCKASGGTFSDTAISWVRQAPG | 30 |
| variable region | QGLEWMGGIIPIFGPAYYAQKFQGRVTITADESTSTAYMELS | |
| | SLRSEDTAVYYCARDVGDSSSDYDWLFDIWGQGTMVTVSS | |
| Heavy chain CDR1 | GTFSDTAIS | 103 |
| Heavy chain CDR2 | GIIPIFGPAYYAQKFQG | 104 |
| Heavy chain CDR3 | ARDVGDSSSDYDWLFDI | 105 |
| Light chain | EIVLTQSPATLSVSPGERATLSCRASQSVSSNLAWYQQKPGQ | 31 |
| variable region | APRLLIYGGSTRATGIPARFSGSGSGTEFTLTISSLQSEDFA | |
| | VYYCQQLDNYPPTFGGGTKVEIK | |
| Light chain CDR1 | RASQSVSSNLA | 96 |
| Light chain CDR2 | GGSTRAT | 106 |
| Light chain CDR3 | QQLDNYPPT | 107 |

CDR sequences are underlined in variable region sequences.

TABLE 19
Amino acid sequences of antibody 67420
Antibody name 67420

| Domain or Region | Sequence | SEQ ID NO |
|---|---|---|
| Heavy chain | QVQLVQSGAEVKKPGSSVKVSCKASGGTFSDYAISWVRQAPG | 32 |
| variable region | QGLEWMGGIIPASGEAYYAQKFQGRVTITADESTSTAYMELS | |
| | SLRSEDTAVYYCARDVGDSSSDYDWLFDIWGQGTMVTVSS | |
| Heavy chain CDR1 | GTFSDYAIS | 108 |
| Heavy chain CDR2 | GIIPASGEAYYAQKFQG | 94 |
| Heavy chain CDR3 | ARDVGDSSSDYDWLFDI | 105 |
| Light chain | EIVLTQSPATLSVSPGERATLSCRASHSVSSNLAWYQQKPGQ | 33 |
| variable region | APRLLIYGASTRATGIPARFSGSGSGTEFTLTISSLQSEDFA | |
| | VYYCQQGDNYPPTFGGGTKVEIK | |
| Light chain CDR1 | RASHSVSSNLA | 109 |
| Light chain CDR2 | GASTRAT | 85 |
| Light chain CDR3 | QQGDNYPPT | 110 |

CDR sequences are underlined in variable region sequences.

TABLE 20
Amino acid sequences of antibody 67422

| Antibody name | 67422 | |
|---|---|---|
| Domain or Region | Sequence | SEQ ID NO |
| Heavy chain | QVQLVQSGAEVKKPGSSVKVSCKASGGTFHSDAISWVRQAPG | 34 |
| variable region | QGLEWMGGIIPASGEAYYAQKFQGRVTITADESTSTAYMELS | |
| | SLRSEDTAVYYCARDQRDSSDYYDWLFDIWGQGTMVTVSS | |
| Heavy chain CDR1 | GTFHSDAIS | 111 |
| Heavy chain CDR2 | GIIPASGEAYYAQKFQG | 94 |
| Heavy chain CDR3 | ARDQRDSSDYYDWLFDI | 112 |
| Light chain | ETVLTQSPATLSVSPGERATLSCRASQSVSSNLAWYQQKPGQ | 35 |
| variable region | APRLLIYGASTRATGIPARFSGSGSGTEFTLTISSLQSEDFA | |
| | VYYCQQFDWYPPTFGGGTKVEIK | |
| Light chain CDR1 | RASQSVSSNLA | 96 |
| Light chain CDR2 | GASTRAT | 85 |
| Light chain CDR3 | QQFDWYPPT | 113 |

CDR sequences are underlined in variable region sequences.

TABLE 21
Amino acid sequences of antibody 67423

| Antibody name | 67423 | |
|---|---|---|
| Domain or Region | Sequence | SEQ ID NO |
| Heavy chain | QVQLVQSGAEVKKPGSSVKVSCKASGGTFAQYAISWVRQAPG | 36 |
| variable region | QGLEWMGGIIPASGEAYYAQKFQGRVTITADESTSTAYMELS | |
| | SLRSEDTAVYYCARDVGDSSSWYDWLFDTWGQGTMVTVSS | |
| Heavy chain CDR1 | GTFAQYAIS | 87 |
| Heavy chain CDR2 | GIIPASGEAYYAQKFQG | 94 |
| Heavy chain CDR3 | ARDVGDSSSWYDWLFDT | 114 |
| Light chain | EIVLTQSPATLSVPPGERATLSCRASQPVSSNLAWYQQKPGQ | 37 |
| variable region | APRLLIYGASTRATGIPARFSGSGSGTEFTLTISSLQSEDFA | |
| | VYYCQQYDNYPPTFGGGTKVEIK | |
| Light chain CDR1 | RASQPVSSNLA | 115 |
| Light chain CDR2 | GASTRAT | 85 |
| Light chain CDR3 | QQYDNYPPT | 116 |

CDR sequences are underlined in variable region sequences.

TABLE 22
Amino acid sequences of antibody 67424

| Antibody name | 67424 | |
|---|---|---|
| Domain or Region | Sequence | SEQ ID NO |
| Heavy chain | QVQLVQSGAEVKKPGSSVKVSCKASGGTFSNYAISWVRQAPG | 38 |
| variable region | QGLEWMGGIIPIFGDAYYAQKFQGRVTITADESTSTAYMELS | |
| | SLRSEDTAVYYCARDVGDSSSEYDWLFDIWGQGTMVTVSS | |
| Heavy chain CDR1 | GTFSNYAIS | 93 |
| Heavy chain CDR2 | GIIPIFGDAYYAQKFQG | 90 |
| Heavy chain CDR3 | ARDVGDSSSEYDWLFDI | 117 |
| Light chain | EIVLTQSPATLSVSPGERATLSCRASRSVSSNLAWYQQKPGQ | 39 |
| variable region | APRLLIYGASTRATGIPARFSDSGSGTEFTLTISSLQSEDFA | |
| | VYYCQQFDNYPPTFGGGTKVEIK | |
| Light chain CDR1 | RASRSVSSNLA | 118 |
| Light chain CDR2 | GASTRAT | 85 |
| Light chain CDR3 | QQFDNYPPT | 86 |

CDR sequences are underlined in variable region sequences.

TABLE 23
Amino acid sequences of antibody 55872 (mab#1)

| Antibody name | 55872 (mab#1) | |
|---|---|---|
| Domain or Region | Sequence | SEQ ID NO |
| Heavy chain | QVQLVESGGGVVQPGRSLRLSCAASGFTFSSYGMHWVRQAPG | 40 |
| variable region | KGLEWVAVIWYEGSNKYYADSVKGRFTISRDNSKNTLYLQMN | |
| | SLRAEDTAVYYCARDYGSGSYPGGIDYWGQGTLVTVSS | |
| Heavy chain CDR1 | FTFSSYGMH | 119 |
| Heavy chain CDR2 | VIWYEGSNKYYADSVKG | 120 |
| Heavy chain CDR3 | ARDYGSGSYPGGIDY | 121 |
| Light chain | DIQMTQSPSSLSASVGDRVTITCQASQDISNYLNWYQQKPGK | 41 |
| variable region | APKLLIYDASNLETGVPSRFSGSGSGTDFTFTISSLQPEDIA | |
| | TYYCQQSDHYPWTFGGGTKVEIK | |
| Light chain CDR1 | QASQDISNYLN | 57 |
| Light chain CDR2 | DASNLET | 58 |
| Light chain CDR3 | QQSDHYPWT | 122 |

CDR sequences are underlined in variable region sequences.

TABLE 24
Amino acid sequences of antibody 55879 (mab#3)

| Antibody name | 55879 (mab#3) | |
|---|---|---|
| Domain or Region | Sequence | SEQ ID NO |
| Heavy chain | QVQLQESGPGLVKPSETLSLTCAVSGYSISSGYYWGWIRQPP | 42 |
| variable region | GKGLEWIGSIYHSGSTYYNPSLKSRVTISVDTSKNQFSLKLS | |
| | SVTAADTAVYYCARTRYALDFDLWGRGTLVTVSS | |
| Heavy chain CDR1 | YSISSGYYWG | 123 |
| Heavy chain CDR2 | SIYHSGSTYYNPSLKS | 124 |
| Heavy chain CDR3 | ARTRYALDFDL | 125 |
| Light chain | EIVLTQSPATLSLSPGERATLSCRASQSVSSYLAWYQQKPGQ | 43 |
| variable region | APRLLIYDASNRATGIPARFSGSGSGTDFTLTISSLEPEDFA | |
| | VYYCQQYSDHPRTFGGGTKVEIK | |
| Light chain CDR1 | RASQSVSSYLA | 126 |
| Light chain CDR2 | DASNRAT | |
| Light chain CDR3 | QQYSDHPRT | 128 |

CDR sequences are underlined in variable region sequences.

TABLE 25
Amino acid sequences of antibody 55880 (mab#4)

| Antibody name | 55880 (mab#4) | |
|---|---|---|
| Domain or Region | Sequence | SEQ ID NO |
| Heavy chain | QVQLQESGPGLVKPSETLSLTCAVSGYSISSGYYWGWIRQPP | 44 |
| variable region | GKGLEWIGSIYHSGSTYYNPSLKSRVTISVDTSKNQFSLKLS | |
| | SVTAADTAVYYCARVGHGYGDSLGPFDYWGQGTLVTVSS | |
| Heavy chain CDR1 | YSISSGYYWG | 123 |
| Heavy chain CDR2 | SIYHSGSTYYNPSLKS | 124 |
| Heavy chain CDR3 | ARVGHGYGDSLGPFDY | 129 |
| Light chain | DIQMTQSPSSLSASVGDRVTITCQASQDISNYLNWYQQKPGK | 45 |
| variable region | APKLLIYDASNLETGVPSRFSGSGSGTDFTFTISSLQPEDIA | |
| | TYYCQQVDDYPITFGGGTKVEIK | |
| Light chain CDR1 | QASQDISNYLN | 57 |
| Light chain CDR2 | DASNLET | 58 |
| Light chain CDR3 | QQVDDYPIT | 130 |

CDR sequences are underlined in variable region sequences.

TABLE 26
Amino acid sequences of antibody 55899

| Antibody name | 55899 | |
|---|---|---|
| Domain or Region | Sequence | SEQ ID NO |
| Heavy chain | QVQLVQSGAEVKKPGASVKVSCKASGYTFTSYGISWVRQAPG | 46 |
| variable region | QGLEWMGWISAYSGDTNYAQKLQGRVTMTTDTSTSTAYMELR | |
| | SLRSDDTAVYYCARDLGYYSSAALFDIWGQGTMVTVSS | |
| Heavy chain CDR1 | YTFTSYGIS | 131 |
| Heavy chain CDR2 | WISAYSGDTNYAQKLQG | 132 |
| Heavy chain CDR3 | ARDLGYYSSAALFDI | 133 |
| Light chain | EIVLTQSPATLSLSPGERATLSCRASQSVSSYLAWYQQKPGQ | 47 |
| variable region | APRLLIYDSSNRATGIPARFSGSGSGTDFTLTISSLEPEDFA | |
| | VYYCQQHDDWPPLTFGGGTKVEIK | |
| Light chain CDR1 | RASQSVSSYLA | 126 |
| Light chain CDR2 | DSSNRAT | 134 |
| Light chain CDR3 | QQHDDWPPLT | 135 |

CDR sequences are underlined in variable region sequences.

TABLE 27
Amino acid sequences of antibody 55901 (mab#5)

| Antibody name | 55901 (mab#5) | |
|---|---|---|
| Domain or Region | Sequence | SEQ ID NO |
| Heavy chain | QVQLVQSGAEVKKPGASVKVSCKASGYTFTSYGISWVRQAPG | 48 |
| variable region | QGLEWMGWISAYSGDTNYAQKLQGRVTMTTDTSTSTAYMELR | |
| | SLRSDDTAVYYCARDLPISAGPAFDIWGQGTMVTVSS | |
| Heavy chain CDR1 | YTFTSYGIS | 131 |
| Heavy chain CDR2 | WISAYSGDTNYAQKLQG | 132 |
| Heavy chain CDR3 | ARDLPISAGPAFDI | 136 |
| variable region | DIQMTQSPSSLSASVGDRVTITCQASQDISNYLNWYQQKPGK | 49 |
| Heavy chain | APKLLIYDASNLETGVPSRFSGSGSGTDFTFTISSLOPEDIA | |
| | TYYCQQSDILPFTFGGGTKVEIK | |
| Light chain CDR1 | QASQDISNYLN | 57 |
| Light chain CDR2 | DASNLET | 58 |
| Light chain CDR3 | QQSDILPFT | 137 |

CDR sequences are underlined in variable region sequences.

TABLE 28
Amino acid sequences of antibody 55932 (mab#7)

| Antibody name | 55932 (mab#7) | |
|---|---|---|
| Domain or Region | Sequence | SEQ ID NO |
| Heavy chain | EVOLVESGGGLVKPGGSLRLSCAASGFTFSSYSMNWVRQAPG | 50 |
| variable region | KGLEWVSSISSSSSYIYYADSVKGRFTISRDNAKNSLYLQMN | |
| | SLRAEDTAVYYCAREVYDPHYYGMDVWGQGTTVTVSS | |
| Heavy chain CDR1 | FTFSSYSMN | 54 |
| Heavy chain CDR2 | SISSSSSYIYYADSVKG | 138 |
| Heavy chain CDR3 | AREVYDPHYYGMDV | 139 |
| Light chain | DIQMTQSPSSLSASVGDRVTITCQASQDISNYLNWYQQKPGK | 51 |
| variable region | APKLLIYDASNLETGVPSRFSGSGSGTDFTFTISSLQPEDIA | |
| | TYYCQQYNPLPTFGGGTKVEIK | |
| Light chain CDR1 | QASQDISNYLN | 57 |
| Light chain CDR2 | DASNLET | 58 |
| Light chain CDR3 | QQYNPLPT | 140 |

CDR sequences are underlined in variable region sequences.

TABLE 29
Amino acid sequences of antibody 55934 (mab#8)

| Antibody name | 55934 (mab#8) | |
|---|---|---|
| Domain or Region | Sequence | SEQ ID NO |
| Heavy chain | QVQLVQSGAEVKKPGSSVKVSCKASGGTFSSYAISWVRQAPG | 52 |
| variable region | QGLEWMGGIIPIFGTANYAQKFQGRVTITADESTSTAYMELS | |
| | SLRSEDTAVYYCARGDYDDYYMDVWGKGTTVTVSS | |
| Heavy chain CDR1 | GTFSSYAIS | 81 |
| Heavy chain CDR2 | GIIPIFGTANYAQKFQG | 82 |
| Heavy chain CDR3 | ARGDYDDYYMDV | 141 |
| Light chain | DIQMTQSPSTLSASVGDRVTITCRASQSISSWLAWYQQKPGK | 53 |
| variable region | APKLLIYDASSLESGVPSRFSGSGSGTEFTLTISSLQPDDFA | |
| | TYYCQQANSHWTFGGGTKVEIK | |
| Light chain CDR1 | RASQSISSWLA | 142 |
| Light chain CDR2 | DASSLES | 143 |
| Light chain CDR3 | QQANSHWT | 144 |

CDR sequences are underlined in variable region sequences.

TABLE 30
Exemplary immunoglobulin constant region amino acid sequences

| Region | Sequence | SEQ ID NO |
|---|---|---|
| Human IgG4 | ASTKGPSVFPLAPCSRSTSESTAALGCLVKDYFPEPVTVSWNSG | 145 |
| wild type | ALTSGVHTFPAVLQSSGLYSLSSVVTVPSSSLGTKTYTCNVDHK | |
| | PSNTKVDKRVESKYGPPCPSCPAPEFLGGPSVFLFPPKPKDTLM | |
| | ISRTPEVTCVVVDVSQEDPEVQFNWYVDGVEVHNAKTKPREEQF | |
| | NSTYRVVSVLTVLHQDWLNGKEYKCKVSNKGLPSSIEKTISKAK | |
| | GQPREPQVYTLPPSQEEMTKNQVSLTCLVKGFYPSDIAVEWESN | |
| | GQPENNYKTTPPVLDSDGSFFLYSRLTVDKSRWQEGNVFSCSVM | |
| | HEALHNHYTQKSLSLSLGK | |
| Human | ASTKGPSVFPLAPCSRSTSESTAALGCLVKDYFPEPVTVSWNSG | 146 |
| IgG4(S228P) | ALTSGVHTFPAVLQSSGLYSLSSVVTVPSSSLGTKTYTCNVDHK | |
| | PSNTKVDKRVESKYGPPCPPCPAPEFLGGPSVFLFPPKPKDTLM | |
| | ISRTPEVTCVVVDVSQEDPEVQFNWYVDGVEVHNAKTKPREEQ | |
| | FNSTYRVVSVLTVLHQDWLNGKEYKCKVSNKGLPSSIEKTISKA | |
| | KGQPREPQVYTLPPSQEEMTKNQVSLTCLVKGFYPSDIAVEWES | |
| | NGQPENNYKTTPPVLDSDGSFFLYSRLTVDKSRWQEGNVFSCSV | |
| | MHEALHNHYTQKSLSLSLGK | |
| Human IgG1 | ASTKGPSVFPLAPSSKSTSGGTAALGCLVKDYFPEPVTVSWNSG | 147 |
| wild type | ALTSGVHTFPAVLQSSGLYSLSSVVTVPSSSLGTQTYICNVNHK | |
| | PSNTKVDKKVEPKSCDKTHTCPPCPAPELLGGPSVFLFPPKPKD | |
| | TLMISRTPEVTCVVVDVSHEDPEVKFNWYVDGVEVHNAKTKPRE | |
| | EQYNSTYRVVSVLTVLHQDWLNGKEYKCKVSNKALPAPIEKTIS | |
| | KAKGQPREPQVYTLPPSRDELTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPGK | |
| Human IgG1- | ASTKGPSVFPLAPSSKSTSGGTAALGCLVKDYFPEPVTVSWNSG | 148 |
| 3M | ALTSGVHTFPAVLQSSGLYSLSSVVTVPSSSLGTQTYICNVNHK | |
| | PSNTKVDKKVEPKSCDKTHTCPPCPAPEAAGAPSVFLFPPKPKD | |
| | TLMISRTPEVTCVVVDVSHEDPEVKFNWYVDGVEVHNAKTKPRE | |
| | EQYNSTYRVVSVLTVLHQDWLNGKEYKCKVSNKALPAPIEKTIS | |
| | KAKGQPREPQVYTLPPSRDELTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPGK | |
| Human IgG2 | ASTKGPSVFPLAPCSRSTSESTAALGCLVKDYFPEPVTVSWNSG | 149 |
| wild type | ALTSGVHTFPAVLQSSGLYSLSSVVTVPSSNFGTQTYTCNVDHK | |
| | PSNTKVDKTVERKCCVECPPCPAPPVAGPSVFLFPPKPKDTLMI | |
| | SRTPEVTCVVVDVSHEDPEVQFNWYVDGVEVHNAKTKPREEQFN | |
| | STFRVVSVLTVVHQDWLNGKEYKCKVSNKGLPAPIEKTISKTKG | |
| | QPREPQVYTLPPSREEMTKNQVSLTCLVKGFYPSDISVEWESNG | |
| | QPENNYKTTPPMLDSDGSFFLYSKLTVDKSRWQQGNVFSCSVMH | |
| | EALHNHYTQKSLSLSPGK | |

TABLE 31
Exemplary VISTA amino acid sequences

| Protein | Sequence | SEQ ID NO |
|---|---|---|
| Human VISTA | MGVPTALEAGSWRWGSLLFALFLAASLGPVAAFKVATPYSLYVC | 150 |
| precursor (NCBI | PEGQNVTLTCRLLGPVDKGHDVTFYKTWYRSSRGEVQTCSERRP | |
| Reference | IRNLTFQDLHLHHGGHQAANTSHDLAQRHGLESASDHHGNFSIT | |
| Sequence | MRNLTLLDSGLYCCLVVEIRHHHSEHRVHGAMELQVQTGKDAPS | |
| NP_071436.1) | NCVVYPSSSQDSENITAAALATGACIVGILCLPLILLLVYKQRQ | |
| (signal peptide | AASNRRAQELVRMDSNIQGIENPGFEASPPAQGIPEAKVRHPLS | |
| underlined) | YVAQRQPSESGRHLLSEPSTPLSPPGPGDVFFPSLDPVPDSPNF | |
| | EVI | |
| Cynomolgus | MGVPTAPEAGCWRWGSLLFALFLAASLAFKVATLYSLYVCPEGQ | 151 |
| VISTA | NVTLTCRFFGPVDKGHDVTFYKTWYRSSRGEVQTCSERRPIRNL | |
| (UniProtKB - | TFQDLHLHHGGHQAANTSHDLAQRHGLESASDHHGNFSITMRNL | |
| A0A2K5UV12) | TLLDSGLYCCLVVEIRHHHSEHRVHGAMELQVQTGKDAPSSCVA | |
| | YPSSSQESENITAAALATGACIVGILCLPLILLLVYKQRQAASN | |
| | RRAQELVRMDSNTQGIENPGFEASSPAQGILEAKVRHPLSYVAQ | |
| | RQPSESGRHLLSEPGTPLSPPGPGDVFFPSLDPVPDSPNFEVI | |

TABLE 32
Exemplary VISTA antigen amino acid sequences

| Protein | Sequence | SEQ ID NO |
|---|---|---|
| Human VISTA- | FKVATPYSLYVCPEGQNVTLTCRLLGPVDKGHDVTFYKTWYRSS | 152 |
| Fc | RGEVQTCSERRPIRNLTFQDLHLHHGGHQAANTSHDLAQRHGLE | |
| | SASDHHGNFSITMRNLTLLDSGLYCCLVVEIRHHHSEHRVHGAM | |
| | ELQVQTGKDAPSNCVVYPSSSQDSENIRSVECPPCPAPPVAGPS | |
| | VFLFPPKPKDTLMISRTPEVTCVVVDVSHEDPEVKFNWYVDGVE | |
| | VHNAKTKPREEQYNSTYRVVSVLTVLHQDWLNGKEYKCKVSNKA | |
| | LPAPIEKTISKAKGQPREPQVYTLPPSREEMTKNQVSLTCLVKG | |
| | FYPSDIAVEWESNGQPENNYKTTPPVLDSDGSFFLYSKLTVDKS | |
| | RWQQGNVFSCSVMHEALHNHYTQKSLSLSPGK | |
| Cynomolgus | FKVATLYSLYVCPEGQNVTLTCRVFGPVDKGHDVTFYKTWYRSS | 153 |
| VISTA-Fc | RGEVQTCSERRPIRNLTFQDLHLHHGGHQAANTSHDLAQRHGLE | |
| | SASDHHGNFSITMRNLTLLDSGLYCCLVVEIRHHHSEHRVHGAM | |
| | ELQVQTGKDAPSSCVAYPSSSQESENIRSVECPPCPAPPVAGPS | |
| | VFLFPPKPKDTLMIDTLMISRTPEVTCVVVDVSHEDPEVKFNWY | |
| | VDGVEVHNAKTKPREEQYNSTYRVVSVLTVLHQDWLNGKEYKCK | |
| | VSNKALPAPIEKTISKAKGQPREPQVYTLPPSREEMTKNQVSLT | |
| | CLVKGFYPSDIAVEWESNGQPENNYKTTPPVLDSDGSFFLYSKL | |
| | TVDKSRWQQGNVFSCSVMHEALHNHYTQKSLSLSPGK | |
| Mouse VISTA- | FKVTTPYSLYVCPEGQNATLTCRILGPVSKGHDVTIYKTWYLSS | 154 |
| Fc | RGEVQMCKEHRPIRNFTLQHLQHHGSHLKANASHDQPQKHGLEL | |
| | ASDHHGNFSITLRNVTPRDSGLYCCLVIELKNHHPEQRFYGSME | |
| | LQVQAGKGSGSTCMASNEQDSDSIRSVECPPCPAPPVAGPSVFL | |
| | FPPKPKDTLMISRTPEVTCVVVDVSHEDPEVKFNWYVDGVEVHN | |
| | AKTKPREEQYNSTYRVVSVLTVLHQDWLNGKEYKCKVSNKALPA | |
| | PIEKTISKAKGQPREPQVYTLPPSREEMTKNQVSLTCLVKGFYP | |
| | SDIAVEWESNGQPENNYKTTPPVLDSDGSFFLYSKLTVDKSRWQ | |
| | QGNVFSCSVMHEALHNHYTQKSLSLSPGK | |
| Human VISTA- | FKVATPYSLYVCPEGQNVTLTCRLLGPVDKGHDVTFYKTWYRSS | 155 |
| His6 | RGEVQTCSERRPIRNLTFQDLHLHHGGHQAANTSHDLAQRHGLE | |
| | SASDHHGNFSITMRNLTLLDSGLYCCLVVEIRHHHSEHRVHGAM | |
| | ELQVQTGKDAPSNCVVYPSSSQDSENIRSGGSHHHHHH | |
| Cynomolgus | FKVATLYSLYVCPEGQNVTLTCRVFGPVDKGHDVTFYKTWYRSS | 156 |
| VISTA-His6 | RGEVQTCSERRPIRNLTFQDLHLHHGGHQAANTSHDLAQRHGLE | |
| | SASDHHGNFSITMRNLTLLDSGLYCCLVVEIRHHHSEHRVHGAM | |
| | ELQVQTGKDAPSSCVAYPSSSQESENIRSGGSHHHHHH | |
| Mouse VISTA- | FKVTTPYSLYVCPEGQNATLTCRILGPVSKGHDVTIYKTWYLSS | 157 |
| Fc-His6 | RGEVQMCKEHRPIRNFTLQHLQHHGSHLKANASHDQPQKHGLEL | |
| | ASDHHGNFSITLRNVTPRDSGLYCCLVIELKNHHPEQRFYGSME | |
| | LQVQAGKGSGSTCMASNEQDSDSIRSGGSHHHHHH | |

The disclosure will be further clarified by the following examples, which are intended to be purely exemplary of the disclosure and in no way limiting.

### EXAMPLES

### Example 1. Generation and Selection of Parental pH-Dependent Anti-VISTA Antibodies

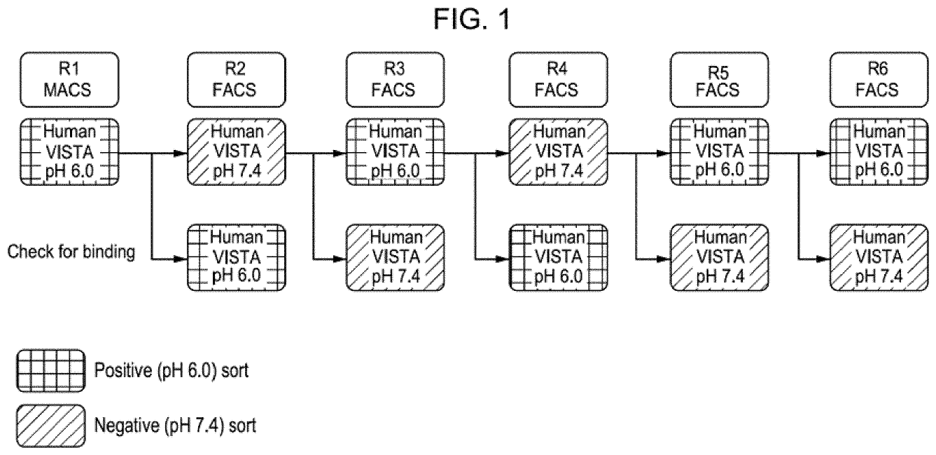

Fully human anti-VISTA antibodies were generated through pH-selective enrichment strategies of a yeast-based platform library comprising highly diverse synthetic immune repertoires. Selection of yeast-based platform libraries alternated between positive enrichment rounds at pH 6.0 and negative selection rounds at pH 7.4 (FIG. 1). Antibody-expressing yeast populations were incubated with the antigen at the specified pH, which was maintained during secondary labeling and sorting. The antigen used was the recombinant extracellular domain of human VISTA tagged with Fc or His6. VISTA-FC is a dimer, while VISTA-His6 is a monomer. Sequences of VISTA antigen proteins are provided in Table 32.

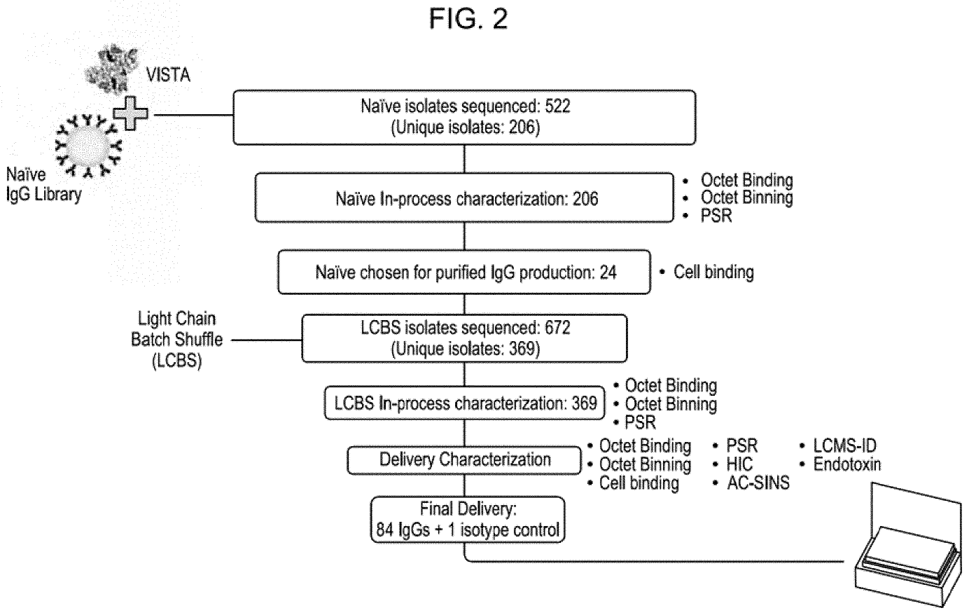

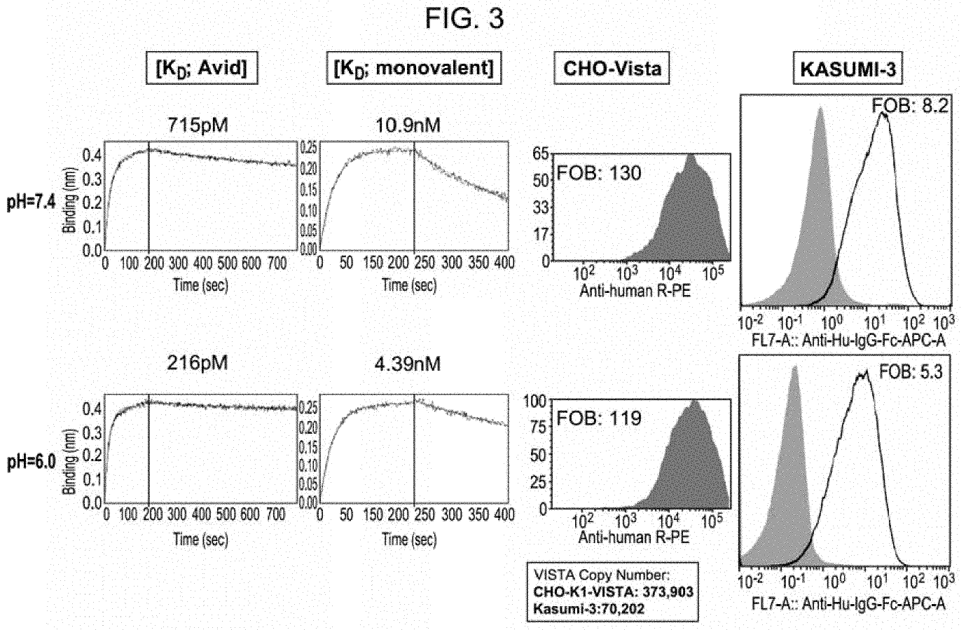

84 parental antibodies were initially isolated, based on the testing steps shown in FIG. 2. The binding characteristics of an exemplary antibody are shown in FIG. 3.

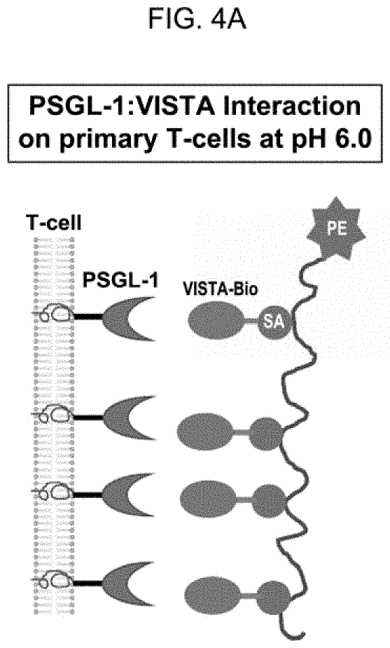

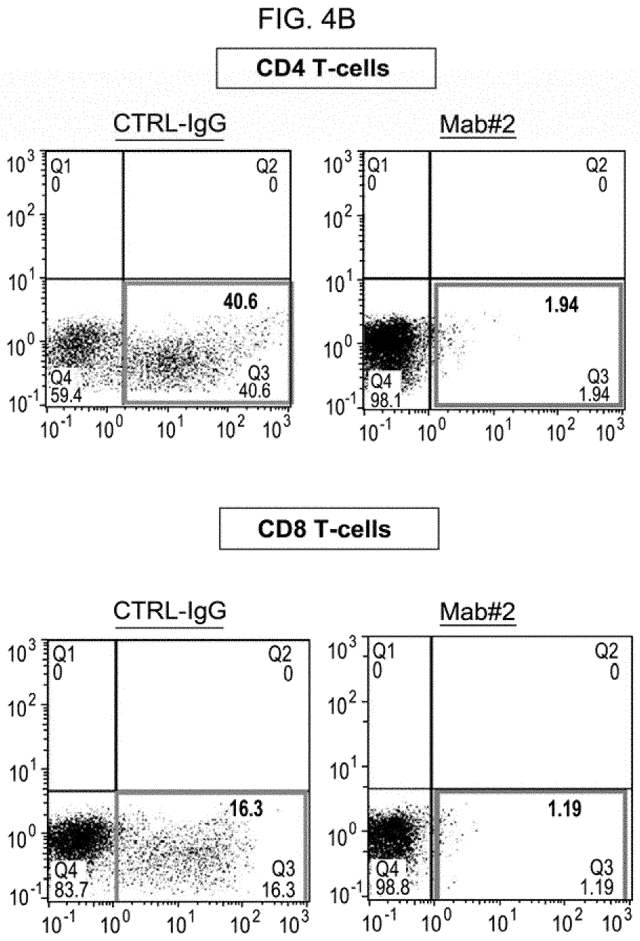

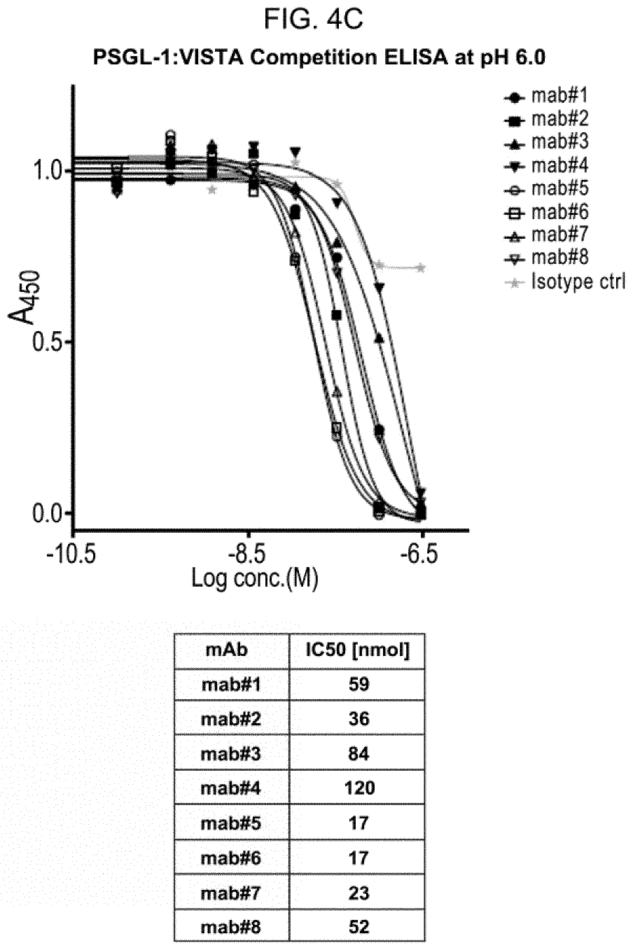

Parental antibodies were extensively characterized using flow-cytometry, Octet (ForteBio), and PSGL-1/VISTA inhibition assays in primary human CD4 and CD8 T-cells at pH 6.0 and pH 7.4 (FIG. 4A—FIG. 4C). These analyses revealed candidates displaying pH-dependent binding to endogenously expressed native VISTA on cells. PSGL-1/VISTA inhibition assays at pH 6.0 identified potent antagonists.

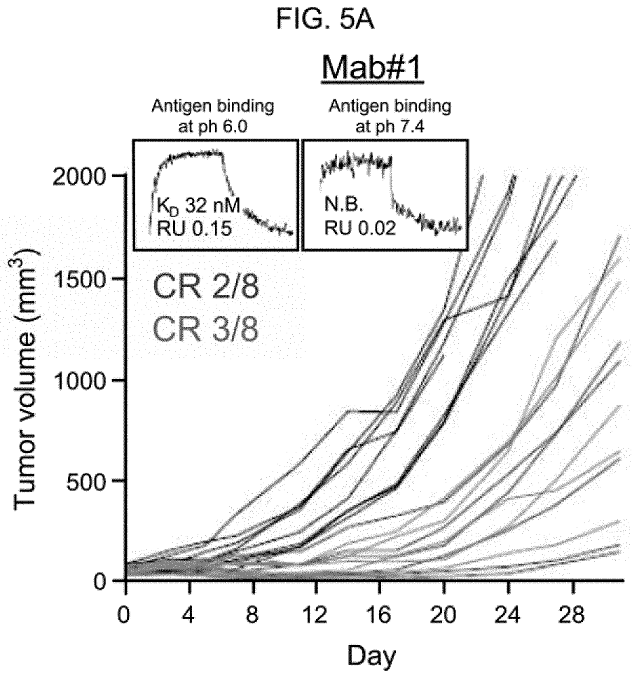

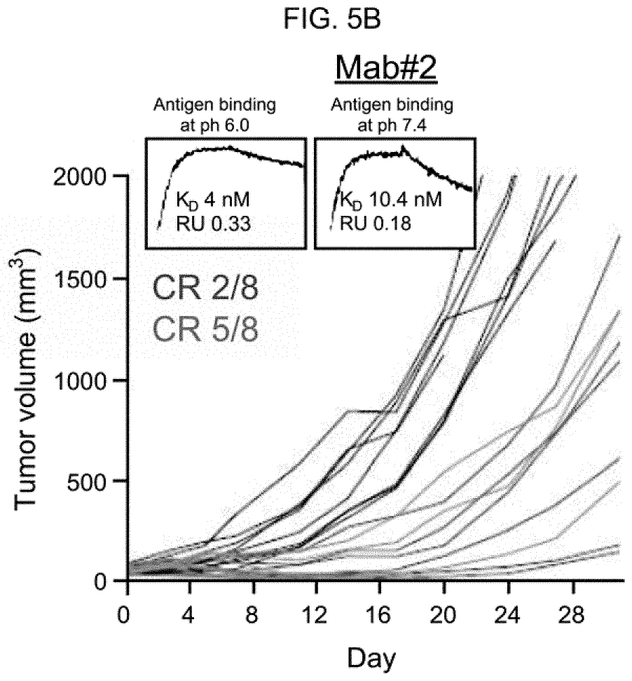

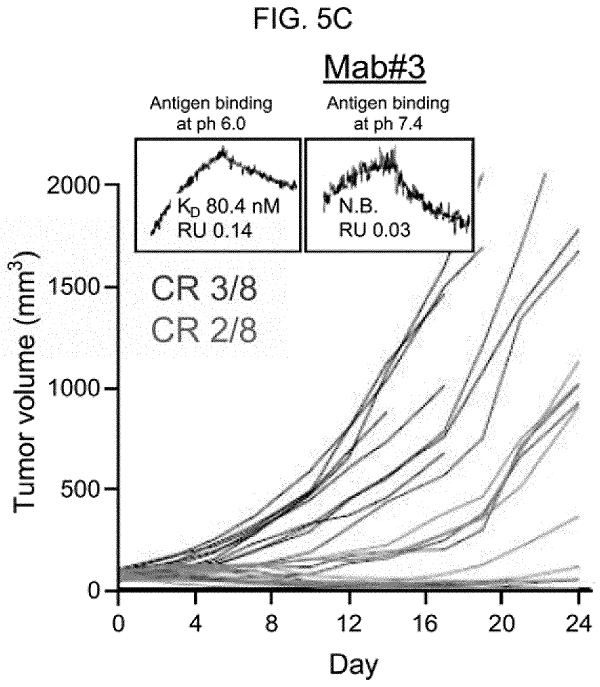

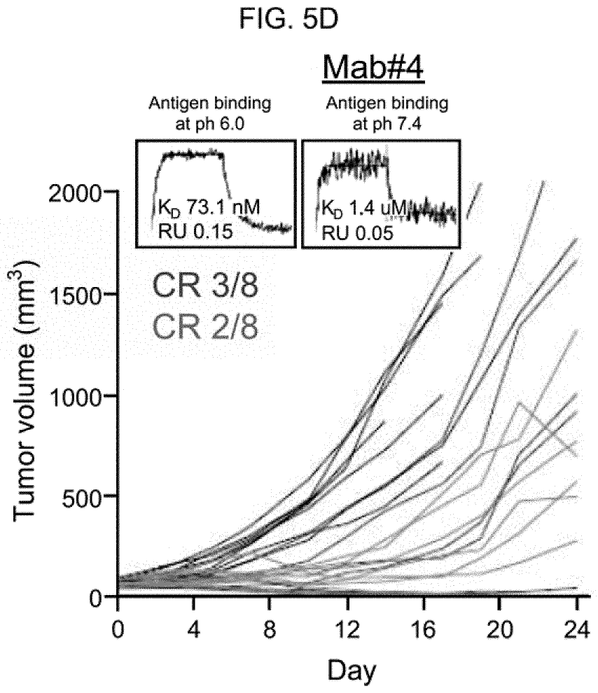

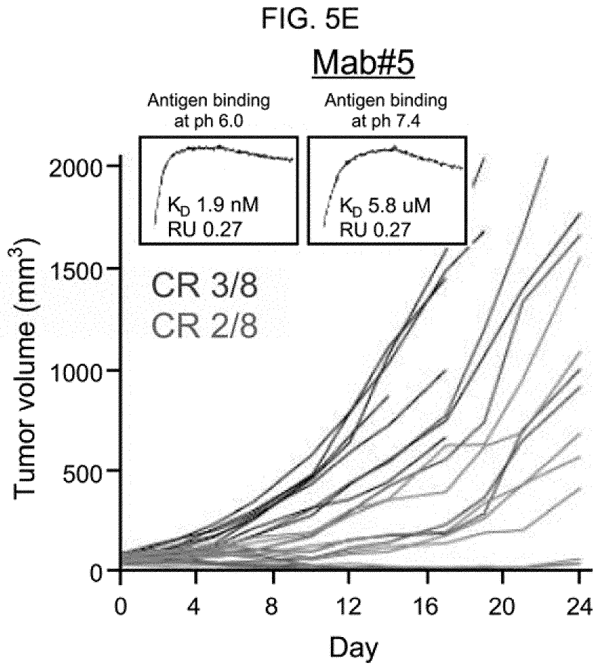

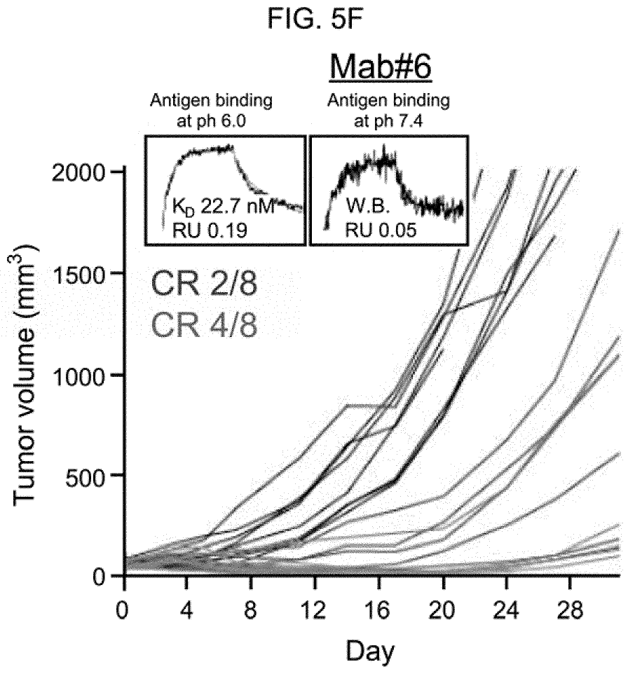

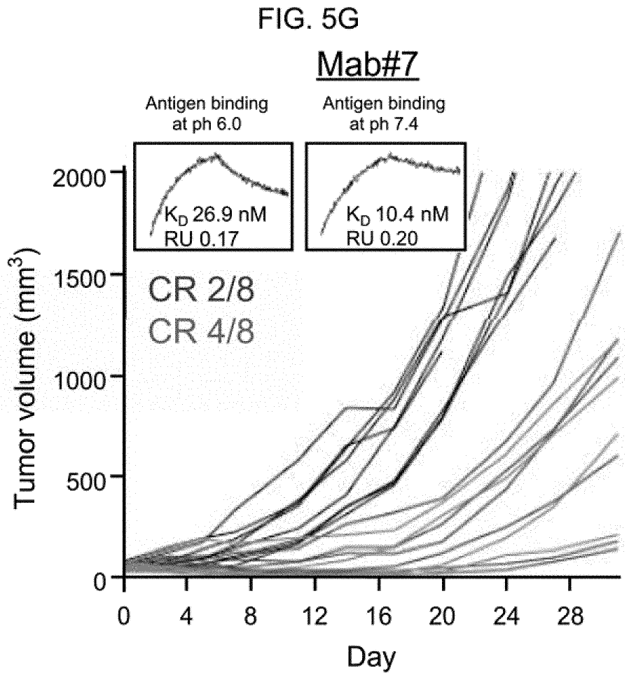

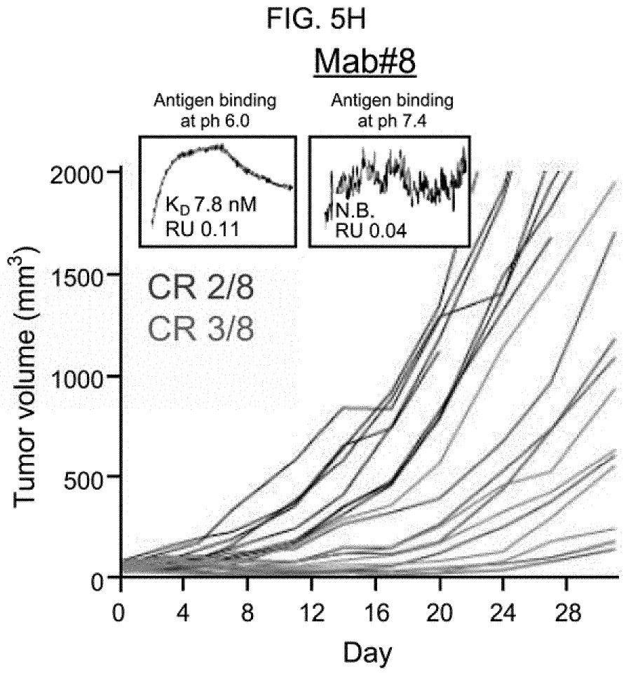

Eight parental antibodies were identified (Mab #1-Mab #8) and tested for combinatorial efficacy with anti-mouse PD-1 antibody (rat mAb clone RMP1-14) in vivo in human VISTA knock-in (KI) mice inoculated with syngeneic MC-38 tumors. 1×10⁶ MC-38 cells were implanted into female VISTA-KI mice. Mice were randomized (n=8/cohort) once tumor volumes reached ˜0.60-80 mm³. Antibodies were administered intraperitoneally twice/week for 2 weeks at 40 mg/kg total (20 mg/kg each). Results are shown in FIG. 5A—FIG. 5H. The antibodies demonstrated varied combinatorial efficacy, with a subset leading to superior tumor rejection. Optimization of the parental antibodies was carried out as described in Example 2.

### Example 2. Optimization of pH-Dependent Anti-VISTA Antibodies

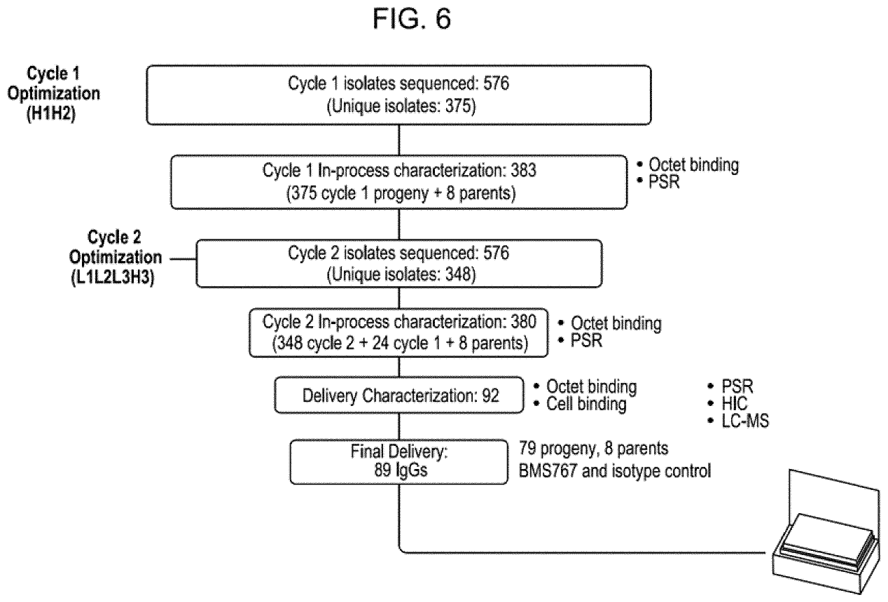

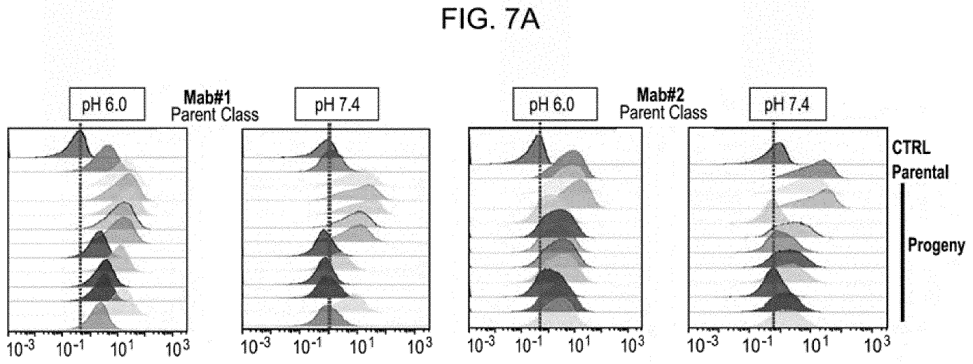

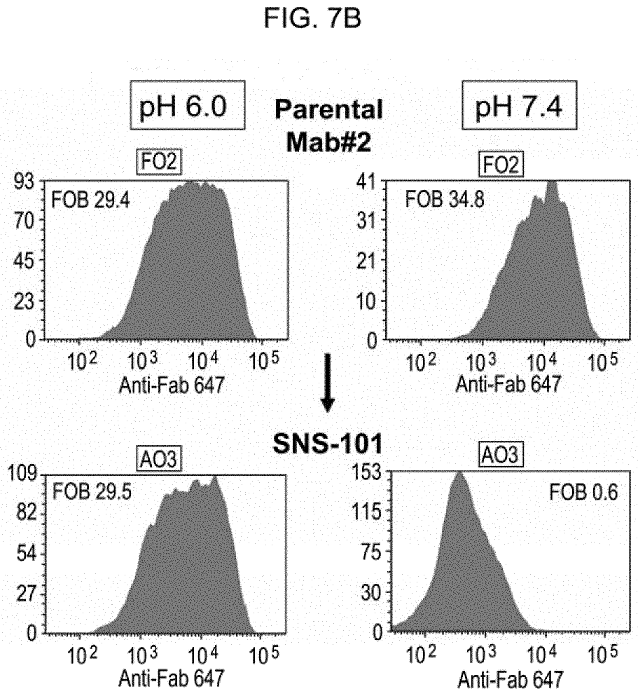

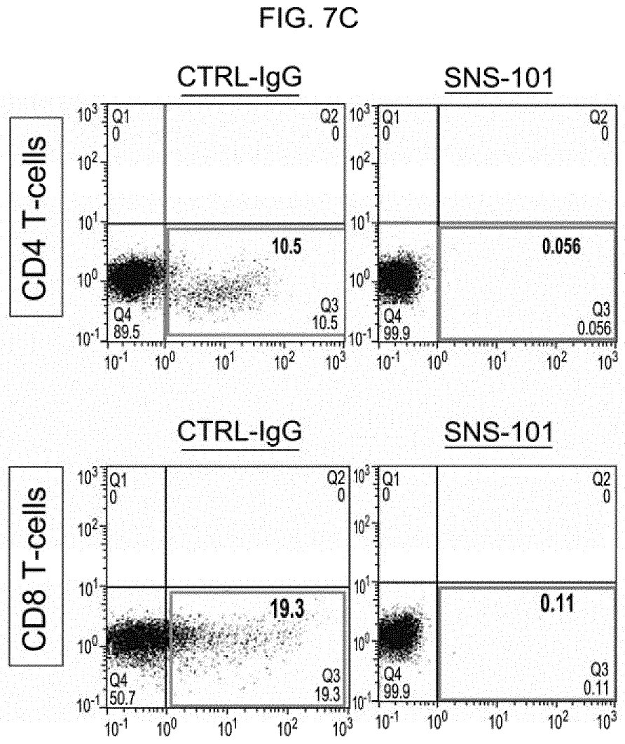

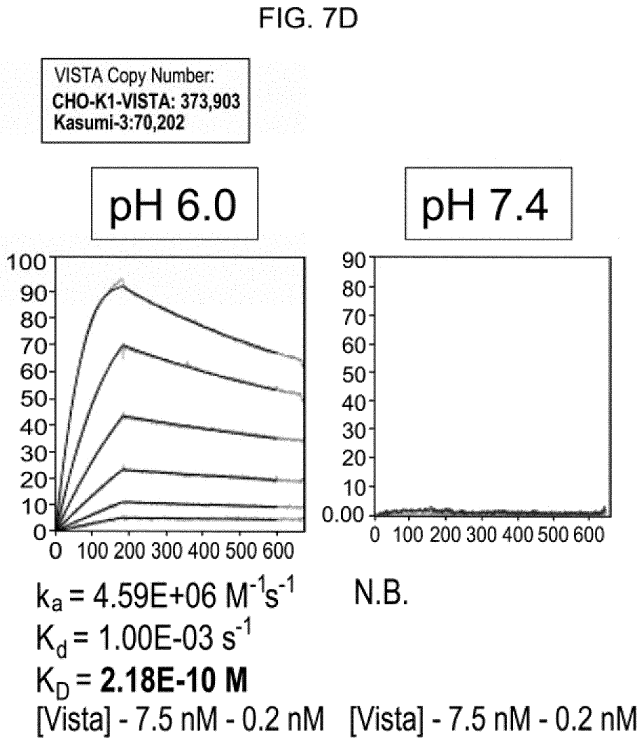

Optimization of the 8 parental antibodies for enhanced binding affinity and selectivity at pH 6.0 over pH 7.4 was performed using the steps depicted in FIG. 6. Characterization and ranking of the optimized antibodies was based on the same in vitro and in vivo techniques as of the parental antibodies. FIG. 7A—FIG. 7D show results from characterization of two representative anti-VISTA parental antibodies and their respective progeny.

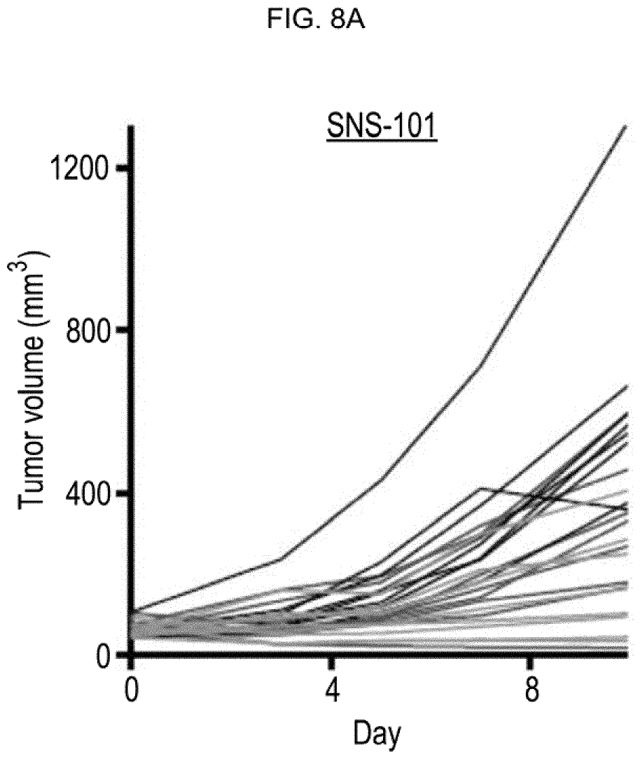

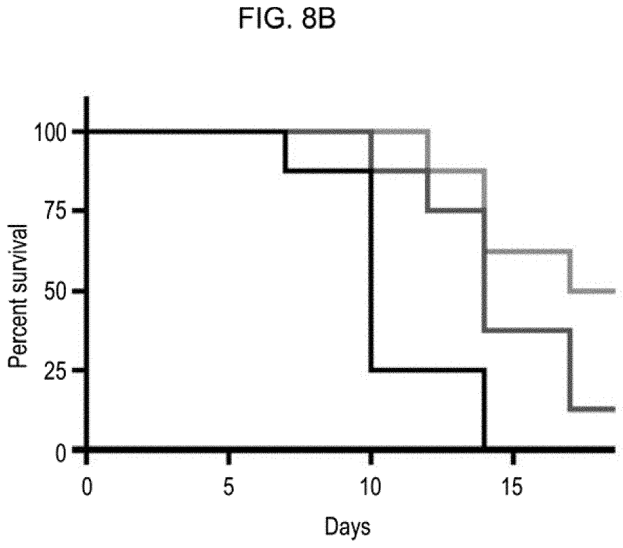

Results from characterization of optimized antibodies led to identification of antibody SNS-101, a pH-selective, high-affinity, cynomolgus monkey cross-reactive IgG1 with excellent biophysical and biochemical properties (see Table 4 for SNS-101 amino acid sequences). SNS-101 activity was tested in an in vivo mouse tumor model. 1×10⁶ MC-38 cells were implanted into female VISTA knock-in mice. Mice were randomized (n=8/cohort) once tumor volumes reached ˜0.60-80 mm³ Antibodies were administered intraperitoneally twice/week for 1 week at 40 mg/kg total (20 mg/kg each). Effects on tumor volume and percent survival are shown in FIG. 8A and FIG. 8B, respectively. The groups were treated with (1) IgG CTRL human & rat, (2) IgG CTRL human & rat anti-mPD-1, or (3) rat anti-mPD-1 & anti-VISTA.

SNS-101 had a monovalent affinity (K_{D}) at pH 6.0 of 0.218 nM and a monovalent affinity (K_{D}) at pH 7.4 of 132 nM (or “non-binding”). Further, SNS-101 inhibits PSGL-1/VISTA interaction at greater than 95%, induces tumor rejection in syngeneic tumor models as mono therapy in more than 40% of animals and induces tumor rejection in syngeneic tumor models as combinatorial therapy in about 50% of animals.

### Example 3. Multiplexed Immunohistochemistry (IHC) Assay to Quantify Degree of PSGL-1/VISTA Spatial Proximity in Tumor Cells

Multiplex IHC. Formalin-fixed paraffin-embedded tissue microarrays were purchased from US Biomax (Rockville, Md.) and baked for 30 minutes at 60° C. The slides were then dewaxed using standard IHC protocols and antigen retrieved in a Decloaker (Biocare Medical, Pacheco, Calif.) for 20 minutes in Tris-EDTA pH 9 (Abcam, Waltham, Mass.) at 95° C. before loading into a Biocare intelliPATH FLX® autostainer using TBS Automation Wash Buffer (Biocare) for wash steps.

TABLE 33
Staining Panel 5VISTA_1

| Position | Antibody* | Clone/Host | Company/Item | Concentration | OPAL Fluor (1:50) |
|---|---|---|---|---|---|
| 1 | VISTA | D1L2G/Rabbit | Cell Signaling/ | 0.2 μg/ml | 520 |
| | | | 64953S | | |
| 2 | PSGL-1 | TC2/Mouse | LSBio/LS-B4514 | 0.05 μg/ml | 570 |

| | | | |
|---|---|---|---|
| Antibody | Da Vinci Green Diluent | Biocare/PD900 | RTU |
| Diluent | | | |
| Secondary | Opal Polymer HRP Ms + Rb | Akoya Biosciences/ | RTU |
| | | ARH1001EA | |

Endogenous peroxidase was blocked with 3% H₂O₂ for 5 minutes followed by Background Punisher protein block (Biocare) for 5 minutes. The first primary antibody (position 1) was applied for 30 minutes followed by the secondary antibody application for 20 minutes and the application of the tertiary TSA-amplification reagent (OPAL fluor, Akoya Biosciences, Menlo Park, Calif.) for 15 minutes. All blocking and staining steps were performed at room temperature. The primary and secondary antibodies were stripped in the Decloaker with Tris-EDTA at 95° C. for 20 minutes before repeating the process with the second primary antibody (position 2). Slides were removed from the autostainer and stained with Spectral DAPI (Akoya) for 5 minutes, rinsed for 5 minutes, and cover-slipped with Prolong Gold Antifade reagent (Invitrogen/Life Technologies, Grand Island, N.Y.).

Imaging. Slides were cured for 24 hours at room temperature in the dark then imaged on the PhenoImager™ HT Automated Quantitative Pathology Imaging System (Akoya) and exported as QPTIFF files for image analysis.

Image Analysis. TMA (tissue microarray) segmentation and cellular analysis of the images was performed with HALO image analysis software (v3.4, Indica Labs, Corrales, N. Mex.). After the cells were visualized based on nuclear and cytoplasmic stains using the HighPlex FL v4.1.3 analysis module, the software measured mean pixel fluorescence intensity in each compartment. A mean intensity threshold above background was used to determine positivity for each fluorochrome, thereby defining cells as either positive or negative for each marker. Pathologist-reviewed DAB stain on adjacent/near sections of the TMAs for each marker was used to quality control the fluorescence stain quality and verify marker threshold cutoff.

The positive cell data was used to define single-positive and double-positive populations and perform spatial analysis on each TMA core. Proximity analysis was performed in the HALO Spatial Analysis module determining the number and average distance of PSGL-1+ cells within 100 μm of a VISTA+cell. The 100 μm distance was divided into 10 μm-wide bands around the VISTA+ cells to generate histograms as visual representations of the average number of PSGL-1 cells within each band for a qualitative view of proximity of PSGL-1 to VISTA.

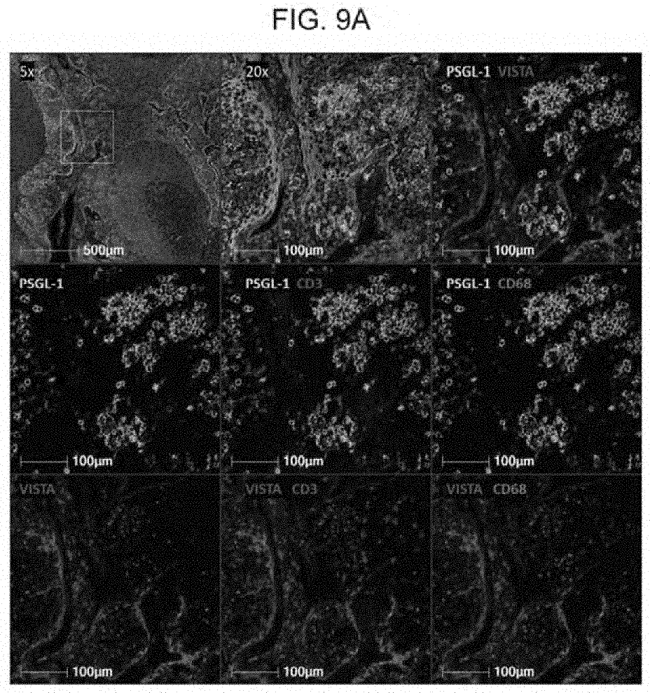

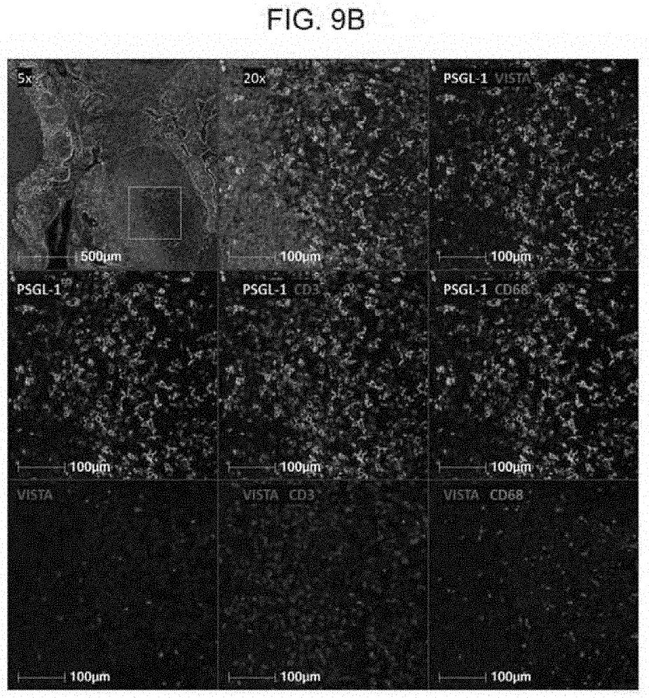

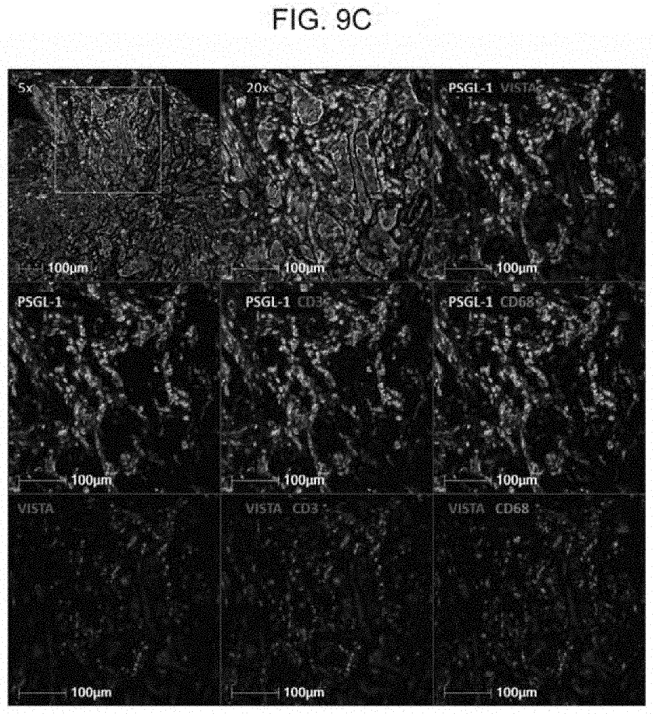

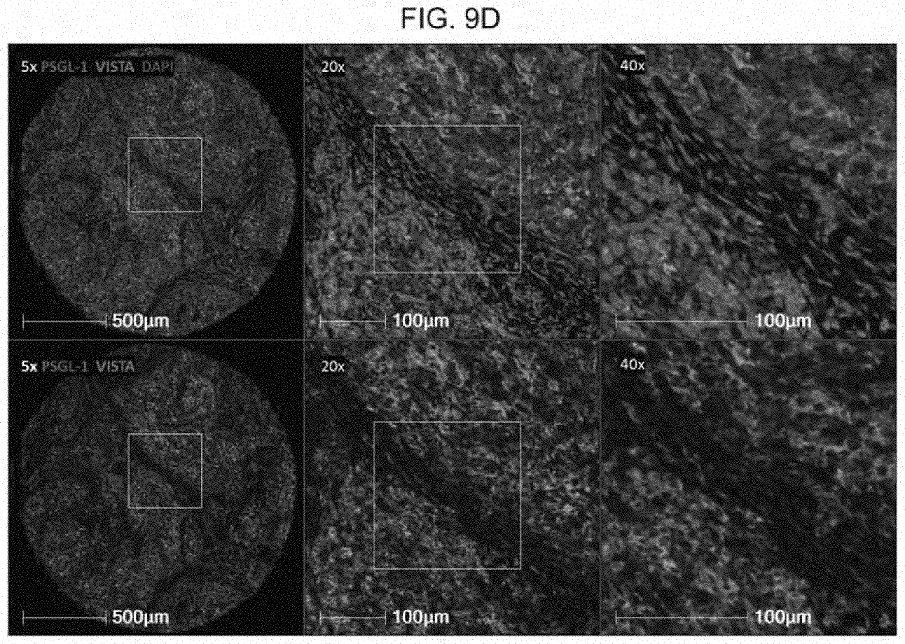

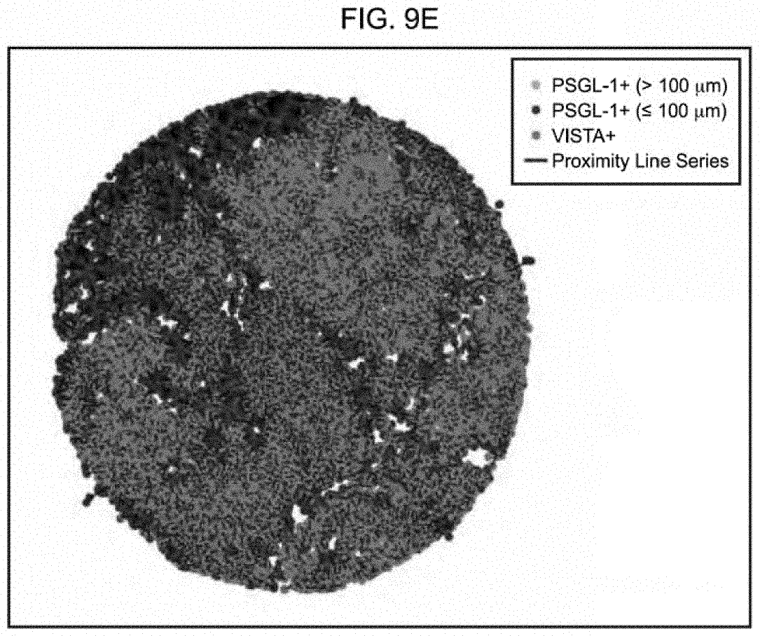

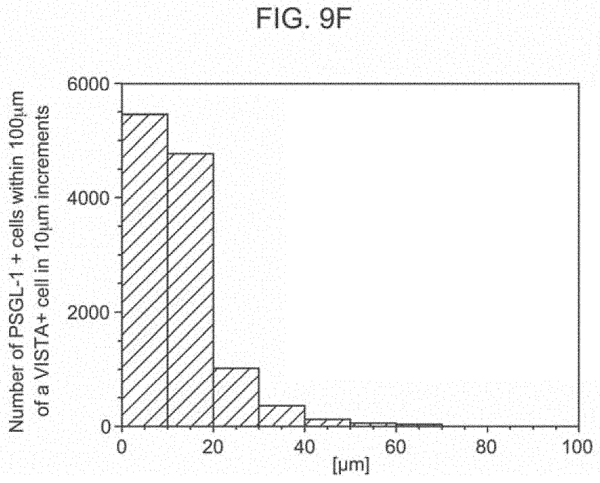

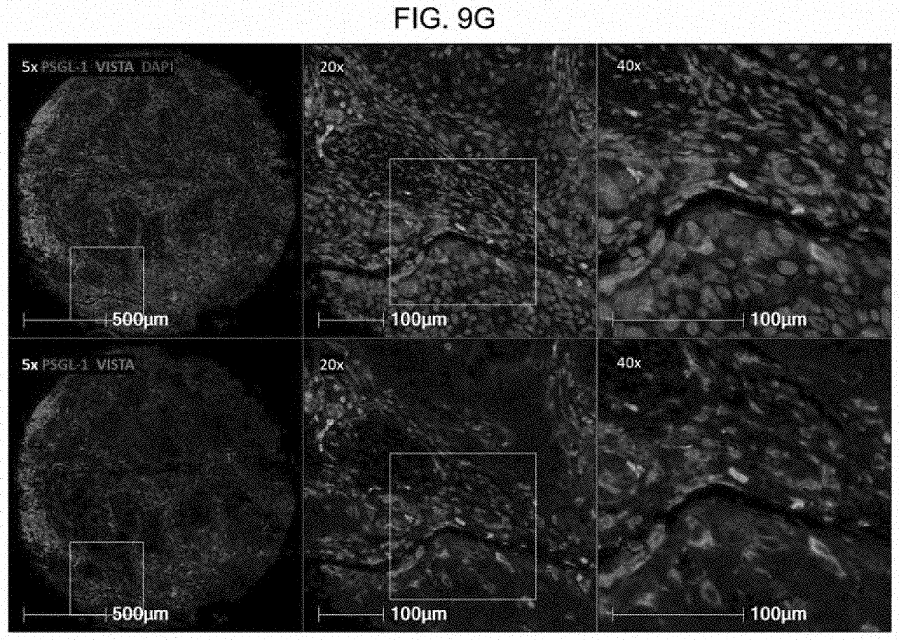

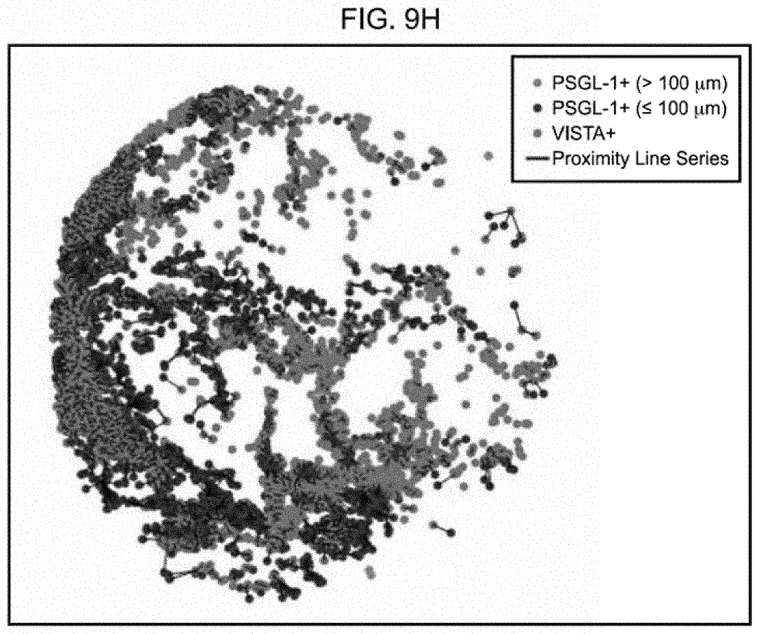

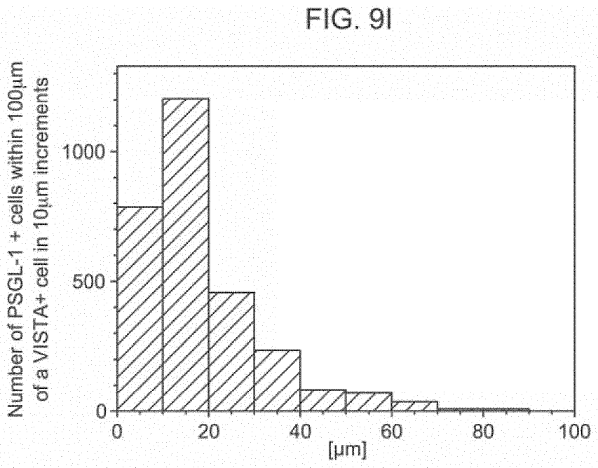

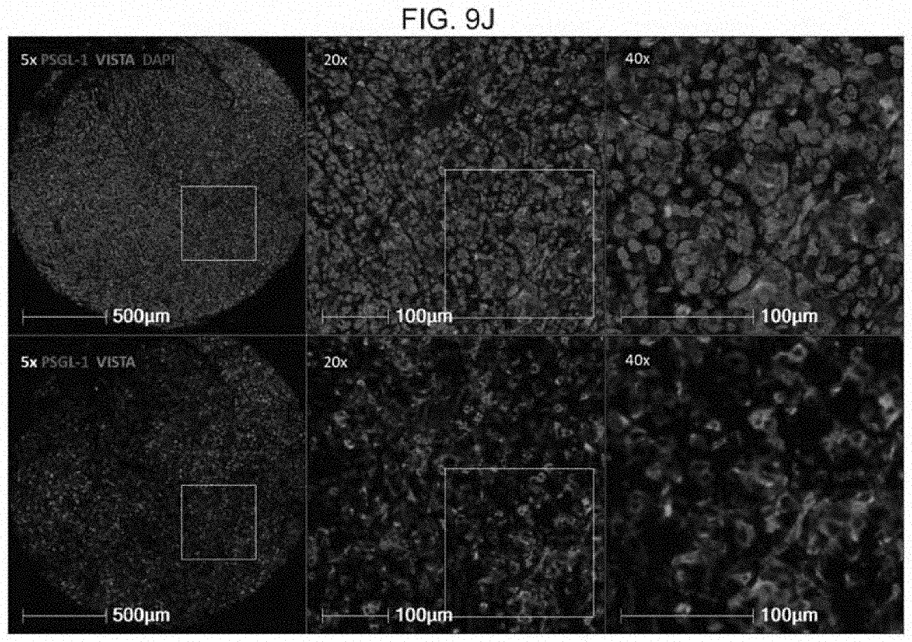

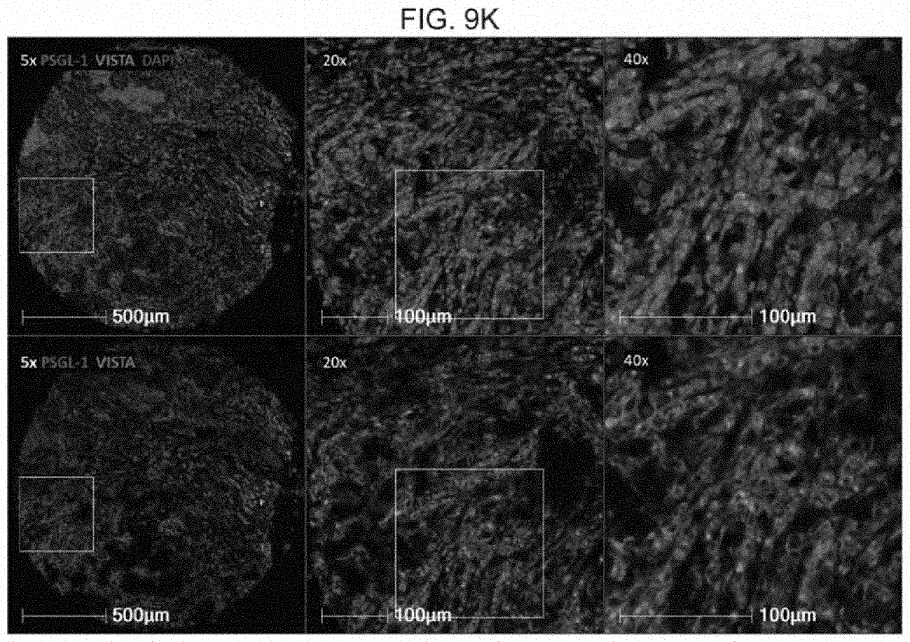

Representative images and results of data analysis are shown in FIG. 9A-FIG. 9L.

### Example 4. Cytokine Release Profile of SNS-101 in an Ex Vivo System that Mimics Human Blood Circulation

Cancer immunotherapeutics can give rise to immune-mediated adverse events (AEs), including CRS (Shimabukuro-Vornhagen et al., J Immunother Cancer. 2018; 6(1):56). On-target, off-tumor activation of myeloid lineage cells (e.g., monocytes) has been implicated as a factor in the generation of these immune-mediated AEs (Mortha et al., Front Immunol. 2018; 9:191). Antibodies binding to VISTA at physiological pH (7.4) have significant potential to induce dose-limiting toxicities such as CRS and be prematurely eliminated from circulation through target-mediated drug disposition (TMDD), reducing the likelihood of reaching efficacious drug occupancy levels. Among several non-pH-selective antibodies in clinical development, JNJ-61610588 (now CI-8993) induced dose-limiting, on-target CRS at subtherapeutic dose levels and exhibited TMDD (Curis Inc., Corporate Presentation, Jefferies Global Healthcare Conference, Jun. 9, 2022, available at the URL filecache.investorroom.com/mr5ir_curis/225/2022-06_Curis %20Corporate %20Presentation.pdf).

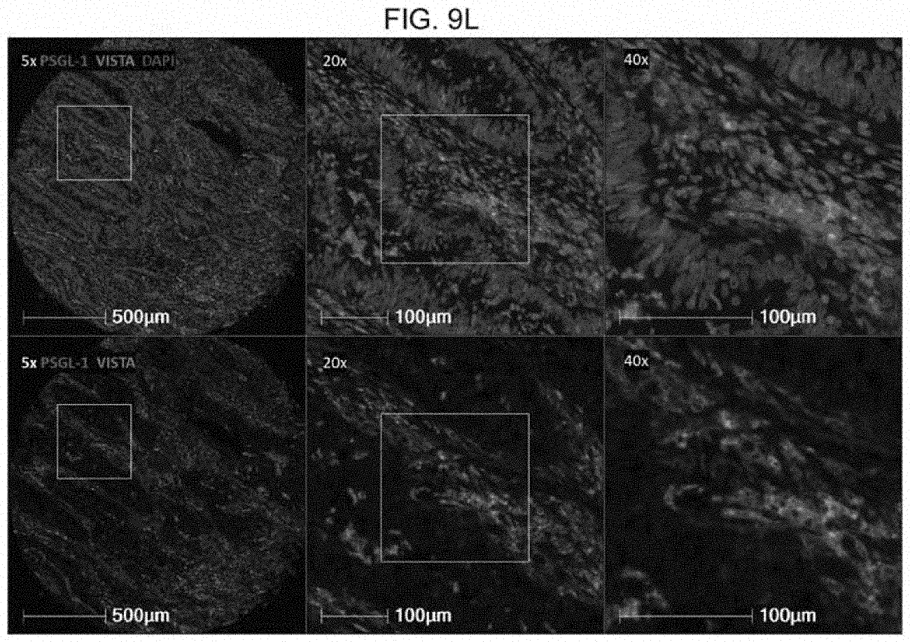

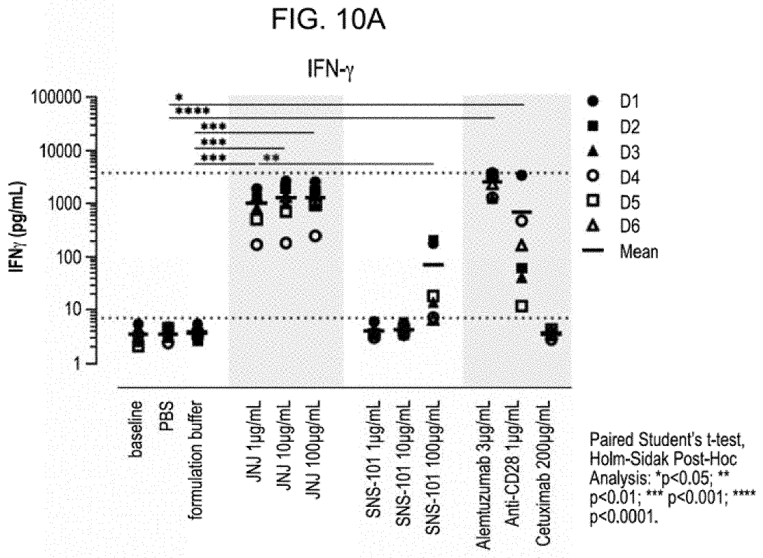

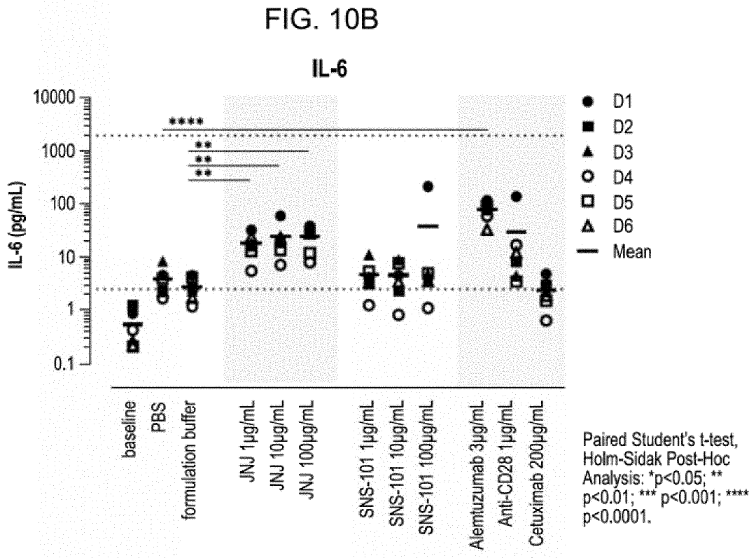

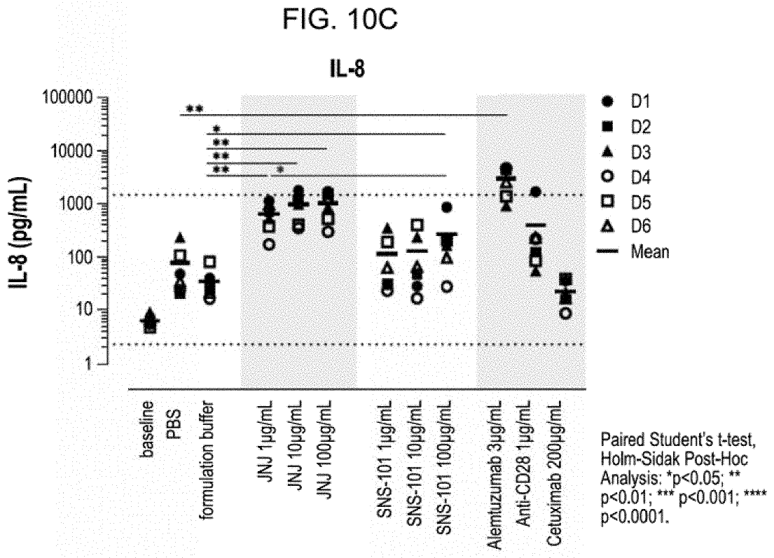

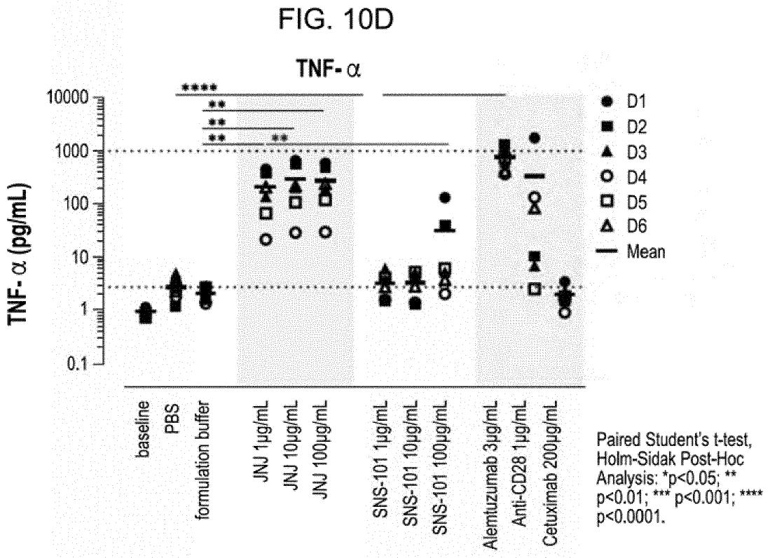

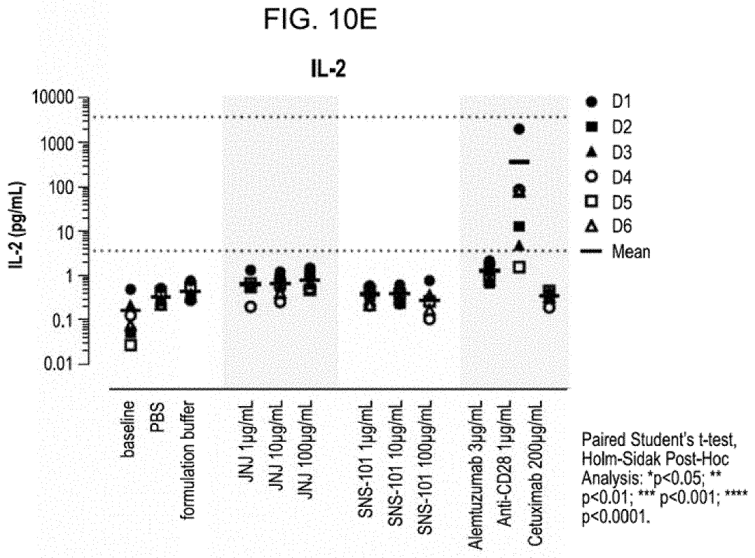

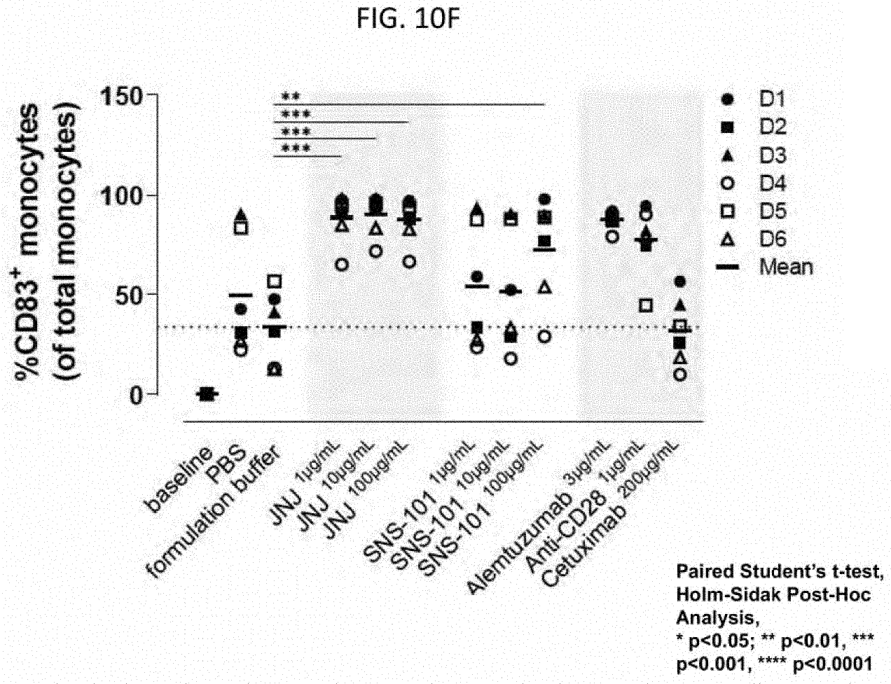

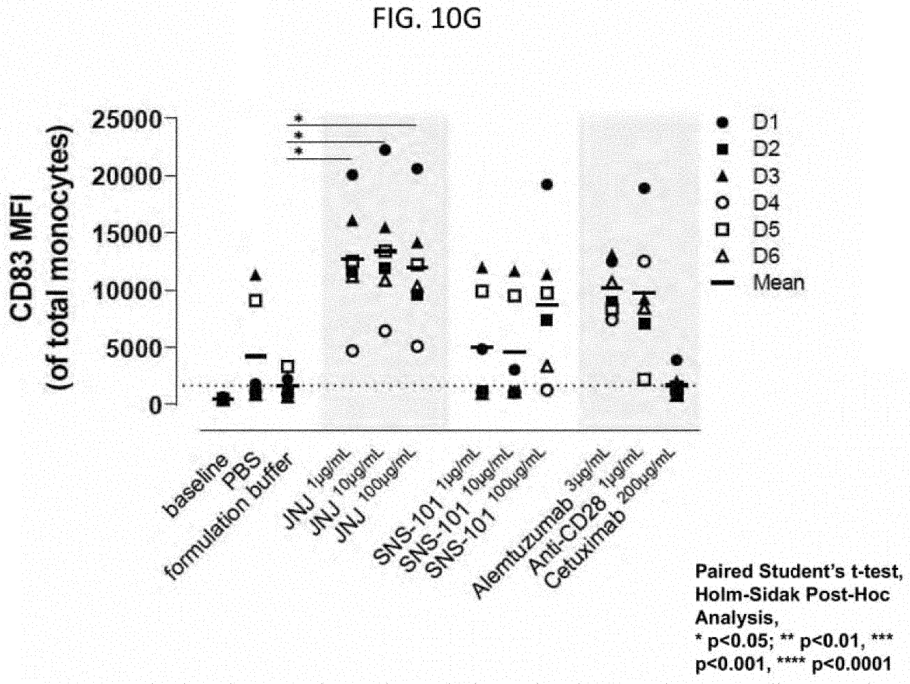

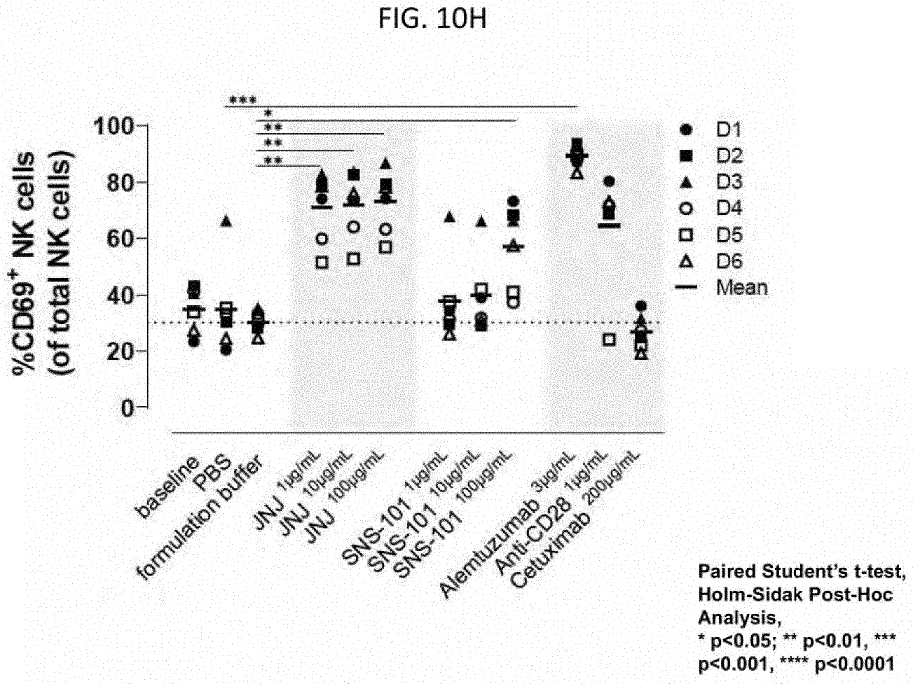

It has been shown that a circulating whole blood loop assay is a powerful human-based extra-corporal assay to predict CRS, as it is superior to a standard plate assay in presenting distinctively lowered background of cytokine release, which translates to increased sensitivity of the assay (Fletcher et al., Int Immunopharmacol. 2018; 54:1-11). Using this assay it was tested whether the selective, pH-dependent binding properties of SNS-101 would mitigate the CRS risk compared to JNJ (variable region JNJ-61610588 antibody cloned onto human IgG1 backbone) (FIG. 10A-FIG. 10E).

The study included six healthy donors. Plasma samples were collected at baseline and after 4 h from ID.Flow. Calculated lower-limit of quantitation (LLOQ) and upper-limit of quantitation (ULOQ) are marked with a dotted line. Paired Student's t-test performed on Log₁₀ transformed values (values below LLOQ were set up to LLOQ and values above ULOQ were set to ULOQ) followed by Holm-Sidak correction for multiple comparisons; Control substances alemtuzumab (positive control), anti-CD28 (positive control), and cetuximab (negative isotype control) were compared to PBS, while JNJ and SNS-101-treated samples were compared to formulation buffer, and selected JNJ- and SNS-101-treated samples were compared to each other. Statistical significance is denoted by *. * p<0.05; ** p<0.01; *** p<0.001; **** p<0.0001. Comparisons that did not reach statistical significance are not indicated. The controls alemtuzumab, anti-CD28, and cetuximab, exhibited the expected effects on cytokine release.

IFNγ, IL-6, and TNFα levels in SNS-101-treated samples were comparable to formulation buffer at lower concentrations (1 μg/mL and 10 μg/mL). SNS-101 at 100 μg/mL resulted in a mild induction of IFNγ and TNFα, which did not reach statistical significance. SNS-101-treated samples displayed elevated levels of IL-8 at all concentrations tested, and at 100 μg/mL, this induction was statistically significant compared to formulation buffer. In comparison, JNJ resulted in a statistically significant increase in IFNγ, IL-6, IL-8, and TNFα levels compared to formulation buffer at all concentrations tested. Levels for IL-2 were below LLOQ for both JNJ and SNS-101 for all concentrations tested. The lowest concentration of JNJ tested resulted in statistically significant higher levels of IFNγ, TNFα and IL-8 compared to the highest (100-fold higher) tested concentration of SNS-101. Taken together, SNS-101 displays a favorable pro-inflammatory profile compared to JNJ.

Proportions of CD83+ monocytes (FIG. 10F), CD83 MFI (median) (FIG. 10G), CD69+NK cells (FIG. 10H) and CD69 MFI (median) (FIG. 10I) were measured. Blood was collected at baseline and after 4 h from ID.Flow, followed by flowcytometry analysis. The dotted line indicates the mean for the formulation buffer group. Paired Student's t-test was performed on the values followed by Holm-Sidak correction for multiple comparisons; Control substances alemtuzumab, anti-CD28, and cetuximab were compared to PBS, while JNJ and SNS-101-treated samples were compared to formulation buffer; * p<0.05; ** p<0.01; *** p<0.001; comparisons that did not reach statistical significance are not indicated.

JNJ at all concentrations as well as the highest concentration of SNS-101 significantly activated monocytes compared to formulation buffer, whereas the remaining concentrations of SNS-101 only activated monocytes in part of the donors (FIG. 10F, FIG. 10G). CD83 MFI (monocytes) were overall higher in JNJ- than in SNS-101-treated samples (FIG. 10F, FIG. 10G).

JNJ resulted in significant NK cell activation (CD69) at all concentrations tested, while SNS-101 resulted in significant induction of NK cell activation only at the highest concentration tested (FIG. 10H, FIG. 10I). However, this did not reach a level of significance compared to formulation buffer (FIG. 10I).

### Example 5. Pharmacokinetic Profile of SNS-101 in Cynomolgus Monkey

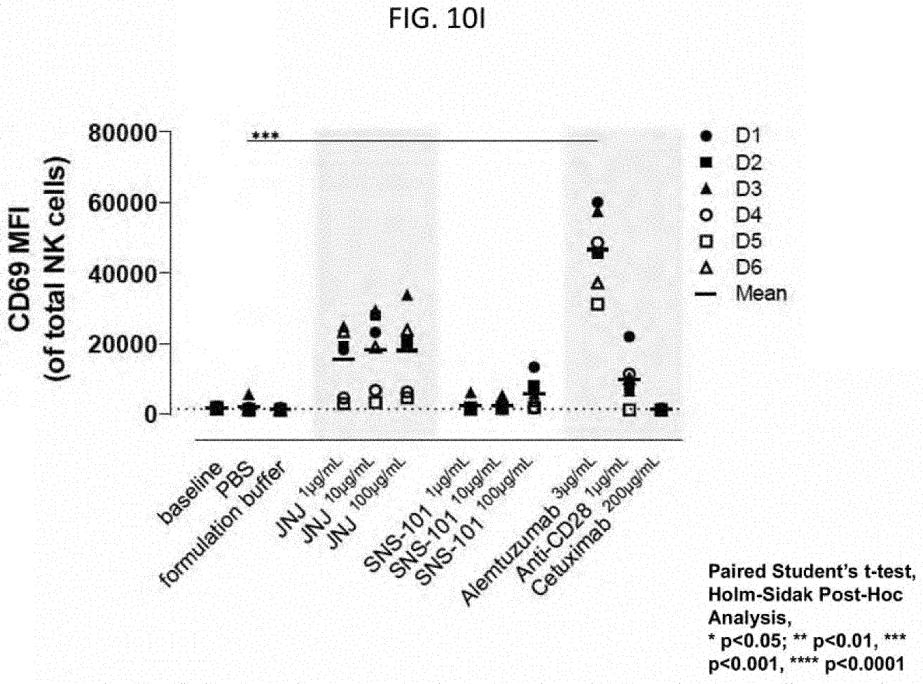

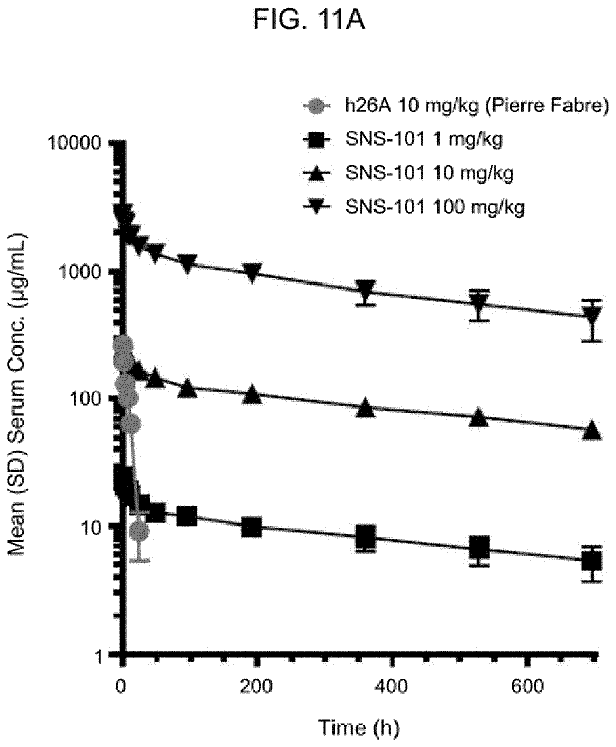

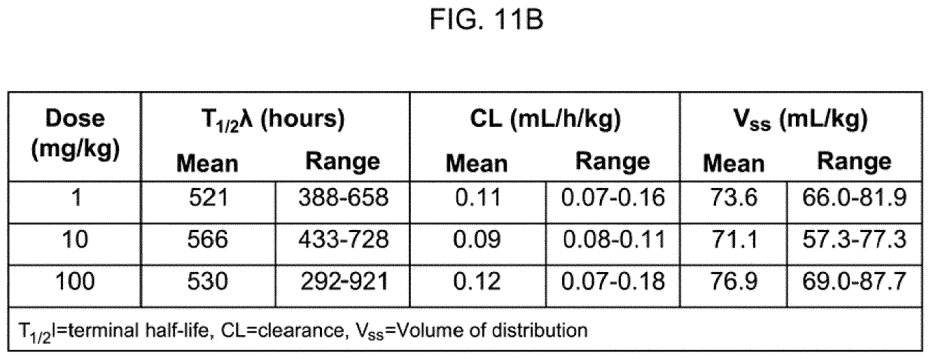

The objectives of the non-GLP exploratory study were to determine the toxicity and PK profiles of the pH-sensitive anti-VISTA antibody SNS-101 following a single intravenous infusion over a period of 1 hour and to assess the persistence, delayed onset or reversibility of any changes following a 28-day observation period in cynomolgus monkeys (the non-pH-sensitive anti-VISTA antibody h26A was included as a positive control). SNS-101 and h26A were administered to groups of monkeys (n=4; 2/sex) once via intravenous infusion, using a temporary catheter, for a period of 1 hour. The PK profile of SNS-101 was assessed in male and female cynomolgus monkeys at doses of 1, 10, and 100 mg/kg (FIG. 11A-FIG. 11B). In addition, the comparator, the non-pH-sensitive antibody h26A, was tested as well at 10 mg/kg. FIG. 11A shows the resulting PK profiles of SNS-101 and non-pH-selective antibody h26A.

H26A displayed pronounced TMDD and was below limit of quantitation at 48 hrs. In contrast, dose-proportional exposure of SNS-101 was observed following a single 1-h IV infusion of 1, 10 or 100 mg of SNS-101 per kg of body weight in the monkey. Although only 2 animals per sex were evaluated at each dose level, there were no apparent differences in PK profiles of male versus female animals. The mean concentration-time profiles and PK parameter estimates for male and female animals were similar within each dose group, therefore data for males and females were combined (FIG. 11B).

Taken together, plasma CL, T_{1/2λ} and V_{SS} of SNS-101 over the dose range of 1 to 100 mg/kg was very similar. All of these findings suggest linear disposition characteristics of SNS-101 in the monkey, which is consistent with the absence of TMDD.

### Example 6. SNS-101 Demonstrates Strong Combinatorial Activity with Anti-PD-1 Antibody and Increases CD8+ T-Cells in a MC-38 Model in Human VISTA Knock-In Mice

The therapeutic potential of SNS-101 was explored in the MC38 syngeneic tumor model in human VISTA knock-in mice. MC-38 is a colon adenocarcinoma model isolated from a colon tumor in a C57BL/6 mouse following long term exposure to the carcinogen DMH (1,2-dimethylhydrazinedihydrochloride). In order to minimize potentially neutralizing anti-SNS-101 antibodies and maximize interaction with mouse Fc receptors on immune cells, SNS-101 was grafted onto the mouse IgG2a backbone (SNS-101-m2), which is equivalent to human IgG1.

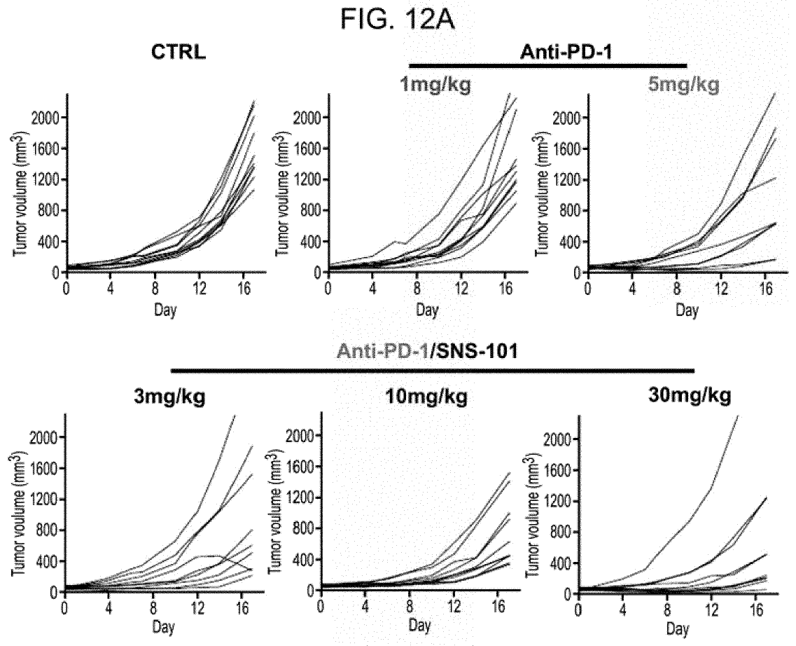

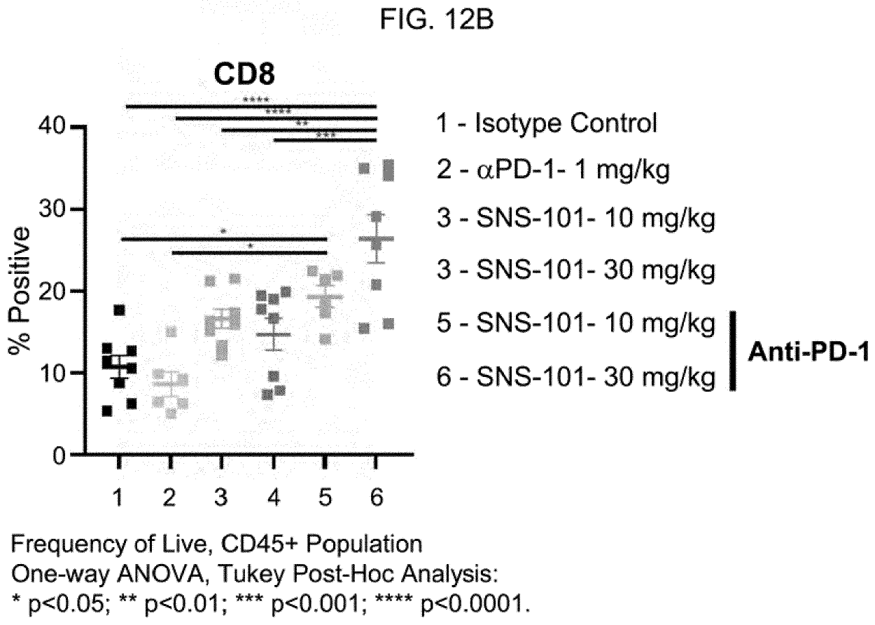

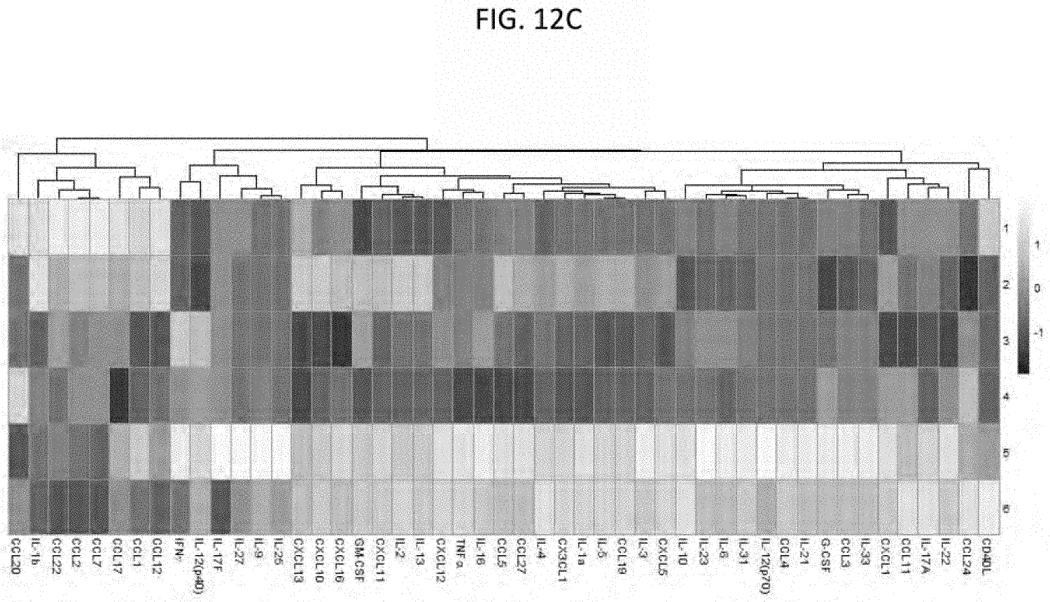

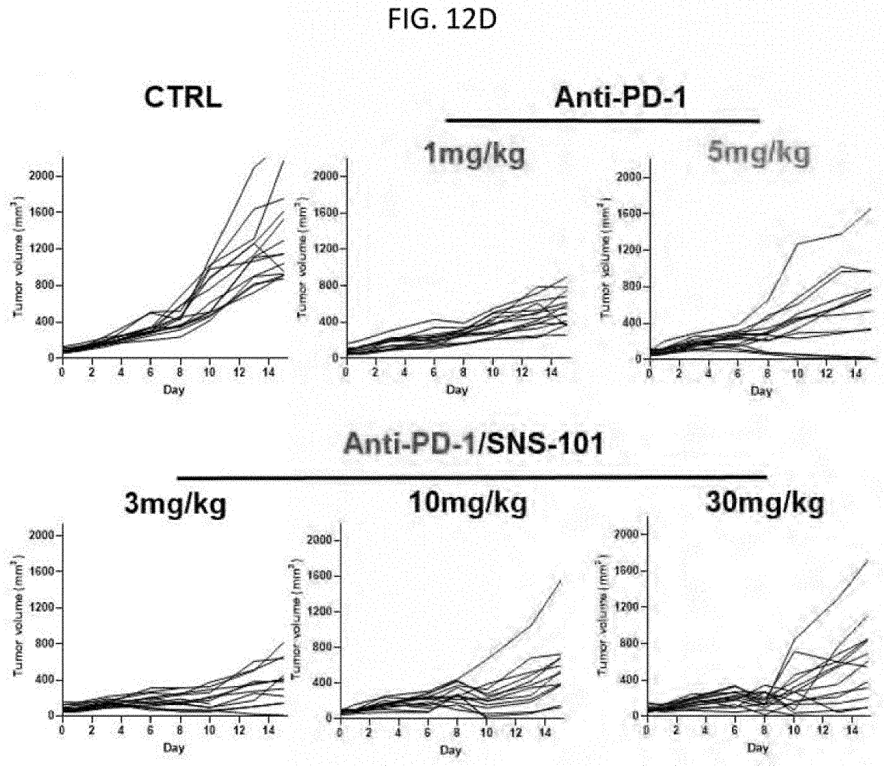

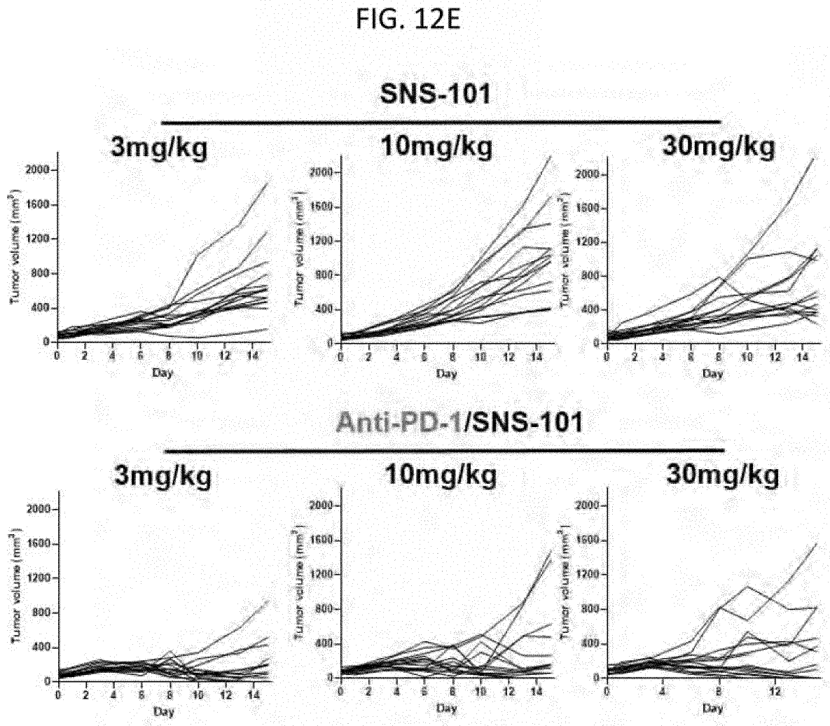

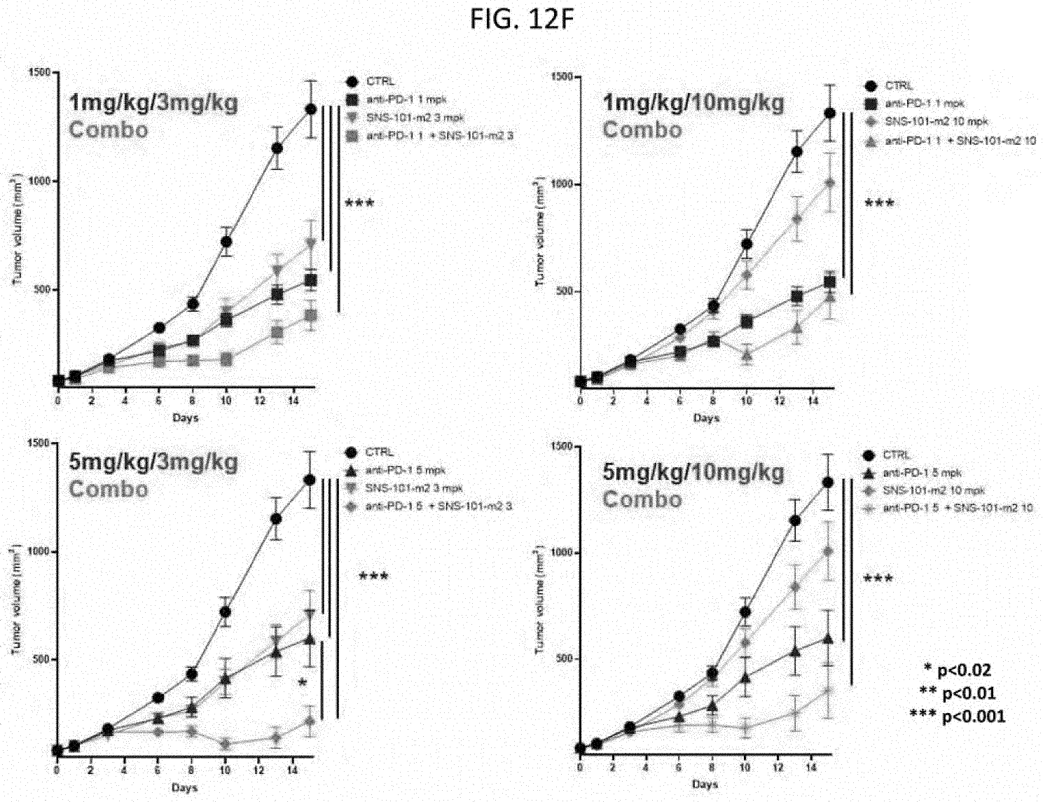

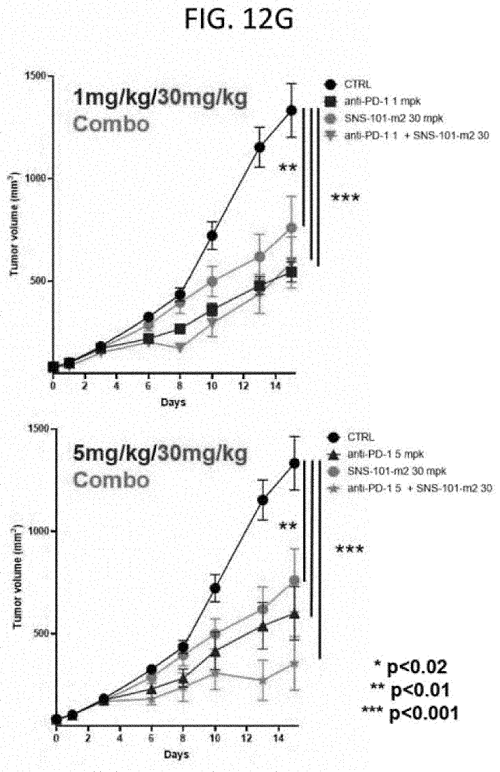

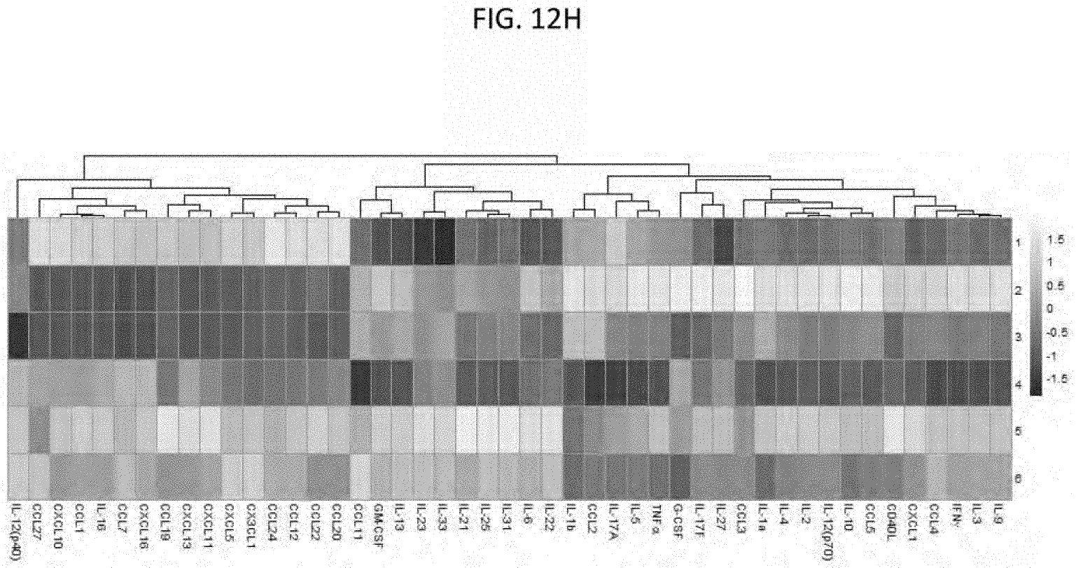

The goal of this study was to assess anti-tumor activity of anti-VISTA antibody SNS-101 in combination with an anti-mouse PD-1 antibody in established MC-38 tumors. Mice were treated with either anti-murine PD-1 antibody (RMP1-14) alone at 1 mg/kg and 5 mg/kg or in combination with SNS-101-m2 at 3 mg/kg, 10 mg/kg and 30 mg/kg respectively (FIG. 12A—FIG. 12B).

1×10⁶ MC-38 cells were implanted into female VISTA knock-in mice. Mice were randomized (n=12/cohort) once tumor volumes reached ˜0.60-80 mm³. Antibodies were administered intraperitoneally twice/week for anti-murine PD-1 antibody (RMP1-14) or three times per week for SNS-101-m2. Effects on tumor volume are shown in FIG. 12A. The groups were treated with (1) IgG CTRL mouse & rat, (2) IgG CTRL mouse & rat anti-mPD-1, or (3) rat anti-mPD-1 & anti-VISTA.

The anti-PD-1 treatment arm at 1 mg/kg did not induce statistically significant tumor growth inhibition compared to isotype control treated animals. However, the combination of 10 mg/kg and 30 mg/kg of SNS-101-m2 with anti-PD-1 at 1 mg/kg induced significant tumor growth inhibition (P-value of 0.002 and 0.004, respectively; Mann-Whitney unpaired t test).

At Day 17 post-treatment, single cell suspensions were generated from tumor extracts through physical and enzymatic dissociation. Cell concentrations were determined and 1×10⁶ cells per sample measured were subjected to viability, αCD45, and αCD8 staining in V-bottom, 96-well plates. The frequency of CD8+ cells was determined in the singlet, live, CD45+ population by analytical flow cytometry analysis (FIG. 12B). There was a significantly higher frequency of CD8+ populations in the combination of 30 mg/kg SNS-101-m2 and anti-murine PD-1 group relative to all other groups except the combination of 10 mg/kg SNS-101-m2 and anti-murine PD-1 group (p<0.05, p<0.01, p<0.001, p<0.0001).

Cytokines in plasma samples isolated from mice at Day 17 were quantitated using a Bio-Rad Bio-Plex 200 instrument, with validation procedure and instrument calibration performed as described in the instrument manual prior to running the assays. The assays used to measure analytes were: Bio-Plex Pro Mouse Cytokine 23-plex Assay (Bio-Rad cat. #M60009RDPD), Bio-Plex Pro Mouse Chemokine Panel 31-Plex Assay (Bio-Rad cat. #12009159), and Bio-Plex Pro Mouse Th17 Cytokine 10-plex Assay (Bio-Rad cat. #12010828). Assays were conducted following the kit manuals.

To visualize the data, levels of analytes were first log-transformed, and then the arithmetic mean of each treatment group was obtained. Finally, Z-scores were calculated for the group means and used to draw the heatmap (R Pheatmap package) (FIG. 12C). The dendrogram were generated using the hierarchical clustering based on the Euclidean distance between the cytokine profiles of different groups. As shown in FIG. 12C, families of cytokines were identified with both elevated as well as decreased levels in groups threated with SNS-101-m2 and anti-murine PD-1 (5,6) compared to either untreated control group (1), or a group receiving anti-murine PD-1 alone (2). For statistical analysis, the analysis of variance (ANOVA) was performed on the log-transformed cytokine data with the false discovery rate (FDR) for multiple comparison correction. The follow-up analysis was only carried out on those cytokines showing significant FDR-adjusted p-value (p_{adj}<0.05) and done with the Tukey HSD test (R package) (see Tables 34A-34C).

TABLE 34A
Statistical analysis of cytokine levels in different treatment groups

| | ANOVA | | |
|---|---|---|---|
| Cytokine | F Value | Prob | P adj |
| GM-CSF | 46.845 | 4.14E−16 | 2.52E−14 |
| IL-1a | 8.897 | 8.02E−06 | 3.45E−04 |
| IL-1b | 12.565 | 1.74E−07 | 8.55E−06 |
| IL-2 | 17.511 | 2.42E−09 | 1.35E−07 |
| IL-3 | 13.231 | 9.31E−08 | 4.66E−06 |
| IL-4 | 6.358 | 1.77E−04 | 6.19E−03 |
| IL-9 | 5.187 | 8.54E−04 | 2.39E−02 |
| IL-10 | 8.276 | 1.65E−05 | 6.59E−04 |
| IL-13 | 45.699 | 6.39E−16 | 3.84E−14 |
| CXCL2 | 5.556 | 5.14E−04 | 1.65E−02 |
| CXCL4 | 4.698 | 1.70E−03 | 4.41E−02 |
| CXCL5 | 9.840 | 2.81E−06 | 1.29E−04 |
| TNF-a | 5.148 | 9.01E−04 | 2.43E−02 |
| CXCL13 | 5.261 | 7.70E−04 | 2.36E−02 |
| CCL27 | 6.383 | 1.71E−04 | 6.16E−03 |
| CCL24 | 8.737 | 9.63E−06 | 4.05E−04 |
| CX3CL1 | 24.029 | 2.47E−11 | 1.46E−09 |
| CXCL11 | 109.087 | 6.18E−23 | 3.96E−21 |
| IL-16 | 5.261 | 7.71E−04 | 2.36E−02 |
| CXCL10 | 17.310 | 2.83E−09 | 1.56E−07 |
| CCL7 | 13.535 | 7.03E−08 | 3.58E−06 |
| CCL12 | 5.270 | 7.60E−04 | 2.36E−02 |
| CCL22 | 9.652 | 3.45E−06 | 1.52E−04 |
| CCL19 | 22.466 | 6.79E−11 | 3.87E−09 |
| CXCL16 | 14.951 | 1.99E−08 | 1.07E−06 |
| CXCL12 | 7.627 | 3.58E−05 | 1.40E−03 |
| IL-23 | 9.758 | 3.07E−06 | 1.38E−04 |
| IL-33 | 6.005 | 2.81E−04 | 9.56E−03 |

TABLE 34B
Statistical analysis of cytokine levels in different treatment groups

| | Tukey HSD Pairwise Comparison (P values) | | | | | | | | |
|---|---|---|---|---|---|---|---|---|---|
| Cytokine | Grp 2 v. 1 | Grp 3 v. 1 | Grp 4 v. 1 | Grp 5 v. 1 | Grp 6 v. 1 | Grp 3 v. 2 | Grp 4 v. 2 | Grp 5 v. 2 | Grp 6 v. 2 |
| GM-CSF | 7.02E−11 | 3.37E−06 | 9.98E−01 | 5.12E−12 | 1.07E−11 | 1.89E−02 | 2.51E−10 | 9.30E−01 | 9.86E−01 |
| IL-1a | 7.46E−01 | 9.87E−01 | 9.34E−01 | 3.90E−03 | 1.30E−02 | 3.56E−01 | 2.14E−01 | 1.28E−01 | 2.86E−01 |
| IL-1b | 1.00E+00 | 1.01E−04 | 1.89E−03 | 3.89E−04 | 6.37E−05 | 1.11E−04 | 2.06E−03 | 4.25E−04 | 6.99E−05 |
| IL-2 | 2.79E−05 | 8.55E−01 | 9.99E−01 | 1.46E−05 | 2.23E−06 | 1.10E−03 | 8.50E−05 | 1.00E+00 | 9.71E−01 |
| IL-3 | 8.50E−01 | 9.99E−01 | 9.92E−01 | 1.72E−05 | 4.61E−03 | 6.39E−01 | 5.14E−01 | 7.18E−04 | 9.42E−02 |
| IL-4 | 4.33E−01 | 9.94E−01 | 1.00E+00 | 7.22E−03 | 3.65E−03 | 7.67E−01 | 5.15E−01 | 4.47E−01 | 3.14E−01 |
| IL-9 | 1.00E+00 | 1.00E+00 | 9.95E−01 | 2.27E−03 | 7.03E−01 | 1.00E+00 | 9.95E−01 | 2.27E−03 | 7.03E−01 |
| IL-10 | 8.90E−01 | 1.00E+00 | 9.99E−01 | 1.17E−02 | 1.32E−02 | 8.54E−01 | 9.79E−01 | 4.96E−04 | 5.68E−04 |
| IL-13 | 1.07E−10 | 8.20E−02 | 7.92E−01 | 8.35E−12 | 1.45E−11 | 7.53E−07 | 5.91E−09 | 9.46E−01 | 9.83E−01 |
| CXCL2 | 4.39E−01 | 2.79E−02 | 4.99E−02 | 4.56E−03 | 3.29E−04 | 7.51E−01 | 8.68E−01 | 3.49E−01 | 6.33E−02 |
| CXCL4 | 1.00E+00 | 1.00E+00 | 1.00E+00 | 1.41E−02 | 2.29E−01 | 1.00E+00 | 1.00E+00 | 1.41E−02 | 2.29E−01 |
| CXCL5 | 1.33E−01 | 9.94E−01 | 4.11E−01 | 4.75E−03 | 1.55E−01 | 3.87E−02 | 8.07E−04 | 7.74E−01 | 1.00E+00 |
| TNF-a | 9.99E−01 | 1.00E+00 | 8.21E−01 | 5.65E−02 | 1.26E−01 | 1.00E+00 | 6.02E−01 | 1.30E−01 | 2.58E−01 |
| CXCL13 | 9.83E−01 | 8.80E−02 | 8.71E−02 | 9.96E−01 | 1.00E+00 | 1.62E−02 | 1.60E−02 | 1.00E+00 | 9.98E−01 |
| CCL27 | 9.18E−01 | 8.74E−01 | 4.42E−01 | 5.84E−02 | 5.31E−01 | 3.05E−01 | 6.87E−02 | 4.01E−01 | 9.78E−01 |
| CCL24 | 4.40E−01 | 9.28E−01 | 1.37E−01 | 2.42E−01 | 2.65E−03 | 7.38E−02 | 9.83E−04 | 2.41E−03 | 7.29E−06 |
| CX3CL1 | 2.98E−03 | 1.00E+00 | 6.81E−01 | 2.29E−06 | 2.68E−06 | 1.44E−03 | 2.92E−05 | 2.37E−01 | 2.58E−01 |
| CXCL11 | 1.11E−12 | 9.74E−01 | 9.98E−01 | 1.06E−12 | 1.06E−12 | 1.38E−12 | 1.08E−12 | 2.70E−01 | 4.19E−01 |
| IL-16 | 9.89E−01 | 8.01E−01 | 9.60E−01 | 9.94E−03 | 1.79E−01 | 9.86E−01 | 6.99E−01 | 4.91E−02 | 4.84E−01 |
| CXCL10 | 1.94E−02 | 4.96E−02 | 3.37E−01 | 1.41E−02 | 1.99E−02 | 1.81E−06 | 4.04E−05 | 1.00E+00 | 1.00E+00 |
| CCL7 | 7.28E−02 | 4.65E−04 | 8.31E−04 | 3.06E−06 | 6.13E−08 | 4.70E−01 | 5.88E−01 | 1.92E−02 | 6.02E−04 |
| CCL12 | 9.99E−01 | 4.77E−03 | 4.51E−02 | 5.35E−01 | 1.85E−02 | 1.32E−02 | 1.04E−01 | 7.60E−01 | 4.67E−02 |
| CCL22 | 9.09E−02 | 2.52E−02 | 1.49E−04 | 6.83E−04 | 1.85E−06 | 9.94E−01 | 2.30E−01 | 4.85E−01 | 9.70E−03 |
| CCL19 | 1.58E−02 | 8.61E−01 | 8.97E−01 | 5.20E−06 | 9.04E−06 | 5.57E−04 | 7.35E−04 | 1.22E−01 | 1.72E−01 |
| CXCL16 | 2.88E−01 | 2.54E−03 | 8.92E−01 | 2.48E−02 | 2.00E−01 | 2.78E−06 | 2.90E−02 | 8.67E−01 | 1.00E+00 |
| CXCL12 | 6.07E−01 | 4.95E−01 | 1.00E+00 | 2.82E−04 | 2.98E−03 | 1.00E+00 | 7.76E−01 | 2.92E−02 | 1.68E−01 |
| IL-23 | 1.00E+00 | 8.24E−01 | 1.00E+00 | 3.98E−05 | 7.48E−02 | 6.88E−01 | 1.00E+00 | 1.80E−05 | 4.20E−02 |
| IL-33 | 9.96E−01 | 9.93E−01 | 9.97E−01 | 2.35E−03 | 1.93E−01 | 8.96E−01 | 9.31E−01 | 5.34E−04 | 6.94E−02 |

TABLE 34C
Statistical analysis of cytokine levels in different treatment groups

| | Tukey HSD Pairwise Comparison (P values) | | | | | |
|---|---|---|---|---|---|---|
| Cytokine | Grp 4 v. 3 | Grp 5 v. 3 | Grp 6 v. 3 | Grp 5 v. 4 | Grp 6 v. 4 | Grp 6 v. 5 |
| GM-CSF | 1.34E−05 | 1.23E−03 | 2.99E−03 | 1.50E−11 | 3.47E−11 | 1.00E+00 |
| IL-1a | 1.00E+00 | 5.63E−04 | 2.07E−03 | 2.20E−04 | 8.35E−04 | 9.98E−01 |
| IL-1b | 9.34E−01 | 9.98E−01 | 1.00E+00 | 9.95E−01 | 8.86E−01 | 9.93E−01 |
| IL-2 | 9.64E−01 | 5.94E−04 | 9.74E−05 | 4.46E−05 | 6.89E−06 | 9.92E−01 |
| IL-3 | 1.00E+00 | 4.99E−06 | 1.51E−03 | 2.61E−06 | 8.27E−04 | 4.85E−01 |
| IL-4 | 9.98E−01 | 3.10E−02 | 1.67E−02 | 1.05E−02 | 5.37E−03 | 1.00E+00 |
| IL-9 | 9.98E−01 | 2.95E−03 | 7.55E−01 | 1.04E−02 | 9.41E−01 | 1.01E−01 |
| IL-10 | 9.98E−01 | 1.49E−02 | 1.67E−02 | 4.28E−03 | 4.85E−03 | 1.00E+00 |
| IL-13 | 6.73E−01 | 4.05E−08 | 7.93E−08 | 3.55E−10 | 6.75E−10 | 1.00E+00 |
| CXCL2 | 1.00E+00 | 9.85E−01 | 6.49E−01 | 9.46E−01 | 5.01E−01 | 9.52E−01 |
| CXCL4 | 1.00E+00 | 1.41E−02 | 2.29E−01 | 1.41E−02 | 2.29E−01 | 8.31E−01 |
| CXCL5 | 7.45E−01 | 9.49E−04 | 4.65E−02 | 1.19E−05 | 1.02E−03 | 7.32E−01 |
| TNF-a | 6.36E−01 | 1.16E−01 | 2.35E−01 | 1.99E−03 | 5.68E−03 | 9.99E−01 |
| CXCL13 | 1.00E+00 | 2.63E−02 | 4.64E−02 | 2.60E−02 | 4.58E−02 | 1.00E+00 |
| CCL27 | 9.75E−01 | 2.94E−03 | 7.29E−02 | 2.99E−04 | 1.10E−02 | 8.31E−01 |
| CCL24 | 6.19E−01 | 7.95E−01 | 3.72E−02 | 1.00E+00 | 6.47E−01 | 4.60E−01 |
| CX3CL1 | 8.19E−01 | 1.03E−06 | 1.20E−06 | 1.83E−08 | 2.14E−08 | 1.00E+00 |
| CXCL11 | 8.36E−01 | 1.06E−12 | 1.06E−12 | 1.06E−12 | 1.06E−12 | 1.00E+00 |
| IL-16 | 3.07E−01 | 2.01E−01 | 8.64E−01 | 8.59E−04 | 2.69E−02 | 8.32E−01 |
| CXCL10 | 9.32E−01 | 1.23E−06 | 1.87E−06 | 2.75E−05 | 4.17E−05 | 1.00E+00 |
| CCL7 | 1.00E+00 | 6.33E−01 | 8.78E−02 | 5.14E−01 | 5.71E−02 | 8.39E−01 |
| CCL12 | 9.59E−01 | 2.78E−01 | 9.96E−01 | 7.72E−01 | 9.99E−01 | 5.58E−01 |
| CCL22 | 5.17E−01 | 8.08E−01 | 3.95E−02 | 9.97E−01 | 7.58E−01 | 4.59E−01 |
| CCL19 | 1.00E+00 | 1.22E−07 | 2.11E−07 | 1.63E−07 | 2.83E−07 | 1.00E+00 |
| CXCL16 | 4.66E−02 | 6.86E−08 | 1.42E−06 | 1.20E−03 | 1.70E−02 | 9.38E−01 |
| CXCL12 | 6.72E−01 | 4.54E−02 | 2.35E−01 | 6.77E−04 | 6.69E−03 | 9.71E−01 |
| IL-23 | 8.56E−01 | 1.85E−03 | 6.08E−01 | 4.95E−05 | 8.72E−02 | 1.22E−01 |
| IL-33 | 1.00E+00 | 1.20E−02 | 4.82E−01 | 8.98E−03 | 4.18E−01 | 5.10E−01 |

### Example 7. SNS-101 Demonstrates Mono and Strong Combinatorial Activity with Anti-PD-1 and in MB49 Model in Human VISTA Knock-In Mice

The therapeutic potential of SNS-101 was explored in the MB49 syngeneic tumor model in human VISTA knock-in mice. MB49 cells (urothelial carcinoma) were derived from a C57BL/6 mouse following exposure of primary bladder cells to DMBA (7,12-dimethylbenz[a]anthracene) for 24 hours followed by culturing (White-Gilbertson et al., Bladder (San Franc). 2016; 3(1):e22). MB49 is considered to have a cold tumor phenotype with very low levels of CD8+ and CD4+T cell infiltration (<3%) and significant presence of immunosuppressive myeloid populations (˜65%). In order to minimize potentially neutralizing anti-SNS-101 antibodies and maximize interaction with mouse Fc receptors on immune cells, SNS-101 was grafted onto the mouse IgG2a backbone (SNS-101-m2), which is equivalent to human IgG1.

The goal of this study was to assess anti-tumor activity of anti-VISTA antibody SNS-101 alone, and in dose-response in combination with different anti-mouse PD-1 doses (similar to MC38). Mice were treated with either anti-murine PD-1 antibody (RMP1-14) alone at 1 mg/kg and 5 mg/kg or in combination with SNS-101-m2 at 3 mg/kg, 10 mg/kg and 30 mg/kg, respectively (FIG. 12D—FIG. 12G).

1×10⁶ MB49 cells were implanted into male VISTA knock-in mice. Mice were randomized (n=14/cohort) once tumor volumes reached ˜70-100 mm³. Antibodies were administered intraperitoneally twice/week for anti-murine PD-1 antibody (RMP1-14) or three times per week for SNS-101-m2. Effects on tumor volumes of individual mice (FIG. 12D, FIG. 12E) and aggregated treatment groups (FIG. 12F, FIG. 12G) are shown. The groups were treated with (1) IgG CTRL mouse & rat, (2) IgG CTRL mouse & rat anti-mPD-1, or (3) rat anti-mPD-1 & anti-VISTA.

In this in vivo study anti-VISTA SNS-101-m2 was either dosed alone (3, 10 or 30 mg/kg) or in combination with anti-mouse PD-1 (1 or 5 mg/kg) for two weeks. Antibody treatment was well tolerated, as no significant bodyweight changes were observed. Anti-mPD-1 at 1 mg/kg and 5 mg/kg resulted in substantial tumor growth inhibition (59%, P=0.0001; 55%, P=0.0003). Monotherapy of SNS-101 at 3, 10 and 30 mg/kg showed tumor growth inhibition (46%, 23%, and 43%), but only reached statistical significance at the lowest and highest doses, respectively. Combination of SNS-101-m2 at 3, 10 and 30 mg/kg with either 1 mg/kg or 5 mg/kg of anti-mouse PD-1 resulted in statistically significant tumor growth inhibition (71%, 64%, 56%, 83%, 73% and 73%, all P<0.0001) compared to the isotype control group. However, only the combination of SNS-101-m2 at 3 mg/kg with 5 mg/kg of anti-mouse PD-1 resulted in statistically significant tumor growth inhibition (83%, P<0.02) compared to anti-mPD-1 group at 5 mg/kg. Other combinations induced tumor growth inhibition, but did not reach statistical significance compared to corresponding anti-mPD-1 treatment groups

Cytokines in plasma samples isolated from mice at Day 15 were quantitated using a Bio-Rad Bio-Plex 200 instrument, with validation procedure and instrument calibration performed as described in the instrument manual prior to running the assays. The assays used to measure analytes were: Bio-Plex Pro Mouse Cytokine 23-plex Assay (Bio-Rad cat. #M60009RDPD), Bio-Plex Pro Mouse Chemokine Panel 31-Plex Assay (Bio-Rad cat. #12009159), and Bio-Plex Pro Mouse Th17 Cytokine 10-plex Assay (Bio-Rad cat. #12010828). Assays were conducted following the kit manuals.

To visualize the data, levels of analytes were first log-transformed, and then the arithmetic mean of each treatment group was obtained. Finally, Z-scores were calculated for the group means and used to draw the heatmap (R Pheatmap package) (FIG. 12H). The dendrogram were generated using the hierarchical clustering based on the Euclidean distance between the cytokine profiles of different groups. As shown in FIG. 12H, families of cytokines were identified with both elevated as well as decreased levels in groups treated with SNS-101-m2 and anti-murine PD-1 (5, 6) compared to either untreated control group (1), or a group receiving either SNS-101-m2 (4) or anti-murine PD-1 alone (2, 3). For statistical analysis, the analysis of variance (ANOVA) was performed on the log-transformed cytokine data with the false discovery rate (FDR) for multiple comparison correction. The follow-up analysis was only carried out on those cytokines showing significant FDR-adjusted p-value (p_{adj}<0.05) and done with the Tukey HSD test (R package) (see Tables 35A-35C).

TABLE 35A
Statistical analysis of cytokine levels in different treatment groups

| | ANOVA | | |
|---|---|---|---|
| Cytokine | F Value | Prob | P adj |
| CSF2 | 1.66E+01 | 4.84E−09 | 2.22E−07 |
| IL-1a | 1.01E+01 | 2.04E−06 | 9.20E−05 |
| IL-1b | 6.89E+00 | 8.94E−05 | 3.31E−03 |
| IL-2 | 7.09E+00 | 6.91E−05 | 2.62E−03 |
| IL-3 | 7.94E+00 | 2.45E−05 | 1.03E−03 |
| IL-4 | 8.71E+00 | 9.93E−06 | 4.27E−04 |
| IL-9 | 4.98E+00 | 1.15E−03 | 3.09E−02 |
| IL-10 | 5.77E+00 | 3.83E−04 | 1.26E−02 |
| IL12A | 7.84E+00 | 2.75E−05 | 1.13E−03 |
| IL-13 | 3.25E+01 | 2.13E−13 | 1.00E−11 |
| IL-17A | 6.43E+00 | 1.61E−04 | 5.80E−03 |
| CXCL1 | 7.75E+00 | 3.07E−05 | 1.23E−03 |
| CCL3 | 5.71E+00 | 4.18E−04 | 1.34E−02 |
| CCL4 | 5.28E+00 | 7.50E−04 | 2.10E−02 |
| TNF | 4.70E+00 | 1.68E−03 | 4.20E−02 |
| CCL27 | 6.29E+00 | 1.93E−04 | 6.76E−03 |
| CXCL5 | 5.31E+00 | 7.23E−04 | 2.10E−02 |
| CCL24 | 5.45E+00 | 5.94E−04 | 1.78E−02 |
| CX3CL1 | 6.13E+00 | 2.40E−04 | 8.16E−03 |
| CXCL11 | 7.71E+00 | 3.23E−05 | 1.26E−03 |
| CXCL10 | 5.62E+00 | 4.71E−04 | 1.46E−02 |
| CCL7 | 4.57E+00 | 2.04E−03 | 4.89E−02 |
| CCL19 | 8.88E+00 | 8.18E−06 | 3.60E−04 |
| CXCL16 | 4.87E+00 | 1.33E−03 | 3.45E−02 |

TABLE 35B
Statistical analysis of cytokine levels in different treatment groups

| | Tukey HSD Pairwise Comparison (P values) | | | | | | | |
|---|---|---|---|---|---|---|---|---|
| Cytokine | Grp 2 v. 1 | Grp 3 v. 1 | Grp 4 v. 1 | Grp 5 v. 1 | Grp 6 v. 1 | Grp 3 v. 2 | Grp 4 v. 2 | Grp 5 v. 2 |
| CSF2 | 3.89E−06 | 9.14E−03 | 1.00E+00 | 1.18E−06 | 1.83E−04 | 1.53E−01 | 5.35E−06 | 9.99E−01 |
| IL-1a | 1.61E−03 | 3.52E−01 | 5.75E−01 | 5.71E−02 | 9.95E−01 | 2.51E−01 | 8.55E−06 | 7.82E−01 |
| IL-1b | 2.60E−01 | 9.82E−01 | 9.26E−02 | 5.63E−01 | 4.03E−01 | 6.62E−01 | 1.92E−04 | 4.82E−03 |
| IL-2 | 1.46E−03 | 9.83E−01 | 9.98E−01 | 2.31E−02 | 8.81E−01 | 1.05E−02 | 3.67E−04 | 9.24E−01 |
| IL-3 | 9.52E−04 | 9.49E−01 | 9.34E−01 | 3.88E−02 | 3.14E−01 | 1.24E−02 | 4.84E−05 | 7.74E−01 |
| IL-4 | 6.17E−04 | 8.26E−01 | 9.74E−01 | 8.15E−03 | 9.56E−01 | 2.10E−02 | 5.72E−05 | 9.52E−01 |
| IL-9 | 1.75E−02 | 1.00E+00 | 9.62E−01 | 2.24E−01 | 6.51E−01 | 3.54E−02 | 1.68E−03 | 8.72E−01 |
| IL-10 | 5.32E−03 | 9.99E−01 | 9.81E−01 | 3.89E−01 | 1.00E+00 | 1.46E−02 | 6.51E−04 | 4.29E−01 |
| IL12A | 1.14E−03 | 9.94E−01 | 9.49E−01 | 3.72E−02 | 8.96E−01 | 5.72E−03 | 7.03E−05 | 8.14E−01 |
| IL-13 | 1.31E−08 | 1.32E−06 | 9.95E−01 | 1.34E−10 | 1.57E−08 | 7.20E−01 | 7.06E−08 | 6.92E−01 |
| IL-17A | 9.93E−01 | 2.59E−01 | 1.21E−03 | 6.90E−01 | 1.08E−01 | 8.55E−02 | 2.17E−04 | 3.52E−01 |
| CXCL1 | 2.23E−03 | 8.32E−01 | 1.00E+00 | 8.14E−04 | 4.51E−01 | 5.85E−02 | 1.91E−03 | 9.99E−01 |
| CCL3 | 1.07E−03 | 9.50E−01 | 9.99E−01 | 8.68E−01 | 9.65E−01 | 1.37E−02 | 3.07E−04 | 2.64E−02 |
| CCL4 | 2.20E−02 | 9.47E−01 | 9.60E−01 | 9.99E−02 | 2.44E−01 | 1.72E−01 | 2.11E−03 | 9.88E−01 |
| TNF | 2.06E−01 | 9.95E−01 | 3.75E−01 | 9.09E−01 | 8.88E−01 | 6.91E−02 | 1.32E−03 | 7.79E−01 |
| CCL27 | 9.03E−04 | 9.03E−04 | 3.36E−01 | 1.02E−01 | 8.63E−01 | 1.00E+00 | 1.87E−01 | 5.09E−01 |
| CXCL5 | 1.39E−02 | 1.39E−02 | 1.04E−01 | 9.62E−01 | 1.00E+00 | 1.00E+00 | 9.63E−01 | 1.04E−01 |
| CCL24 | 2.16E−03 | 2.16E−03 | 7.47E−03 | 4.56E−01 | 3.54E−01 | 1.00E+00 | 9.98E−01 | 2.17E−01 |
| CX3CL1 | 2.43E−02 | 2.43E−02 | 6.57E−02 | 1.00E+00 | 1.00E+00 | 1.00E+00 | 9.99E−01 | 4.10E−02 |
| CXCL11 | 1.14E−02 | 1.14E−02 | 4.89E−01 | 7.50E−01 | 9.46E−01 | 1.00E+00 | 4.92E−01 | 1.93E−04 |
| CXCL10 | 2.53E−03 | 2.53E−03 | 4.43E−01 | 9.68E−01 | 3.56E−01 | 1.00E+00 | 2.47E−01 | 2.30E−02 |
| CCL7 | 2.39E−02 | 2.39E−02 | 9.95E−01 | 9.93E−01 | 9.98E−01 | 1.00E+00 | 8.66E−02 | 9.21E−02 |
| CCL19 | 7.41E−03 | 7.41E−03 | 4.33E−02 | 7.40E−01 | 3.88E−01 | 1.00E+00 | 9.85E−01 | 1.11E−04 |
| CXCL16 | 2.81E−02 | 2.81E−02 | 9.99E−01 | 1.00E+00 | 9.64E−01 | 1.00E+00 | 6.73E−02 | 2.03E−02 |

TABLE 35C
Statistical analysis of cytokine levels in different treatment groups

| | Tukey HSD Pairwise Comparison (P values) | | | | | | |
|---|---|---|---|---|---|---|---|
| Cytokine | Grp 6 v. 2 | Grp 4 v. 3 | Grp 5 v. 3 | Grp 6 v. 3 | Grp 5 v. 4 | Grp 6 v. 4 | Grp 6 v. 5 |
| CSF2 | 8.42E−01 | 1.20E−02 | 7.03E−02 | 7.85E−01 | 1.63E−06 | 2.49E−04 | 6.39E−01 |
| IL-1a | 3.22E−04 | 8.60E−03 | 9.40E−01 | 1.37E−01 | 5.66E−04 | 8.69E−01 | 1.52E−02 |
| IL-1b | 2.25E−03 | 1.69E−02 | 1.96E−01 | 1.16E−01 | 8.96E−01 | 9.67E−01 | 1.00E+00 |
| IL-2 | 3.15E−02 | 8.67E−01 | 1.17E−01 | 9.98E−01 | 6.85E−03 | 6.41E−01 | 2.60E−01 |
| IL-3 | 2.09E−01 | 4.64E−01 | 2.56E−01 | 8.32E−01 | 2.96E−03 | 4.53E−02 | 9.14E−01 |
| IL-4 | 7.75E−03 | 3.79E−01 | 1.59E−01 | 9.99E−01 | 8.96E−04 | 6.04E−01 | 7.27E−02 |
| IL-9 | 4.31E−01 | 8.81E−01 | 3.54E−01 | 8.10E−01 | 3.75E−02 | 2.00E−01 | 9.73E−01 |
| IL-10 | 3.43E−03 | 8.93E−01 | 6.12E−01 | 9.95E−01 | 1.08E−01 | 9.94E−01 | 3.07E−01 |
| IL12A | 2.30E−02 | 7.10E−01 | 1.30E−01 | 9.96E−01 | 3.33E−03 | 3.95E−01 | 3.32E−01 |
| IL-13 | 1.00E+00 | 7.30E−06 | 6.57E−02 | 7.52E−01 | 6.65E−10 | 8.43E−08 | 6.58E−01 |
| IL-17A | 2.91E−02 | 2.95E−01 | 9.76E−01 | 9.98E−01 | 6.64E−02 | 5.55E−01 | 8.39E−01 |
| CXCL1 | 2.24E−01 | 8.06E−01 | 2.57E−02 | 9.87E−01 | 6.96E−04 | 4.20E−01 | 1.16E−01 |
| CCL3 | 1.12E−02 | 7.94E−01 | 1.00E+00 | 1.00E+00 | 6.49E−01 | 8.31E−01 | 1.00E+00 |
| CCL4 | 8.88E−01 | 5.27E−01 | 4.80E−01 | 7.58E−01 | 1.27E−02 | 4.10E−02 | 9.98E−01 |
| TNF | 1.73E−02 | 6.96E−01 | 6.41E−01 | 9.93E−01 | 4.94E−02 | 9.45E−01 | 3.10E−01 |
| CCL27 | 2.36E−02 | 1.87E−01 | 5.09E−01 | 2.36E−02 | 9.88E−01 | 9.43E−01 | 6.43E−01 |
| CXCL5 | 2.20E−02 | 9.63E−01 | 1.04E−01 | 2.20E−02 | 4.48E−01 | 1.50E−01 | 9.87E−01 |
| CCL24 | 2.95E−01 | 9.98E−01 | 2.17E−01 | 2.95E−01 | 4.32E−01 | 5.41E−01 | 1.00E+00 |
| CX3CL1 | 1.09E−02 | 9.99E−01 | 4.10E−02 | 1.09E−02 | 1.05E−01 | 3.17E−02 | 9.96E−01 |
| CXCL11 | 1.04E−01 | 4.92E−01 | 1.93E−04 | 1.04E−01 | 3.41E−02 | 9.47E−01 | 2.37E−01 |
| CXCL10 | 3.20E−01 | 2.47E−01 | 2.30E−02 | 3.20E−01 | 8.91E−01 | 1.00E+00 | 8.24E−01 |
| CCL7 | 6.77E−02 | 8.66E−02 | 9.21E−02 | 6.77E−02 | 1.00E+00 | 1.00E+00 | 1.00E+00 |
| CCL19 | 5.00E−01 | 9.85E−01 | 1.11E−04 | 5.00E−01 | 9.07E−04 | 8.80E−01 | 2.12E−02 |
| CXCL16 | 1.78E−01 | 6.73E−02 | 2.03E−02 | 1.78E−01 | 9.97E−01 | 9.97E−01 | 9.35E−01 |

### Example 8. Cytokine Release Profile of SNS-101 in an In Vivo Cytokine Release Syndrome Model

The cytokine release profile of SNS-101 was studied in a humanized mouse model. BRGSF-HIS mice (reconstituted functional human immune system) were generated as previously described (Lopez-Lastra et al. Blood Adv, 2017, 1(10):601-614). BRGSF-HIS mice were quality controlled (QC) by assessing circulating human CD45⁺ cells and specific immune cell subsets. Human myeloid cell development in BRGSF-HIS QC (n=60) mice was enhanced by intraperitoneal (IP) injection of hFlt3L-Fc. SNS-101 was compared to clinical stage, non-pH-selective anti-VISTA antibody JNJ (variable region JNJ-61610588 antibody cloned onto human IgG1 backbone). Serum was collected at indicated time points (hours post injection).

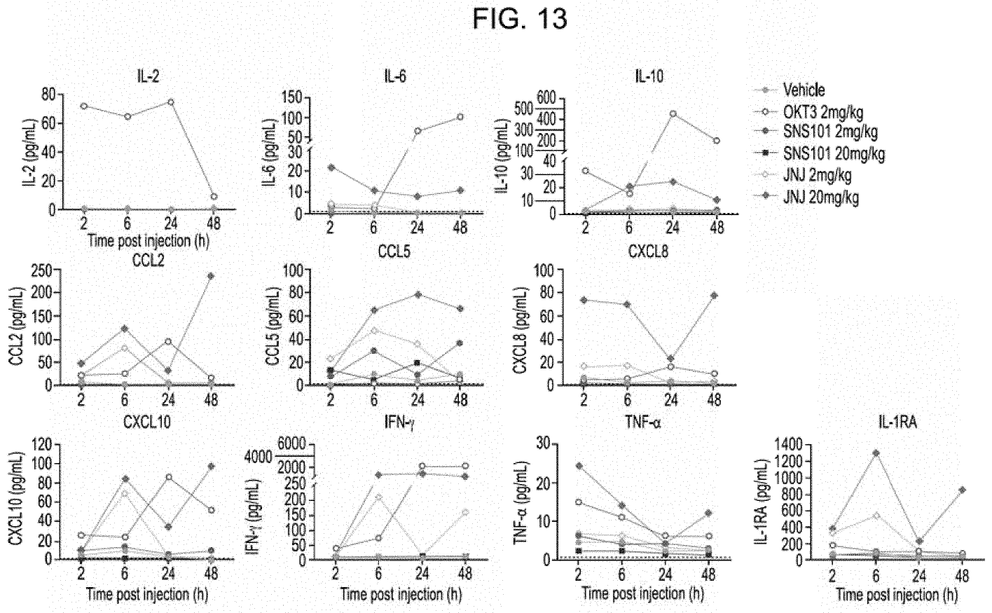

Serum of 6 mice per time point was collected, and cytokines were quantified by a Multiplex bead-based assay. Results are shown in FIG. 13. Positive control anti-CD3 antibody (OKT3) efficiently induced CRS. JNJ induced IL-6, IL-10, CCL-2, CCL-5, CXCL-8, CXCL-10, IFN-γ, TNF-α, and IL-1RA in a dose dependent-manner, whereas SNS-101 only very weakly induced CCL-5. These results suggest that SNS-101 has a significantly lower risk of inducing CRS than a non-pH-dependent anti-VISTA antibody.

### Example 9. SNS-101 Inhibits Interaction Between VISTA and Other Potential Binding Partners

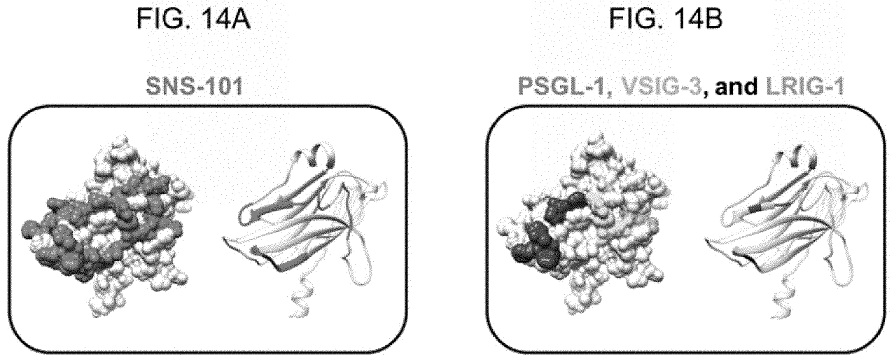

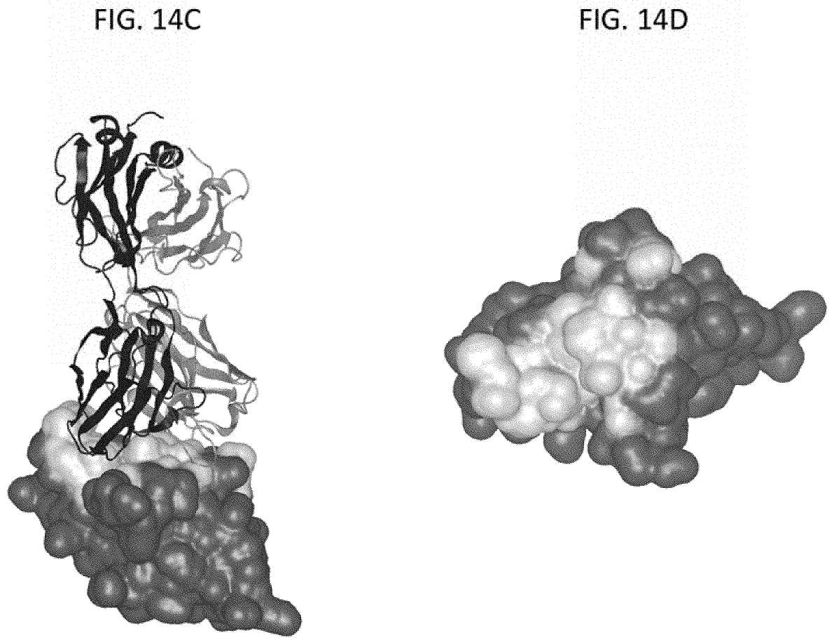

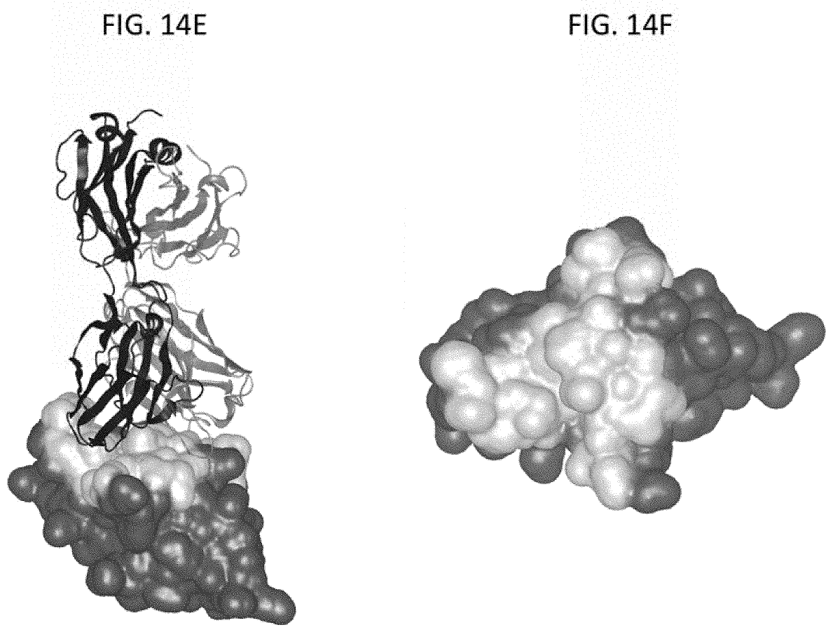

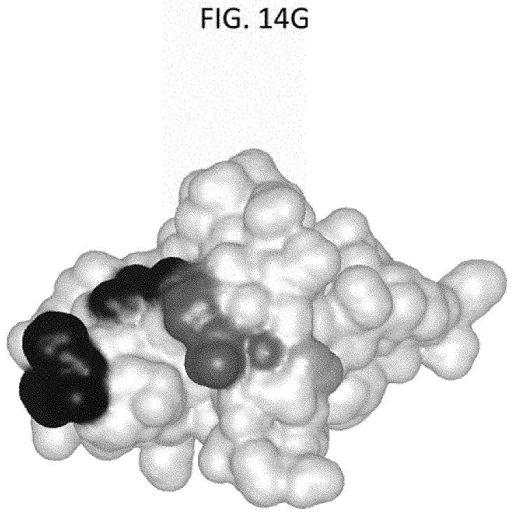

A peptide array binding analysis indicated that SNS-101 blocks the PSGL-1:VISTA protein interface. FIG. 14A and FIG. 14B show a comparison of the putative SNS-101 (gray):VISTA binding interface (FIG. 14A; defined by peptide array binding (PepScan)) to the position of 5 key histidine (His) residues involved in PSGL-1 (dark gray) binding (Johnston et al. Nature 574, 565-570 (2019)), as well as residues proposed to be involved with binding of putative partners VSIG-3 (light gray) and LRIG-1 (medium gray) (Thakkar et al. J Immnunother Cancer, 2022, 10:e003382) (FIG. 14B). The human VISTA model from AlphaFold was used.

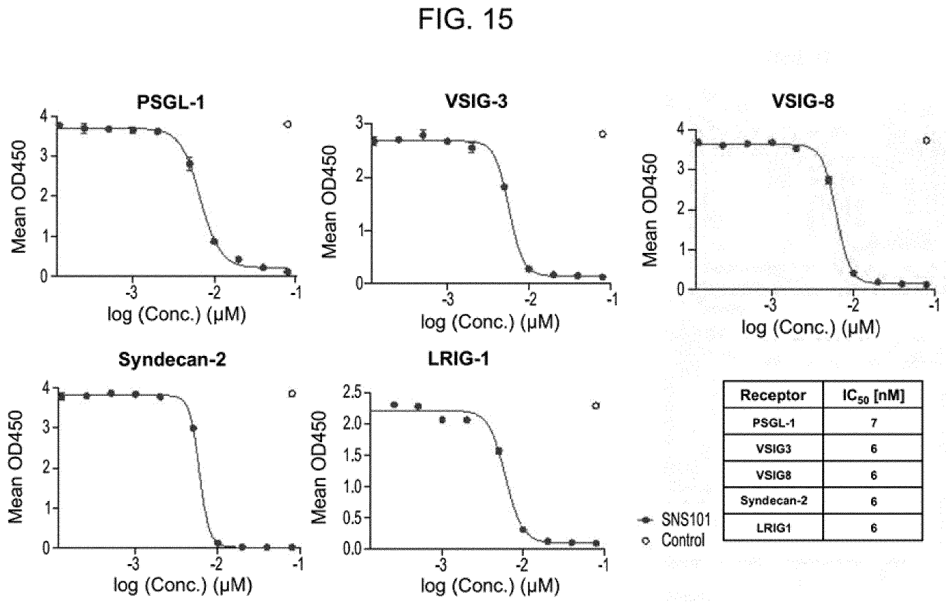

Competition experiments were carried out at pH 6.0 with dilutions of SNS-101 or isotype control pre-incubated with 1 mg/ml biotinylated hVISTA-Fc prior to addition to coated ELISA wells. Results are shown in FIG. 15. SNS-101 inhibited the interaction between VISTA and PSGL-1 and all other potential partners (VSIG-3, VSIG-8, Syndecan-2, and LRIG-1) with IC₅₀s below 7 nM.

### Example 10. Crystal Structure of hVISTA:SNS-101 Fab Complex

An X-ray crystal structure of human VISTA:human SNS-101 Fab antibody fragment complex (VISTA:Fab SNS-101 complex) was generated. Diffraction data of the VISTA:Fab SNS-101 complex were collected at the SWISS LIGHT SOURCE (SLS, Villigen, Switzerland). The structure was solved and refined with data from STARANISO with anisotropic resolution cutoffs at 2.48 Å, 2.80 Å, and 3.30 Å. The overall resolution for isotropic scaling of data is 2.97 Å (I/sigI≥_1.2 in last shell from 3.02-2.97 Å).

The structure of the VISTA:Fab SNS-101 complex at 2.59 Å resolution is shown in FIG. 14C-FIG. 14F.

hVISTA is represented in molecular surface (FIG. 14D, FIG. 14F) with SNS-101 in ribbon cartoon (FIG. 14C, FIG. 14E), respectively. The blocked surface of VISTA in the presence of SNS-101 is indicated in light gray shade whereas the unblocked portion is indicated in dark gray shade. The dark and light shade of ribbon indicate the light and heavy chain of SNS-101, respectively. The blocked surface was created with all amino acids of hVISTA that are within 4 Å (FIG. 14C, FIG. 14D) or 6A (FIG. 14E, FIG. 14F) away from any part of SNS-101.

FIG. 14G depicts PSGL-1, VSIG-3 and LRIG-1 interacting amino acid residues highlighted on the VISTA structure and a comparison between the SNS-101 blocking site and PSGL-1, VSIG-3 and LRIG-1 interacting amino acid residues.

Tables 35 and 36 provide a list of amino acid residues of hVISTA blocked in the presence of SNS-101 Fab. Underlined amino acid residues are located within 4 Å (Table 36) or 6 Å (Table 37) of any part of VISTA:SNS-101 interface. Non-underlined amino acid residues are located more than 4 Å or 6 Å away from any part of VISTA:SNS-101 interface. These residues are, however, included in the tables to indicate the amino acid stretch that may be blocked in the presence of SNS-101.

TABLE 36
hVISTA residues within 4 Å of VISTA:SNS-101 interface

| Residue number | Sequence |
|---|---|
| 69-73 | YKTWY (SEQ ID NO: 158) |
| 84-88 | SERRP (SEQ ID NO: 159) |
| 93-98 | TFQDLH (SEQ ID NO: 160) |
| 110 | S |
| 116 | R |
| 147 | L |
| 151-159 | IRHHHSEHR (SEQ ID NO: 161) |

TABLE 37
hVISTA residues within 6 Å of VISTA:SNS-101 interface

| Residue number | Sequence |
|---|---|
| 69-73 | YKTWY (SEQ ID NO: 158) |
| 80-88 | VQTCSERRP (SEQ ID NO: 162) |
| 93-98 | TFQDLH (SEQ ID NO: 160) |
| 110-117 | SHDLAQRH (SEQ ID NO: 163) |
| 147-151 | LVVEI (SEQ ID NO: 164) |
| 154-159 | HHSEHR (SEQ ID NO: 165) |

### NUMBERED EMBODIMENTS

Notwithstanding the appended claims, the disclosure sets forth the following numbered embodiments:

1. An anti-V-domain immunoglobulin suppressor of T-cell activation (VISTA) antibody or an antigen-binding portion thereof, wherein the antibody or antigen-binding portion comprises a heavy chain variable (VH) region and a light chain variable (VL) region wherein:
(a) the VH region amino acid sequence comprises a HCDR1 comprising SEQ ID NO: 54, a HCDR2 comprising SEQ ID NO: 55 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59;
(b) the VH region amino acid sequence comprises a HCDR1 comprising SEQ ID NO: 60, a HCDR2 comprising SEQ ID NO: 61 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59;
(c) the VH region amino acid sequence comprises a HCDR1 comprising SEQ ID NO: 62, a HCDR2 comprising SEQ ID NO: 63 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59;
(d) the VH region amino acid sequence comprises a HCDR1 comprising SEQ ID NO: 64, a HCDR2 comprising SEQ ID NO: 65 and a HCDR3 comprising SEQ ID NO: 66; and the VL region amino acid sequence comprises a LCDR1 comprising SEQ ID NO: 57, a LCDR2 comprising SEQ ID NO: 67 and a LCDR3 comprising SEQ ID NO: 68;
(e) the VH region amino acid sequence comprises a HCDR1 comprising SEQ ID NO: 69, a HCDR2 comprising SEQ ID NO: 70 and a HCDR3 comprising SEQ ID NO: 56; and the VL region amino acid sequence comprises a LCDR1 comprising SEQ ID NO: 57, a LCDR2 comprising SEQ ID NO: 67 and a LCDR3 comprising SEQ ID NO: 71;
(f) the VH region amino acid sequence comprises a HCDR1 comprising SEQ ID NO: 60, a HCDR2 comprising SEQ ID NO: 61 and a HCDR3 comprising SEQ ID NO: 72; and the VL region amino acid sequence comprises a LCDR1 comprising SEQ ID NO: 57, a LCDR2 comprising SEQ ID NO: 58 and a LCDR3 comprising SEQ ID NO: 68;
(g) the VH region amino acid sequence comprises a HCDR1 comprising SEQ ID NO: 73, a HCDR2 comprising SEQ ID NO: 65 and a HCDR3 comprising SEQ ID NO: 74; and the VL region amino acid sequence comprises a LCDR1 comprising SEQ ID NO: 57, a LCDR2 comprising SEQ ID NO: 58 and a LCDR3 comprising SEQ ID NO: 71;
(h) the VH region amino acid sequence comprises a HCDR1 comprising SEQ ID NO: 64, a HCDR2 comprising SEQ ID NO: 75 and a HCDR3 comprising SEQ ID NO: 66; and the VL region amino acid sequence comprises a LCDR1 comprising SEQ ID NO: 57, a LCDR2 comprising SEQ ID NO: 76 and a LCDR3 comprising SEQ ID NO: 68;
(i) the VH region amino acid sequence comprises a HCDR1 comprising SEQ ID NO: 64, a HCDR2 comprising SEQ ID NO: 65 and a HCDR3 comprising SEQ ID NO: 77; and the VL region amino acid sequence comprises a LCDR1 comprising SEQ ID NO: 57, a LCDR2 comprising SEQ ID NO: 78 and a LCDR3 comprising SEQ ID NO: 68;
(j) the VH region amino acid sequence comprises a HCDR1 comprising SEQ ID NO: 60, a HCDR2 comprising SEQ ID NO: 75 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 68;
(k) the VH region amino acid sequence comprises a HCDR1 comprising SEQ ID NO: 54, a HCDR2 comprising SEQ ID NO: 65 and a HCDR3 comprising SEQ ID NO: 79; and the VL region amino acid sequence comprises a LCDR1 comprising SEQ ID NO: 80, a LCDR2 comprising SEQ ID NO: 58 and a LCDR3 comprising SEQ ID NO: 68;
(1) the VH region amino acid sequence comprises a HCDR1 comprising SEQ ID NO: 81, a HCDR2 comprising SEQ ID NO: 82 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86;
(m) the VH region amino acid sequence comprises a HCDR1 comprising SEQ ID NO: 87, a HCDR2 comprising SEQ ID NO: 88 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86;
(n) the VH region amino acid sequence comprises a HCDR1 comprising SEQ ID NO: 89, a HCDR2 comprising SEQ ID NO: 90 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86;
(o) the VH region amino acid sequence comprises a HCDR1 comprising SEQ ID NO: 91, a HCDR2 comprising SEQ ID NO: 92 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86;
(p) the VH region amino acid sequence comprises a HCDR1 comprising SEQ ID NO: 93, a HCDR2 comprising SEQ ID NO: 94 and a HCDR3 comprising SEQ ID NO: 95; and the VL region amino acid sequence comprises a LCDR1 comprising SEQ ID NO: 96, a LCDR2 comprising SEQ ID NO: 85 and a LCDR3 comprising SEQ ID NO: 97;
(q) the VH region amino acid sequence comprises a HCDR1 comprising SEQ ID NO: 98, a HCDR2 comprising SEQ ID NO: 99 and a HCDR3 comprising SEQ ID NO: 100; and the VL region amino acid sequence comprises a LCDR1 comprising SEQ ID NO: 96, a LCDR2 comprising SEQ ID NO: 101 and a LCDR3 comprising SEQ ID NO: 102;
(r) the VH region amino acid sequence comprises a HCDR1 comprising SEQ ID NO: 103, a HCDR2 comprising SEQ ID NO: 104 and a HCDR3 comprising SEQ ID NO: 105; and the VL region amino acid sequence comprises a LCDR1 comprising SEQ ID NO: 96, a LCDR2 comprising SEQ ID NO: 106 and a LCDR3 comprising SEQ ID NO: 107;
(s) the VH region amino acid sequence comprises a HCDR1 comprising SEQ ID NO: 108, a HCDR2 comprising SEQ ID NO: 94 and a HCDR3 comprising SEQ ID NO: 105; and the VL region amino acid sequence comprises a LCDR1 comprising SEQ ID NO: 109, a LCDR2 comprising SEQ ID NO: 85 and a LCDR3 comprising SEQ ID NO: 110;
(t) the VH region amino acid sequence comprises a HCDR1 comprising SEQ ID NO: 111, a HCDR2 comprising SEQ ID NO: 94 and a HCDR3 comprising SEQ ID NO: 112; and the VL region amino acid sequence comprises a LCDR1 comprising SEQ ID NO: 96, a LCDR2 comprising SEQ ID NO: 85 and a LCDR3 comprising SEQ ID NO: 113;
(u) the VH region amino acid sequence comprises a HCDR1 comprising SEQ ID NO: 87, a HCDR2 comprising SEQ ID NO: 94 and a HCDR3 comprising SEQ ID NO: 114; and the VL region amino acid sequence comprises a LCDR1 comprising SEQ ID NO: 115, a LCDR2 comprising SEQ ID NO: 85 and a LCDR3 comprising SEQ ID NO: 116;
(v) the VH region amino acid sequence comprises a HCDR1 comprising SEQ ID NO: 93, a HCDR2 comprising SEQ ID NO: 90 and a HCDR3 comprising SEQ ID NO: 117; and the VL region amino acid sequence comprises a LCDR1 comprising SEQ ID NO: 118, a LCDR2 comprising SEQ ID NO: 85 and a LCDR3 comprising SEQ ID NO: 86;
(w) the VH region amino acid sequence comprises a HCDR1 comprising SEQ ID NO: 119, a HCDR2 comprising SEQ ID NO: 120 and a HCDR3 comprising SEQ ID NO: 121; and the VL region amino acid sequence comprises a LCDR1 comprising SEQ ID NO: 57, a LCDR2 comprising SEQ ID NO: 58 and a LCDR3 comprising SEQ ID NO: 122;
(x) the VH region amino acid sequence comprises a HCDR1 comprising SEQ ID NO: 123, a HCDR2 comprising SEQ ID NO: 124 and a HCDR3 comprising SEQ ID NO: 125; and the VL region amino acid sequence comprises a LCDR1 comprising SEQ ID NO: 126, a LCDR2 comprising SEQ ID NO: 127 and a LCDR3 comprising SEQ ID NO: 128;
(y) the VH region amino acid sequence comprises a HCDR1 comprising SEQ ID NO: 123, a HCDR2 comprising SEQ ID NO: 124 and a HCDR3 comprising SEQ ID NO: 129; and the VL region amino acid sequence comprises a LCDR1 comprising SEQ ID NO: 57, a LCDR2 comprising SEQ ID NO: 58 and a LCDR3 comprising SEQ ID NO: 130;
(z) the VH region amino acid sequence comprises a HCDR1 comprising SEQ ID NO: 131, a HCDR2 comprising SEQ ID NO: 132 and a HCDR3 comprising SEQ ID NO: 133; and the VL region amino acid sequence comprises a LCDR1 comprising SEQ ID NO: 126, a LCDR2 comprising SEQ ID NO: 134 and a LCDR3 comprising SEQ ID NO: 135;
(aa) the VH region amino acid sequence comprises a HCDR1 comprising SEQ ID NO: 131, a HCDR2 comprising SEQ ID NO: 132 and a HCDR3 comprising SEQ ID NO: 136; and the VL region amino acid sequence comprises a LCDR1 comprising SEQ ID NO: 57, a LCDR2 comprising SEQ ID NO: 58 and a LCDR3 comprising SEQ ID NO: 137;
(bb) the VH region amino acid sequence comprises a HCDR1 comprising SEQ ID NO: 54, a HCDR2 comprising SEQ ID NO: 138 and a HCDR3 comprising SEQ ID NO: 139; and the VL region amino acid sequence comprises a LCDR1 comprising SEQ ID NO: 57, a LCDR2 comprising SEQ ID NO: 58 and a LCDR3 comprising SEQ ID NO: 140; or
(cc) the VH region amino acid sequence comprises a HCDR1 comprising SEQ ID NO: 81, a HCDR2 comprising SEQ ID NO: 82 and a HCDR3 comprising SEQ ID NO: 141; and the VL region amino acid sequence comprises a LCDR1 comprising SEQ ID NO: 142, a LCDR2 comprising SEQ ID NO: 143 and a LCDR3 comprising SEQ ID NO: 144.

2. The antibody or antigen-binding portion of embodiment 1, wherein
(a) the VH region amino acid sequence comprises SEQ ID NO: 1 and the VL region amino acid sequence comprises SEQ ID NO: 2;
(b) the VH region amino acid sequence comprises SEQ ID NO: 3 and the VL region amino acid sequence comprises SEQ ID NO: 2;
(c) the VH region amino acid sequence comprises SEQ ID NO: 4 and the VL region amino acid sequence comprises SEQ ID NO: 2;
(d) the VH region amino acid sequence comprises SEQ ID NO: 5 and the VL region amino acid sequence comprises SEQ ID NO: 6;
(e) the VH region amino acid sequence comprises SEQ ID NO: 7 and the VL region amino acid sequence comprises SEQ ID NO: 8;
(f) the VH region amino acid sequence comprises SEQ ID NO: 9 and the VL region amino acid sequence comprises SEQ ID NO: 10;
(g) the VH region amino acid sequence comprises SEQ ID NO: 11 and the VL region amino acid sequence comprises SEQ ID NO: 12;
(h) the VH region amino acid sequence comprises SEQ ID NO: 13 and the VL region amino acid sequence comprises SEQ ID NO: 14;
(i) the VH region amino acid sequence comprises SEQ ID NO: 15 and the VL region amino acid sequence comprises SEQ ID NO: 16;
(j) the VH region amino acid sequence comprises SEQ ID NO: 17 and the VL region amino acid sequence comprises SEQ ID NO: 18;
(k) the VH region amino acid sequence comprises SEQ ID NO: 19 and the VL region amino acid sequence comprises SEQ ID NO: 20;
(1) the VH region amino acid sequence comprises SEQ ID NO: 21 and the VL region amino acid sequence comprises SEQ ID NO: 22;
(m) the VH region amino acid sequence comprises SEQ ID NO: 23 and the VL region amino acid sequence comprises SEQ ID NO: 22;
(n) the VH region amino acid sequence comprises SEQ ID NO: 24 and the VL region amino acid sequence comprises SEQ ID NO: 22;
(o) the VH region amino acid sequence comprises SEQ ID NO: 25 and the VL region amino acid sequence comprises SEQ ID NO: 22;
(p) the VH region amino acid sequence comprises SEQ ID NO: 26 and the VL region amino acid sequence comprises SEQ ID NO: 27;
(q) the VH region amino acid sequence comprises SEQ ID NO: 28 and the VL region amino acid sequence comprises SEQ ID NO: 29;
(r) the VH region amino acid sequence comprises SEQ ID NO: 30 and the VL region amino acid sequence comprises SEQ ID NO: 31;
(s) the VH region amino acid sequence comprises SEQ ID NO: 32 and the VL region amino acid sequence comprises SEQ ID NO: 33;
(t) the VH region amino acid sequence comprises SEQ ID NO: 34 and the VL region amino acid sequence comprises SEQ ID NO: 35;
(u) the VH region amino acid sequence comprises SEQ ID NO: 36 and the VL region amino acid sequence comprises SEQ ID NO: 37;
(v) the VH region amino acid sequence comprises SEQ ID NO: 38 and the VL region amino acid sequence comprises SEQ ID NO: 39;
(w) the VH region amino acid sequence comprises SEQ ID NO: 40 and the VL region amino acid sequence comprises SEQ ID NO: 41;
(x) the VH region amino acid sequence comprises SEQ ID NO: 42 and the VL region amino acid sequence comprises SEQ ID NO: 43;
(y) the VH region amino acid sequence comprises SEQ ID NO: 44 and the VL region amino acid sequence comprises SEQ ID NO: 45;
(z) the VH region amino acid sequence comprises SEQ ID NO: 46 and the VL region amino acid sequence comprises SEQ ID NO: 47;
(aa) the VH region amino acid sequence comprises SEQ ID NO: 48 and the VL region amino acid sequence comprises SEQ ID NO: 49;
(bb) the VH region amino acid sequence comprises SEQ ID NO: 50 and the VL region amino acid sequence comprises SEQ ID NO: 51; or
(cc) the VH region amino acid sequence comprises SEQ ID NO: 52 and the VL region amino acid sequence comprises SEQ ID NO: 53.

3. The antibody or antigen-binding portion of embodiment 1 or 2, wherein the antibody or antigen-binding portion is human or chimeric.

4. The antibody or antigen-binding portion of any one of embodiments 1-3, wherein the antibody or antigen-binding portion comprises an immunoglobulin constant region.

5. The antibody or antigen-binding portion of embodiment 4, wherein the immunoglobulin constant region is IgG, IgE, IgM, IgD, IgA or IgY.

6. The antibody or antigen-binding portion of embodiment 5, wherein the immunoglobulin constant region is IgG1, IgG2, IgG3, IgG4, IgA1 or IgA2.

7. The antibody or antigen-binding portion of embodiment 4, wherein the immunoglobulin constant region is immunologically inert.

8. The antibody or antigen-binding portion of embodiment 4, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A, L235A and G237A or a wild-type human IgG2 constant region, wherein numbering is according to the EU index as in Kabat.

9. The antibody or antigen-binding portion of embodiment 4, wherein the immunoglobulin constant region comprises any one of SEQ ID NOs: 145-149.

10. The antibody or antigen-binding portion of any one of embodiments 1-9, wherein the antibody or antigen-binding portion is an Fab, an Fab′, an F(ab′)2, an Fv, an scFv, a maxibody, a minibody, a diabody, a triabody, a tetrabody, or a bis-scFv.

11. The antibody or antigen-binding portion of any one of embodiments 1-10, wherein the antibody is monoclonal.

12. The antibody or antigen-binding portion of any one of embodiments 1-11, wherein the antibody or antigen-binding portion is tetrameric, tetravalent or multispecific.

13. The antibody or antigen-binding portion of any one of embodiments 1-12, wherein the antibody or antigen-binding portion is a bispecific antibody or antigen-binding portion that binds specifically to a first antigen and a second antigen, wherein the first antigen is VISTA and the second antigen is not VISTA.

14. An immunoconjugate comprising the antibody or antigen-binding portion of any one of embodiments 1-13, linked to a therapeutic agent.

15. The immunoconjugate of embodiment 14, wherein the therapeutic agent is a cytotoxin, a radioisotope, a chemotherapeutic agent, an immunomodulatory agent, a cytostatic enzyme, a cytolytic enzyme, a therapeutic peptide, a therapeutic nucleic acid, an anti-angiogenic agent, an anti-proliferative agent, or a pro-apoptotic agent.

16. A pharmaceutical composition comprising the antibody or antigen-binding portion of any one of embodiments 1-13 or the immunoconjugate of embodiment 14 or 15, and a pharmaceutically acceptable carrier, diluent or excipient.

17. A nucleic acid molecule encoding
(a) the VH region amino acid sequence;
(b) the VL region amino acid sequence; or
(c) both the VH and the VL region amino acid sequences of the antibody or antigen-binding portion of any one of embodiments 1-13.

18. An expression vector comprising the nucleic acid molecule of embodiment 17.

19. A recombinant host cell comprising the nucleic acid molecule of embodiment 17 or the expression vector of embodiment 18.

20. A method of producing an anti-VISTA antibody or an antigen-binding portion thereof, the method comprising:

culturing a recombinant host cell comprising the expression vector of embodiment 18 under conditions whereby the nucleic acid molecule is expressed, thereby producing the antibody or antigen-binding portion; and isolating the antibody or antigen-binding portion from the host cell or culture.

21. A method for inducing an immune response in a subject, comprising administering to the subject a therapeutically effective amount of the antibody or antigen-binding portion of any one of embodiments 1-13, the immunoconjugate of embodiment 14 or 15 or the pharmaceutical composition of embodiment 16.

22. The method of embodiment 21, wherein the immune response is an immune response against cancer cells.

23. The method of embodiment 21 or 22, wherein the immune response is antibody-dependent cellular cytotoxicity, complement-dependent cytotoxicity or antibody-dependent cellular phagocytosis.

24. A method for treating or preventing a cancer in a subject, comprising administering to the subject a therapeutically effective amount of the antibody or antigen-binding portion of any one of embodiments 1-13, the immunoconjugate of embodiment 14 or 15 or the pharmaceutical composition of embodiment 16.

25. The method of embodiment 24, wherein the cancer is a hematologic malignancy or an epithelial cancer.

26. The method of embodiment 24, wherein the cancer is squamous cell carcinoma of head and neck, melanoma, non-small cell lung cancer or colon cancer.

27. The method of embodiment 24, wherein the cancer is acute myeloid leukemia (AML).

28. The method of any one of embodiments 21-27, wherein the subject overexpresses VISTA.

29. The method of any one of embodiments 21-27, wherein the subject overexpresses VISTA on immune cells.

30. The method of embodiment 29, wherein the immune cells are tumor-infiltrating immune cells.

31. The method of embodiment 29 or 30, wherein the immune cells are myeloid cells or monocytes.

32. The method of any one of embodiments 21-31, further comprising administering to the subject an inhibitor of PD-1 or an inhibitor of PD-L1.

33. The method of embodiment 32, wherein the inhibitor of PD-1 is an anti-PD-1 antibody or antigen-binding portion thereof.

34. The method of embodiment 32, wherein the inhibitor of PD-L1 is an anti-PD-L1 antibody or antigen-binding portion thereof.

35. The antibody or antigen-binding portion of any one of embodiments 1-13, the immunoconjugate of embodiment 14 or 15 or the pharmaceutical composition of embodiment 16, for use as a medicament.

36. A method for treating a tumor in a subject, the method comprising:
(a) obtaining a VISTA:PSGL-1 proximity score for a tumor sample from the subject; wherein the VISTA:PSGL-1 proximity score is been assigned to the tumor sample by a process which comprises:
(i) obtaining an image of tissue that has been removed from the tumor sample and immunohistochemically stained for VISTA and PSGL-1 expression in a manner that allows stained VISTA cells to be distinguished from stained PSGL-1 cells;
(ii) defining in the image one or more regions of interest (ROIs) that comprises neoplastic cells and associated stroma, wherein substantially all of the neoplastic cells and associated stroma in the image are contained in the defined ROIs;
(iii) randomly creating across each defined ROI a plurality of subregions of substantially the same shape and size, wherein each of the subregions defines an area that is large enough to include a spatially proximal pair of a stained VISTA cell and a stained PSGL-1 cell and small enough to exclude pairs of stained VISTA and PSGL-1 cells that are not spatially proximal; and
(iv) calculating the percent of all of the subregions that are positive for both stained VISTA cells and stained PSGL-1 cells to generate the VISTA:PSGL-1 proximity score for the tumor sample,
(b) comparing the VISTA:PSGL-1 proximity score for the tumor sample with a threshold VISTA:PSGL-1 proximity score,
(c) classifying the tumor as biomarker positive or biomarker negative, wherein if the proximity score for the tumor sample is equal to or greater than the threshold proximity score, then the tumor is classified as positive for VISTA:PSGL-1 proximity biomarker, and if the obtained score is less than the threshold score, then the tumor is classified as negative for the VISTA:PSGL-1 proximity biomarker, and
(d) administering to the subject a therapeutically effective amount of the antibody or antigen-binding portion of any one of embodiments 1-13, the immunoconjugate of embodiment 14 or 15 or the pharmaceutical composition of embodiment 16 if the tumor is positive for the VISTA:PSGL-1 proximity biomarker.

1. An anti-V-domain immunoglobulin suppressor of T-cell activation (VISTA) antibody or an antigen-binding portion thereof, wherein the antibody or antigen-binding portion comprises a heavy chain variable (VH) region and a light chain variable (VL) region wherein:
(a) the VH region amino acid sequence comprises a HCDR1 comprising SEQ ID NO: 54, a HCDR2 comprising SEQ ID NO: 55 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59;
(b) the VH region amino acid sequence comprises a HCDR1 comprising SEQ ID NO: 60, a HCDR2 comprising SEQ ID NO: 61 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59;
(c) the VH region amino acid sequence comprises a HCDR1 comprising SEQ ID NO: 62, a HCDR2 comprising SEQ ID NO: 63 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 59;
(d) the VH region amino acid sequence comprises a HCDR1 comprising SEQ ID NO: 64, a HCDR2 comprising SEQ ID NO: 65 and a HCDR3 comprising SEQ ID NO: 66; and the VL region amino acid sequence comprises a LCDR1 comprising SEQ ID NO: 57, a LCDR2 comprising SEQ ID NO: 67 and a LCDR3 comprising SEQ ID NO: 68;
(e) the VH region amino acid sequence comprises a HCDR1 comprising SEQ ID NO: 69, a HCDR2 comprising SEQ ID NO: 70 and a HCDR3 comprising SEQ ID NO: 56; and the VL region amino acid sequence comprises a LCDR1 comprising SEQ ID NO: 57, a LCDR2 comprising SEQ ID NO: 67 and a LCDR3 comprising SEQ ID NO: 71;
(f) the VH region amino acid sequence comprises a HCDR1 comprising SEQ ID NO: 60, a HCDR2 comprising SEQ ID NO: 61 and a HCDR3 comprising SEQ ID NO: 72; and the VL region amino acid sequence comprises a LCDR1 comprising SEQ ID NO: 57, a LCDR2 comprising SEQ ID NO: 58 and a LCDR3 comprising SEQ ID NO: 68;
(g) the VH region amino acid sequence comprises a HCDR1 comprising SEQ ID NO: 73, a HCDR2 comprising SEQ ID NO: 65 and a HCDR3 comprising SEQ ID NO: 74; and the VL region amino acid sequence comprises a LCDR1 comprising SEQ ID NO: 57, a LCDR2 comprising SEQ ID NO: 58 and a LCDR3 comprising SEQ ID NO: 71;
(h) the VH region amino acid sequence comprises a HCDR1 comprising SEQ ID NO: 64, a HCDR2 comprising SEQ ID NO: 75 and a HCDR3 comprising SEQ ID NO: 66; and the VL region amino acid sequence comprises a LCDR1 comprising SEQ ID NO: 57, a LCDR2 comprising SEQ ID NO: 76 and a LCDR3 comprising SEQ ID NO: 68;
(i) the VH region amino acid sequence comprises a HCDR1 comprising SEQ ID NO: 64, a HCDR2 comprising SEQ ID NO: 65 and a HCDR3 comprising SEQ ID NO: 77; and the VL region amino acid sequence comprises a LCDR1 comprising SEQ ID NO: 57, a LCDR2 comprising SEQ ID NO: 78 and a LCDR3 comprising SEQ ID NO: 68;
(j) the VH region amino acid sequence comprises a HCDR1 comprising SEQ ID NO: 60, a HCDR2 comprising SEQ ID NO: 75 and a HCDR3 comprising SEQ ID NO: 56; and the VL region amino acid sequence comprises a LCDR1 comprising SEQ ID NO: 57, a LCDR2 comprising SEQ ID NO: 58 and a LCDR3 comprising SEQ ID NO: 68;
(k) the VH region amino acid sequence comprises a HCDR1 comprising SEQ ID NO: 54, a HCDR2 comprising SEQ ID NO: 65 and a HCDR3 comprising SEQ ID NO: 79; and the VL region amino acid sequence comprises a LCDR1 comprising SEQ ID NO: 80, a LCDR2 comprising SEQ ID NO: 58 and a LCDR3 comprising SEQ ID NO: 68;
(l) the VH region amino acid sequence comprises a HCDR1 comprising SEQ ID NO: 81, a HCDR2 comprising SEQ ID NO: 82 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86;
(m) the VH region amino acid sequence comprises a HCDR1 comprising SEQ ID NO: 87, a HCDR2 comprising SEQ ID NO: 88 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86;
(n) the VH region amino acid sequence comprises a HCDR1 comprising SEQ ID NO: 89, a HCDR2 comprising SEQ ID NO: 90 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86;
(o) the VH region amino acid sequence comprises a HCDR1 comprising SEQ ID NO: 91, a HCDR2 comprising SEQ ID NO: 92 and a HCDR3 comprising SEQ ID NO: 83; and the VL region amino acid sequence comprises a LCDR1 comprising SEQ ID NO: 84, a LCDR2 comprising SEQ ID NO: 85 and a LCDR3 comprising SEQ ID NO: 86;
(p) the VH region amino acid sequence comprises a HCDR1 comprising SEQ ID NO: 93, a HCDR2 comprising SEQ ID NO: 94 and a HCDR3 comprising SEQ ID NO: 95; and the VL region amino acid sequence comprises a LCDR1 comprising SEQ ID NO: 96, a LCDR2 comprising SEQ ID NO: 85 and a LCDR3 comprising SEQ ID NO: 97;
(q) the VH region amino acid sequence comprises a HCDR1 comprising SEQ ID NO: 98, a HCDR2 comprising SEQ ID NO: 99 and a HCDR3 comprising SEQ ID NO: 100; and the VL region amino acid sequence comprises a LCDR1 comprising SEQ ID NO: 96, a LCDR2 comprising SEQ ID NO: 101 and a LCDR3 comprising SEQ ID NO: 102;
(r) the VH region amino acid sequence comprises a HCDR1 comprising SEQ ID NO: 103, a HCDR2 comprising SEQ ID NO: 104 and a HCDR3 comprising SEQ ID NO: 105; and the VL region amino acid sequence comprises a LCDR1 comprising SEQ ID NO: 96, a LCDR2 comprising SEQ ID NO: 106 and a LCDR3 comprising SEQ ID NO: 107;
(s) the VH region amino acid sequence comprises a HCDR1 comprising SEQ ID NO: 108, a HCDR2 comprising SEQ ID NO: 94 and a HCDR3 comprising SEQ ID NO: 105; and the VL region amino acid sequence comprises a LCDR1 comprising SEQ ID NO: 109, a LCDR2 comprising SEQ ID NO: 85 and a LCDR3 comprising SEQ ID NO: 110;
(t) the VH region amino acid sequence comprises a HCDR1 comprising SEQ ID NO: 111, a HCDR2 comprising SEQ ID NO: 94 and a HCDR3 comprising SEQ ID NO: 112; and the VL region amino acid sequence comprises a LCDR1 comprising SEQ ID NO: 96, a LCDR2 comprising SEQ ID NO: 85 and a LCDR3 comprising SEQ ID NO: 113;
(u) the VH region amino acid sequence comprises a HCDR1 comprising SEQ ID NO: 87, a HCDR2 comprising SEQ ID NO: 94 and a HCDR3 comprising SEQ ID NO: 114; and the VL region amino acid sequence comprises a LCDR1 comprising SEQ ID NO: 115, a LCDR2 comprising SEQ ID NO: 85 and a LCDR3 comprising SEQ ID NO: 116;
(v) the VH region amino acid sequence comprises a HCDR1 comprising SEQ ID NO: 93, a HCDR2 comprising SEQ ID NO: 90 and a HCDR3 comprising SEQ ID NO: 117; and the VL region amino acid sequence comprises a LCDR1 comprising SEQ ID NO: 118, a LCDR2 comprising SEQ ID NO: 85 and a LCDR3 comprising SEQ ID NO: 86;
(w) the VH region amino acid sequence comprises a HCDR1 comprising SEQ ID NO: 119, a HCDR2 comprising SEQ ID NO: 120 and a HCDR3 comprising SEQ ID NO: 121; and the VL region amino acid sequence comprises a LCDR1 comprising SEQ ID NO: 57, a LCDR2 comprising SEQ ID NO: 58 and a LCDR3 comprising SEQ ID NO: 122;
(x) the VH region amino acid sequence comprises a HCDR1 comprising SEQ ID NO: 123, a HCDR2 comprising SEQ ID NO: 124 and a HCDR3 comprising SEQ ID NO: 125; and the VL region amino acid sequence comprises a LCDR1 comprising SEQ ID NO: 126, a LCDR2 comprising SEQ ID NO: 127 and a LCDR3 comprising SEQ ID NO: 128;
(y) the VH region amino acid sequence comprises a HCDR1 comprising SEQ ID NO: 123, a HCDR2 comprising SEQ ID NO: 124 and a HCDR3 comprising SEQ ID NO: 129; and the VL region amino acid sequence comprises a LCDR1 comprising SEQ ID NO: 57, a LCDR2 comprising SEQ ID NO: 58 and a LCDR3 comprising SEQ ID NO: 130;
(z) the VH region amino acid sequence comprises a HCDR1 comprising SEQ ID NO: 131, a HCDR2 comprising SEQ ID NO: 132 and a HCDR3 comprising SEQ ID NO: 133; and the VL region amino acid sequence comprises a LCDR1 comprising SEQ ID NO: 126, a LCDR2 comprising SEQ ID NO: 134 and a LCDR3 comprising SEQ ID NO: 135;
(aa) the VH region amino acid sequence comprises a HCDR1 comprising SEQ ID NO: 131, a HCDR2 comprising SEQ ID NO: 132 and a HCDR3 comprising SEQ ID NO: 136; and the VL region amino acid sequence comprises a LCDR1 comprising SEQ ID NO: 57, a LCDR2 comprising SEQ ID NO: 58 and a LCDR3 comprising SEQ ID NO: 137;
(bb) the VH region amino acid sequence comprises a HCDR1 comprising SEQ ID NO: 54, a HCDR2 comprising SEQ ID NO: 138 and a HCDR3 comprising SEQ ID NO: 139; and the VL region amino acid sequence comprises a LCDR1 comprising SEQ ID NO: 57, a LCDR2 comprising SEQ ID NO: 58 and a LCDR3 comprising SEQ ID NO: 140; or
(cc) the VH region amino acid sequence comprises a HCDR1 comprising SEQ ID NO: 81, a HCDR2 comprising SEQ ID NO: 82 and a HCDR3 comprising SEQ ID NO: 141; and the VL region amino acid sequence comprises a LCDR1 comprising SEQ ID NO: 142, a LCDR2 comprising SEQ ID NO: 143 and a LCDR3 comprising SEQ ID NO: 144.

2. The antibody or antigen-binding portion of claim 1, wherein
(a) the VH region amino acid sequence comprises SEQ ID NO: 1 and the VL region amino acid sequence comprises SEQ ID NO: 2;
(b) the VH region amino acid sequence comprises SEQ ID NO: 3 and the VL region amino acid sequence comprises SEQ ID NO: 2;
(c) the VH region amino acid sequence comprises SEQ ID NO: 4 and the VL region amino acid sequence comprises SEQ ID NO: 2;
(d) the VH region amino acid sequence comprises SEQ ID NO: 5 and the VL region amino acid sequence comprises SEQ ID NO: 6;
(e) the VH region amino acid sequence comprises SEQ ID NO: 7 and the VL region amino acid sequence comprises SEQ ID NO: 8;
(f) the VH region amino acid sequence comprises SEQ ID NO: 9 and the VL region amino acid sequence comprises SEQ ID NO: 10;
(g) the VH region amino acid sequence comprises SEQ ID NO: 11 and the VL region amino acid sequence comprises SEQ ID NO: 12;
(h) the VH region amino acid sequence comprises SEQ ID NO: 13 and the VL region amino acid sequence comprises SEQ ID NO: 14;
(i) the VH region amino acid sequence comprises SEQ ID NO: 15 and the VL region amino acid sequence comprises SEQ ID NO: 16;
(j) the VH region amino acid sequence comprises SEQ ID NO: 17 and the VL region amino acid sequence comprises SEQ ID NO: 18;
(k) the VH region amino acid sequence comprises SEQ ID NO: 19 and the VL region amino acid sequence comprises SEQ ID NO: 20;
(l) the VH region amino acid sequence comprises SEQ ID NO: 21 and the VL region amino acid sequence comprises SEQ ID NO: 22;
(m) the VH region amino acid sequence comprises SEQ ID NO: 23 and the VL region amino acid sequence comprises SEQ ID NO: 22;
(n) the VH region amino acid sequence comprises SEQ ID NO: 24 and the VL region amino acid sequence comprises SEQ ID NO: 22;
(o) the VH region amino acid sequence comprises SEQ ID NO: 25 and the VL region amino acid sequence comprises SEQ ID NO: 22;
(p) the VH region amino acid sequence comprises SEQ ID NO: 26 and the VL region amino acid sequence comprises SEQ ID NO: 27;
(q) the VH region amino acid sequence comprises SEQ ID NO: 28 and the VL region amino acid sequence comprises SEQ ID NO: 29;
(r) the VH region amino acid sequence comprises SEQ ID NO: 30 and the VL region amino acid sequence comprises SEQ ID NO: 31;
(s) the VH region amino acid sequence comprises SEQ ID NO: 32 and the VL region amino acid sequence comprises SEQ ID NO: 33;
(t) the VH region amino acid sequence comprises SEQ ID NO: 34 and the VL region amino acid sequence comprises SEQ ID NO: 35;
(u) the VH region amino acid sequence comprises SEQ ID NO: 36 and the VL region amino acid sequence comprises SEQ ID NO: 37;
(v) the VH region amino acid sequence comprises SEQ ID NO: 38 and the VL region amino acid sequence comprises SEQ ID NO: 39;
(w) the VH region amino acid sequence comprises SEQ ID NO: 40 and the VL region amino acid sequence comprises SEQ ID NO: 41;
(x) the VH region amino acid sequence comprises SEQ ID NO: 42 and the VL region amino acid sequence comprises SEQ ID NO: 43;
(y) the VH region amino acid sequence comprises SEQ ID NO: 44 and the VL region amino acid sequence comprises SEQ ID NO: 45;
(z) the VH region amino acid sequence comprises SEQ ID NO: 46 and the VL region amino acid sequence comprises SEQ ID NO: 47;
(aa) the VH region amino acid sequence comprises SEQ ID NO: 48 and the VL region amino acid sequence comprises SEQ ID NO: 49;
(bb) the VH region amino acid sequence comprises SEQ ID NO: 50 and the VL region amino acid sequence comprises SEQ ID NO: 51; or
(cc) the VH region amino acid sequence comprises SEQ ID NO: 52 and the VL region amino acid sequence comprises SEQ ID NO: 53.

3. The antibody or antigen-binding portion of claim 1, wherein the antibody or antigen-binding portion is human or chimeric.

4. The antibody or antigen-binding portion of claim 1, wherein the antibody or antigen-binding portion comprises an immunoglobulin constant region.

5. The antibody or antigen-binding portion of claim 4, wherein the immunoglobulin constant region is IgG, IgE, IgM, IgD, IgA or IgY.

6. The antibody or antigen-binding portion of claim 5, wherein the immunoglobulin constant region is IgG1, IgG2, IgG3, IgG4, IgA1 or IgA2.

7. The antibody or antigen-binding portion of claim 4, wherein the immunoglobulin constant region is immunologically inert.

8. The antibody or antigen-binding portion of claim 4, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A, L235A and G237A or a wild-type human IgG2 constant region, wherein numbering is according to the EU index as in Kabat.

9. The antibody or antigen-binding portion of claim 4, wherein the immunoglobulin constant region comprises any one of SEQ ID NOs: 145-149.

10. The antibody or antigen-binding portion of claim 1, wherein the antibody or antigen-binding portion is an Fab, an Fab′, an F(ab′)2, an Fv, an scFv, a maxibody, a minibody, a diabody, a triabody, a tetrabody, or a bis-scFv.

11. The antibody or antigen-binding portion of claim 1, wherein the antibody is monoclonal.

12. The antibody or antigen-binding portion of claim 1, wherein the antibody or antigen-binding portion is tetrameric, tetravalent or multispecific.

13. The antibody or antigen-binding portion of claim 1, wherein the antibody or antigen-binding portion is a bispecific antibody or antigen-binding portion that binds specifically to a first antigen and a second antigen, wherein the first antigen is VISTA and the second antigen is not VISTA.

14. An immunoconjugate comprising the antibody or antigen-binding portion of claim 1 linked to a therapeutic agent.

15. The immunoconjugate of claim 14, wherein the therapeutic agent is a cytotoxin, a radioisotope, a chemotherapeutic agent, an immunomodulatory agent, a cytostatic enzyme, a cytolytic enzyme, a therapeutic peptide, a therapeutic nucleic acid, an anti-angiogenic agent, an anti-proliferative agent, or a pro-apoptotic agent.

16. A pharmaceutical composition comprising the antibody or antigen-binding portion of claim 1, and a pharmaceutically acceptable carrier, diluent or excipient.

17. A nucleic acid molecule encoding
(a) the VH region amino acid sequence;
(b) the VL region amino acid sequence; or
(c) both the VH and the VL region amino acid sequences of the antibody or antigen-binding portion of claim 1.

18. An expression vector comprising the nucleic acid molecule of claim 17.

19. A recombinant host cell comprising the nucleic acid molecule of claim 17.

20. A method of producing an anti-VISTA antibody or an antigen-binding portion thereof, the method comprising:
culturing the recombinant host cell comprising the expression vector of claim 17 under conditions whereby the nucleic acid molecule is expressed, thereby producing the antibody or antigen-binding portion; and
isolating the antibody or antigen-binding portion from the host cell or culture.

21. A method for inducing an immune response in a subject, comprising administering to the subject a therapeutically effective amount of the antibody or antigen-binding portion of claim 1.

22. The method of claim 21, wherein the immune response is an immune response against cancer cells.

23. The method of claim 21, wherein the immune response is antibody-dependent cellular cytotoxicity, complement-dependent cytotoxicity or antibody-dependent cellular phagocytosis.

24. A method for treating or preventing a cancer in a subject, comprising administering to the subject a therapeutically effective amount of the antibody or antigen-binding portion of claim 1.

25. The method of claim 24, wherein the cancer is a hematologic malignancy or an epithelial cancer.

26. The method of claim 24, wherein the cancer is squamous cell carcinoma of head and neck, melanoma, non-small cell lung cancer or colon cancer.

27. The method of claim 24, wherein the cancer is acute myeloid leukemia (AML).

28. The method of claim 21, wherein the subject overexpresses VISTA.

29. The method of claim 21, wherein the subject overexpresses VISTA on immune cells.

30. The method of claim 29, wherein the immune cells are tumor-infiltrating immune cells.

31. The method of claim 29, wherein the immune cells are myeloid cells or monocytes.

32. The method of claim 21, further comprising administering to the subject an inhibitor of PD-1 or an inhibitor of PD-L1.

33. The method of claim 32, wherein the inhibitor of PD-1 is an anti-PD-1 antibody or antigen-binding portion thereof.

34. The method of claim 32, wherein the inhibitor of PD-L1 is an anti-PD-L1 antibody or antigen-binding portion thereof.

35. A method for treating a tumor in a subject, the method comprising:
(a) obtaining a VISTA:PSGL-1 proximity score for a tumor sample from the subject; wherein the VISTA:PSGL-1 proximity score is been assigned to the tumor sample by a process which comprises:
(i) obtaining an image of tissue that has been removed from the tumor sample and immunohistochemically stained for VISTA and PSGL-1 expression in a manner that allows stained VISTA cells to be distinguished from stained PSGL-1 cells;
(ii) defining in the image one or more regions of interest (ROIs) that comprises neoplastic cells and associated stroma, wherein substantially all of the neoplastic cells and associated stroma in the image are contained in the defined ROIs;
(iii) randomly creating across each defined ROI a plurality of subregions of substantially the same shape and size, wherein each of the subregions defines an area that is large enough to include a spatially proximal pair of a stained VISTA cell and a stained PSGL-1 cell and small enough to exclude pairs of stained VISTA and PSGL-1 cells that are not spatially proximal; and
(iv) calculating the percent of all of the subregions that are positive for both stained VISTA cells and stained PSGL-1 cells to generate the VISTA:PSGL-1 proximity score for the tumor sample,
(b) comparing the VISTA:PSGL-1 proximity score for the tumor sample with a threshold VISTA:PSGL-1 proximity score,
(c) classifying the tumor as biomarker positive or biomarker negative, wherein if the proximity score for the tumor sample is equal to or greater than the threshold proximity score, then the tumor is classified as positive for VISTA:PSGL-1 proximity biomarker, and if the obtained score is less than the threshold score, then the tumor is classified as negative for the VISTA:PSGL-1 proximity biomarker, and
(d) administering to the subject a therapeutically effective amount of the antibody or antigen-binding portion of claim 1 if the tumor is positive for the VISTA:PSGL-1 proximity biomarker.


SEQUENCE LISTING (165 sequences)

SEQ ID NO: 1 - protein, 122 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAREAGGVVGRVDFQHWGQGTLVTVSS

SEQ ID NO: 2 - protein, 108 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQADFLPPWTFGGGTKVEIK

SEQ ID NO: 3 - protein, 122 aa, Homo sapiens
EVQLVESGGGLVQPGGSLRLSCAASGFTFSDYSMNWVRQAPGKGLEWVSYIASDASTIYYADSVKGRFTISRDNAKNSLYLQMNSLRAEDTAVYYCAREAGGVVGRVDFQHWGQGTLVTVSS

SEQ ID NO: 4 - protein, 122 aa, Homo sapiens
EVQLVESGGGLVQPGGSLRLSCAASGFTFSSYPMNWVRQAPGKGLEWVSYISSDAYTIYYADSVKGRFTISRDNAKNSLYLQMNSLRAEDTAVYYCAREAGGVVGRVDFQHWGQGTLVTVSS

SEQ ID NO: 5 - protein, 122 aa, Homo sapiens
EVQLVESGGGLVQPGGSLRLSCAASGFTFDDYSMNWVRQAPGKGLEWVSYISSDADTIYYADSVKGRFTISRDNAKNSLYLQMNSLRAEDTAVYYCAREAGGVLGRVDFQHWGQGTLVTVSS

SEQ ID NO: 6 - protein, 108 aa, Homo sapiens
DIQLTQSPSSLSASVGDRVTITCQASQDISNYLNWYQQKPGKAPKLLIYDASNLGTGVPSRFSGSGSGTDFTFTISSLQPEDIATYYCQQADFVPPWTFGGGTKVEIK

SEQ ID NO: 7 - protein, 122 aa, Homo sapiens
EVQLVESGGGLVQPGGSLRLSCAASGFTFDSYPMNWVRQAPGKGLEWVSYISSDSSTIYYADSVKGRFTISRDNAKNSLYLQMNSLRAEDTAVYYCAREAGGVVGRVDFQHWGQGTLVTVSS

SEQ ID NO: 8 - protein, 108 aa, Homo sapiens
DIQLTQSPSSLSASVGDRVTITCQASQDISNYLNWYQQKPGKAPKLLIYDASNLGTGVPSRFSGSGSGTDFTFTISSLQPEDIATYYCQQADFIPPWTFGGGTKVEIK

SEQ ID NO: 9 - protein, 122 aa, Homo sapiens
EVQLVESGGGLVQPGGSLRLSCAASGFTFSDYSMNWVRQAPGKGLEWVSYIASDASTIYYADSVKGRFTISRDNAKNSLYLQMNSLRAEDTAVYYCAREAGGVSGRVDFQHWGQGTLVTVSS

SEQ ID NO: 10 - protein, 108 aa, Homo sapiens
DIQLTQSPSSLSASVGDRVTITCQASQDISNYLNWYQQKPGKAPKLLIYDASNLETGVPSRFSGSGSGTDFTFTISSLQPEDIATYYCQQADFVPPWTFGGGTKVEIK

SEQ ID NO: 11 - protein, 122 aa, Homo sapiens
EVQLVESGGGLVQPGGSLRLSCAASGFTFKDYSMNWVRQAPGKGLEWVSYISSDADTIYYADSVKGRFTISRDNAKNSLYLQMNSLRAEDTAVYYCAREAGGVKGRVDFQHWGQGTLVTVSS

SEQ ID NO: 12 - protein, 108 aa, Homo sapiens
DIQLTQSPSSLSASVGDRVTITCQASQDISNYLNWYQQKPGKAPELLIYDASNLETGVPSRFSGSGSGTDFTFTISSLQPEDIATYYCQQADFIPPWTFGGGTKVEIK

SEQ ID NO: 13 - protein, 122 aa, Homo sapiens
EVQLVESGGGLVQPGGSLRLSCAASGFTFDDYSMNWVRQAPGKGLEWVSYISSDASSIYYADSVKGRFTISRDNAKNSLYLQMNSLRAEDTAVYYCAREAGGVLGRVDFQHWGQGTLVTVSS

SEQ ID NO: 14 - protein, 108 aa, Homo sapiens
DIQLTQSPSSLSASVGDRVTITCQASQDISNYLNWYQQKPGKAPKLLIYDASNGETGVPSRFSGSGSGTDFTFTISSLQPEDIATYYCQQADFVPPWTFGGGTKVEIK

SEQ ID NO: 15 - protein, 122 aa, Homo sapiens
EVQLVESGGGLVQPGGSLRLSCAASGFTFDDYSMNWVRQAPGKGLEWVSYISSDADTIYYADSVKGRFTISRDNAKNSLYLQMNSLRAEDTAVYYCAREAGGVNGRVDFQHWGQGTLVTVSS

SEQ ID NO: 16 - protein, 108 aa, Homo sapiens
DIQLTQSPSSLSASVGDRVTITCQASQDISNYLNWYQQKPGKAPKLLIYDASNLEGGVPSRFSGSGSGTDFTFTISSLQPEDIATYYCQQADFVPPWTFGGGTKVEIK

SEQ ID NO: 17 - protein, 122 aa, Homo sapiens
EVQLVESGGGLVQPGGSLRLSCAASGFTFSDYSMNWVRQAPGKGLEWVSYISSDASSIYYADSVKGRFTISRDNAKNSLYLQMNSLRAEDTAVYYCAREAGGVVGRVDFQHWGQGTLVTVSS

SEQ ID NO: 18 - protein, 108 aa, Homo sapiens
DIQLTQSPSSLSASVGDRVTITCQASQDISNYLNWYQQKPGKAPKLLIYDASNLETGVPSRFSGSGSGTDFTFTISRLQPEDIATYYCQQADFVPPWTFGGGTKVEIK

SEQ ID NO: 19 - protein, 122 aa, Homo sapiens
EVQLVESGGGLVQPGGSLRLSCAASGFTFSSYSMNWVRQAPGKGLEWVSYISSDADTIYYADSVKGRFTISRDNAKNSLYLQMNSLRAEDTAVYYCAREAGGVAGRVDFQHWGQGTLVTVSS

SEQ ID NO: 20 - protein, 108 aa, Homo sapiens
DIQLTQSPSSLSASVGDRVTITCQASQDISTYLNWYQQKPGKAPKLLIYDASNLETGVPSRFSGSGSGTDFTFTISSLQPEDIATYYCQQADFVPPWTFGGGTKVEIK

SEQ ID NO: 21 - protein, 124 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDMGDSSSYYDWLFDIWGQGTMVTVSS

SEQ ID NO: 22 - protein, 107 aa, Homo sapiens
EIVMTQSPATLSVSPGERATLSCRASQSVSSSLAWYQQKPGQAPRLLIYGASTRATGIPARFSGSGSGTEFTLTISSLQSEDFAVYYCQQFDNYPPTFGGGTKVEIK

SEQ ID NO: 23 - protein, 124 aa, Homo sapiens
QVQLVQSGAEVKKPGSSVKVSCKASGGTFAQYAISWVRQAPGQGLEWMGGIIPIDATAYYAQKFQGRVTITADESTSTAYMELSSLRSEDTAVYYCARDMGDSSSYYDWLFDIWGQGTMVTVSS

SEQ ID NO: 24 - protein, 124 aa, Homo sapiens
QVQLVQSGAEVKKPGSSVKVSCKASGGTFRQYAISWVRQAPGQGLEWMGGIIPIFGDAYYAQKFQGRVTITADESTSTAYMELSSLRSEDTAVYYCARDMGDSSSYYDWLFDIWGQGTMVTVSS

SEQ ID NO: 25 - protein, 124 aa, Homo sapiens
QVQLVQSGAEVKKPGSSVKVSCKASGGTFHNYAISWVRQAPGQGLEWMGGIIPISGDAYYAQKFQGRVTITADESTSTAYMELSSLRSEDTAVYYCARDMGDSSSYYDWLFDIWGQGTMVTVSS

SEQ ID NO: 26 - protein, 124 aa, Homo sapiens
QVQLVQSGAEVKRPGSSVKVSCKASGGTFSNYAISWVRQAPGQGLEWMGGIIPASGEAYYAQKFQGRVTITADESTSTAYMELSSLRSEDTAVYYCARDEGDSSSYYDWLFDIWGQGTMVTVSS

SEQ ID NO: 27 - protein, 107 aa, Homo sapiens
EIVLTQSPATLSVSPGERATLSCRASQSVSSNLAWYQQKPGQAPGLLIYGASTRATGIPARFSGSGSGTEFTLTISGLQSEDFAVYYCQQQDNYPPTFGGGTKVEIK

SEQ ID NO: 28 - protein, 124 aa, Homo sapiens
QVQLVQSGAEVKKPGSSVKVSCKASGGTFANDAISWVRQAPGQGLEWMGGIIPITGEAYYAQKFQGRVTITADESASTAYMELSSLRSEDTAVYYCARDVGDSSSYYDWLFDIWGQGTMVTVSS

SEQ ID NO: 29 - protein, 107 aa, Homo sapiens
EIVLTQSPATLSVSPGERATLSCRASQSVSSNLAWYQQKPGQAPRLLIYGASTRRTGIPARFSGSGSGTEFTLTISSLQSEDFAVYYCQQFDNSPPTFGGGTKVEIK

SEQ ID NO: 30 - protein, 124 aa, Homo sapiens
QVQLVQSGAEVKKPGSSVKVSCKASGGTFSDTAISWVRQAPGQGLEWMGGIIPIFGPAYYAQKFQGRVTITADESTSTAYMELSSLRSEDTAVYYCARDVGDSSSDYDWLFDIWGQGTMVTVSS

SEQ ID NO: 31 - protein, 107 aa, Homo sapiens
EIVLTQSPATLSVSPGERATLSCRASQSVSSNLAWYQQKPGQAPRLLIYGGSTRATGIPARFSGSGSGTEFTLTISSLQSEDFAVYYCQQLDNYPPTFGGGTKVEIK

SEQ ID NO: 32 - protein, 124 aa, Homo sapiens
QVQLVQSGAEVKKPGSSVKVSCKASGGTFSDYAISWVRQAPGQGLEWMGGIIPASGEAYYAQKFQGRVTITADESTSTAYMELSSLRSEDTAVYYCARDVGDSSSDYDWLFDIWGQGTMVTVSS

SEQ ID NO: 33 - protein, 107 aa, Homo sapiens
EIVLTQSPATLSVSPGERATLSCRASHSVSSNLAWYQQKPGQAPRLLIYGASTRATGIPARFSGSGSGTEFTLTISSLQSEDFAVYYCQQGDNYPPTFGGGTKVEIK

SEQ ID NO: 34 - protein, 124 aa, Homo sapiens
QVQLVQSGAEVKKPGSSVKVSCKASGGTFHSDAISWVRQAPGQGLEWMGGIIPASGEAYYAQKFQGRVTITADESTSTAYMELSSLRSEDTAVYYCARDQRDSSDYYDWLFDIWGQGTMVTVSS

SEQ ID NO: 35 - protein, 107 aa, Homo sapiens
ETVLTQSPATLSVSPGERATLSCRASQSVSSNLAWYQQKPGQAPRLLIYGASTRATGIPARFSGSGSGTEFTLTISSLQSEDFAVYYCQQFDWYPPTFGGGTKVEIK

SEQ ID NO: 36 - protein, 124 aa, Homo sapiens
QVQLVQSGAEVKKPGSSVKVSCKASGGTFAQYAISWVRQAPGQGLEWMGGIIPASGEAYYAQKFQGRVTITADESTSTAYMELSSLRSEDTAVYYCARDVGDSSSWYDWLFDTWGQGTMVTVSS

SEQ ID NO: 37 - protein, 107 aa, Homo sapiens
EIVLTQSPATLSVPPGERATLSCRASQPVSSNLAWYQQKPGQAPRLLIYGASTRATGIPARFSGSGSGTEFTLTISSLQSEDFAVYYCQQYDNYPPTFGGGTKVEIK

SEQ ID NO: 38 - protein, 124 aa, Homo sapiens
QVQLVQSGAEVKKPGSSVKVSCKASGGTFSNYAISWVRQAPGQGLEWMGGIIPIFGDAYYAQKFQGRVTITADESTSTAYMELSSLRSEDTAVYYCARDVGDSSSEYDWLFDIWGQGTMVTVSS

SEQ ID NO: 39 - protein, 107 aa, Homo sapiens
EIVLTQSPATLSVSPGERATLSCRASRSVSSNLAWYQQKPGQAPRLLIYGASTRATGIPARFSDSGSGTEFTLTISSLQSEDFAVYYCQQFDNYPPTFGGGTKVEIK

SEQ ID NO: 40 - protein, 122 aa, Homo sapiens
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARDYGSGSYPGGIDYWGQGTLVTVSS

SEQ ID NO: 41 - protein, 107 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQSDHYPWTFGGGTKVEIK

SEQ ID NO: 42 - protein, 118 aa, Homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARTRYALDFDLWGRGTLVTVSS

SEQ ID NO: 43 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQYSDHPRTFGGGTKVEIK

SEQ ID NO: 44 - protein, 123 aa, Homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARVGHGYGDSLGPFDYWGQGTLVTVSS

SEQ ID NO: 45 - protein, 107 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQVDDYPITFGGGTKVEIK

SEQ ID NO: 46 - protein, 122 aa, Homo sapiens
QVQLVQSGAEVKKPGASVKVSCKASGYTFTSYGISWVRQAPGQGLEWMGWISAYSGDTNYAQKLQGRVTMTTDTSTSTAYMELRSLRSDDTAVYYCARDLGYYSSAALFDIWGQGTMVTVSS

SEQ ID NO: 47 - protein, 108 aa, Homo sapiens
EIVLTQSPATLSLSPGERATLSCRASQSVSSYLAWYQQKPGQAPRLLIYDSSNRATGIPARFSGSGSGTDFTLTISSLEPEDFAVYYCQQHDDWPPLTFGGGTKVEIK

SEQ ID NO: 48 - protein, 121 aa, Homo sapiens
QVQLVQSGAEVKKPGASVKVSCKASGYTFTSYGISWVRQAPGQGLEWMGWISAYSGDTNYAQKLQGRVTMTTDTSTSTAYMELRSLRSDDTAVYYCARDLPISAGPAFDIWGQGTMVTVSS

SEQ ID NO: 49 - protein, 107 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQSDILPFTFGGGTKVEIK

SEQ ID NO: 50 - protein, 121 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAREVYDPHYYGMDVWGQGTTVTVSS

SEQ ID NO: 51 - protein, 106 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYNPLPTFGGGTKVEIK

SEQ ID NO: 52 - protein, 119 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDYDDYYMDVWGKGTTVTVSS

SEQ ID NO: 53 - protein, 106 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYCQQANSHWTFGGGTKVEIK

SEQ ID NO: 54 - protein, 9 aa, Homo sapiens
FTFSSYSMN

SEQ ID NO: 55 - protein, 17 aa, Homo sapiens
YISSSSSTIYYADSVKG

SEQ ID NO: 56 - protein, 15 aa, Homo sapiens
AREAGGVVGRVDFQH

SEQ ID NO: 57 - protein, 11 aa, Homo sapiens
QASQDISNYLN

SEQ ID NO: 58 - protein, 7 aa, Homo sapiens
DASNLET

SEQ ID NO: 59 - protein, 10 aa, Homo sapiens
QQADFLPPWT

SEQ ID NO: 60 - protein, 9 aa, Homo sapiens
FTFSDYSMN

SEQ ID NO: 61 - protein, 17 aa, Homo sapiens
YIASDASTIYYADSVKG

SEQ ID NO: 62 - protein, 9 aa, Homo sapiens
FTFSSYPMN

SEQ ID NO: 63 - protein, 17 aa, Homo sapiens
YISSDAYTIYYADSVKG

SEQ ID NO: 64 - protein, 9 aa, Homo sapiens
FTFDDYSMN

SEQ ID NO: 65 - protein, 17 aa, Homo sapiens
YISSDADTIYYADSVKG

SEQ ID NO: 66 - protein, 15 aa, Homo sapiens
AREAGGVLGRVDFQH

SEQ ID NO: 67 - protein, 7 aa, Homo sapiens
DASNLGT

SEQ ID NO: 68 - protein, 10 aa, Homo sapiens
QQADFVPPWT

SEQ ID NO: 69 - protein, 9 aa, Homo sapiens
FTFDSYPMN

SEQ ID NO: 70 - protein, 17 aa, Homo sapiens
YISSDSSTIYYADSVKG

SEQ ID NO: 71 - protein, 10 aa, Homo sapiens
QQADFIPPWT

SEQ ID NO: 72 - protein, 15 aa, Homo sapiens
AREAGGVSGRVDFQH

SEQ ID NO: 73 - protein, 9 aa, Homo sapiens
FTFKDYSMN

SEQ ID NO: 74 - protein, 15 aa, Homo sapiens
AREAGGVKGRVDFQH

SEQ ID NO: 75 - protein, 17 aa, Homo sapiens
YISSDASSIYYADSVKG

SEQ ID NO: 76 - protein, 7 aa, Homo sapiens
DASNGET

SEQ ID NO: 77 - protein, 15 aa, Homo sapiens
AREAGGVNGRVDFQH

SEQ ID NO: 78 - protein, 7 aa, Homo sapiens
DASNLEG

SEQ ID NO: 79 - protein, 15 aa, Homo sapiens
AREAGGVAGRVDFQH

SEQ ID NO: 80 - protein, 11 aa, Homo sapiens
QASQDISTYLN

SEQ ID NO: 81 - protein, 9 aa, Homo sapiens
GTFSSYAIS

SEQ ID NO: 82 - protein, 17 aa, Homo sapiens
GIIPIFGTANYAQKFQG

SEQ ID NO: 83 - protein, 17 aa, Homo sapiens
ARDMGDSSSYYDWLFDI

SEQ ID NO: 84 - protein, 11 aa, Homo sapiens
RASQSVSSSLA

SEQ ID NO: 85 - protein, 7 aa, Homo sapiens
GASTRAT

SEQ ID NO: 86 - protein, 9 aa, Homo sapiens
QQFDNYPPT

SEQ ID NO: 87 - protein, 9 aa, Homo sapiens
GTFAQYAIS

SEQ ID NO: 88 - protein, 17 aa, Homo sapiens
GIIPIDATAYYAQKFQG

SEQ ID NO: 89 - protein, 9 aa, Homo sapiens
GTFRQYAIS

SEQ ID NO: 90 - protein, 17 aa, Homo sapiens
GIIPIFGDAYYAQKFQG

SEQ ID NO: 91 - protein, 9 aa, Homo sapiens
GTFHNYAIS

SEQ ID NO: 92 - protein, 17 aa, Homo sapiens
GIIPISGDAYYAQKFQG

SEQ ID NO: 93 - protein, 9 aa, Homo sapiens
GTFSNYAIS

SEQ ID NO: 94 - protein, 17 aa, Homo sapiens
GIIPASGEAYYAQKFQG

SEQ ID NO: 95 - protein, 17 aa, Homo sapiens
ARDEGDSSSYYDWLFDI

SEQ ID NO: 96 - protein, 11 aa, Homo sapiens
RASQSVSSNLA

SEQ ID NO: 97 - protein, 9 aa, Homo sapiens
QQQDNYPPT

SEQ ID NO: 98 - protein, 9 aa, Homo sapiens
GTFANDAIS

SEQ ID NO: 99 - protein, 17 aa, Homo sapiens
GIIPITGEAYYAQKFQG

SEQ ID NO: 100 - protein, 17 aa, Homo sapiens
ARDVGDSSSYYDWLFDI

SEQ ID NO: 101 - protein, 7 aa, Homo sapiens
GASTRRT

SEQ ID NO: 102 - protein, 9 aa, Homo sapiens
QQFDNSPPT

SEQ ID NO: 103 - protein, 9 aa, Homo sapiens
GTFSDTAIS

SEQ ID NO: 104 - protein, 17 aa, Homo sapiens
GIIPIFGPAYYAQKFQG

SEQ ID NO: 105 - protein, 17 aa, Homo sapiens
ARDVGDSSSDYDWLFDI

SEQ ID NO: 106 - protein, 7 aa, Homo sapiens
GGSTRAT

SEQ ID NO: 107 - protein, 9 aa, Homo sapiens
QQLDNYPPT

SEQ ID NO: 108 - protein, 9 aa, Homo sapiens
GTFSDYAIS

SEQ ID NO: 109 - protein, 11 aa, Homo sapiens
RASHSVSSNLA

SEQ ID NO: 110 - protein, 9 aa, Homo sapiens
QQGDNYPPT

SEQ ID NO: 111 - protein, 9 aa, Homo sapiens
GTFHSDAIS

SEQ ID NO: 112 - protein, 17 aa, Homo sapiens
ARDQRDSSDYYDWLFDI

SEQ ID NO: 113 - protein, 9 aa, Homo sapiens
QQFDWYPPT

SEQ ID NO: 114 - protein, 17 aa, Homo sapiens
ARDVGDSSSWYDWLFDT

SEQ ID NO: 115 - protein, 11 aa, Homo sapiens
RASQPVSSNLA

SEQ ID NO: 116 - protein, 9 aa, Homo sapiens
QQYDNYPPT

SEQ ID NO: 117 - protein, 17 aa, Homo sapiens
ARDVGDSSSEYDWLFDI

SEQ ID NO: 118 - protein, 11 aa, Homo sapiens
RASRSVSSNLA

SEQ ID NO: 119 - protein, 9 aa, Homo sapiens
FTFSSYGMH

SEQ ID NO: 120 - protein, 17 aa, Homo sapiens
VIWYEGSNKYYADSVKG

SEQ ID NO: 121 - protein, 15 aa, Homo sapiens
ARDYGSGSYPGGIDY

SEQ ID NO: 122 - protein, 9 aa, Homo sapiens
QQSDHYPWT

SEQ ID NO: 123 - protein, 10 aa, Homo sapiens
YSISSGYYWG

SEQ ID NO: 124 - protein, 16 aa, Homo sapiens
SIYHSGSTYYNPSLKS

SEQ ID NO: 125 - protein, 11 aa, Homo sapiens
ARTRYALDFDL

SEQ ID NO: 126 - protein, 11 aa, Homo sapiens
RASQSVSSYLA

SEQ ID NO: 127 - protein, 7 aa, Homo sapiens
DASNRAT

SEQ ID NO: 128 - protein, 9 aa, Homo sapiens
QQYSDHPRT

SEQ ID NO: 129 - protein, 16 aa, Homo sapiens
ARVGHGYGDSLGPFDY

SEQ ID NO: 130 - protein, 9 aa, Homo sapiens
QQVDDYPIT

SEQ ID NO: 131 - protein, 9 aa, Homo sapiens
YTFTSYGIS

SEQ ID NO: 132 - protein, 17 aa, Homo sapiens
WISAYSGDTNYAQKLQG

SEQ ID NO: 133 - protein, 15 aa, Homo sapiens
ARDLGYYSSAALFDI

SEQ ID NO: 134 - protein, 7 aa, Homo sapiens
DSSNRAT

SEQ ID NO: 135 - protein, 10 aa, Homo sapiens
QQHDDWPPLT

SEQ ID NO: 136 - protein, 14 aa, Homo sapiens
ARDLPISAGPAFDI

SEQ ID NO: 137 - protein, 9 aa, Homo sapiens
QQSDILPFT

SEQ ID NO: 138 - protein, 17 aa, Homo sapiens
SISSSSSYIYYADSVKG

SEQ ID NO: 139 - protein, 14 aa, Homo sapiens
AREVYDPHYYGMDV

SEQ ID NO: 140 - protein, 8 aa, Homo sapiens
QQYNPLPT

SEQ ID NO: 141 - protein, 12 aa, Homo sapiens
ARGDYDDYYMDV

SEQ ID NO: 142 - protein, 11 aa, Homo sapiens
RASQSISSWLA

SEQ ID NO: 143 - protein, 7 aa, Homo sapiens
DASSLES

SEQ ID NO: 144 - protein, 8 aa, Homo sapiens
QQANSHWT

SEQ ID NO: 145 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 330 aa, Homo sapiens [sequence omitted]

SEQ ID NO: 148 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 150 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 151 - protein, 307 aa, Macaca fascicularis
MGVPTAPEAGCWRWGSLLFALFLAASLAFKVATLYSLYVCPEGQNVTLTCRFFGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSSCVAYPSSSQESENITAAALATGACIVGILCLPLILLLVYKQRQAASNRRAQELVRMDSNTQGIENPGFEASSPAQGILEAKVRHPLSYVAQRQPSESGRHLLSEPGTPLSPPGPGDVFFPSLDPVPDSPNFEVI

SEQ ID NO: 152 - protein, 384 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENIRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 389 aa, Macaca fascicularis
FKVATLYSLYVCPEGQNVTLTCRVFGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSSCVAYPSSSQESENIRSVECPPCPAPPVAGPSVFLFPPKPKDTLMIDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 381 aa, Mus musculus
FKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSIRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 170 aa, synthetic construct
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENIRSGGSHHHHHH

SEQ ID NO: 156 - protein, 170 aa, synthetic construct
FKVATLYSLYVCPEGQNVTLTCRVFGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSSCVAYPSSSQESENIRSGGSHHHHHH

SEQ ID NO: 157 - protein, 167 aa, synthetic construct
FKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSIRSGGSHHHHHH

SEQ ID NO: 158 - protein, 5 aa, Homo sapiens
YKTWY

SEQ ID NO: 159 - protein, 5 aa, Homo sapiens
SERRP

SEQ ID NO: 160 - protein, 6 aa, Homo sapiens
TFQDLH

SEQ ID NO: 161 - protein, 9 aa, Homo sapiens
IRHHHSEHR

SEQ ID NO: 162 - protein, 9 aa, Homo sapiens
VQTCSERRP

SEQ ID NO: 163 - protein, 8 aa, Homo sapiens
SHDLAQRH

SEQ ID NO: 164 - protein, 5 aa, Homo sapiens
LVVEI

SEQ ID NO: 165 - protein, 6 aa, Homo sapiens
HHSEHR